(19) United States
(12) Patent Application Publication
(10) Pub. No.: US 2009/0117115 A1
(43) Pub. Date: May 7, 2009

(54) Binary epitope antibodies and B cell superantigen immune stimulants

(76) Inventors: Sudhir Paul, Missouri City, TX (US); Yasuhiro Nishiyama, Houston, TX (US); Stephanie Planque, Houston, TX (US)

Correspondence Address: Benjamin Aaron Adler;ADLER & ASSOCIATES, 8011 Candle Lane, Houston, TX, 77071, US

(21) Appl. No.: 11/983,592

(22) Filed: Nov. 9, 2007

Related U.S. Application Data
(60) Provisional application No. 60/857,764, filed on Nov. 9, 2006.

(51) Int. Cl. A61K 39/395; C07K 14/00; G01N 33/53; A61P 31/18

(52) U.S. Cl. 424/139.1; 530/350; 435/7.1

(57) ABSTRACT

Provided herein are dual epitope polypeptides and electrophilic analogs thereof having a superantigenic epitope and at least one other epitope effective to induce the production of antibodies with binary specificity for the epitopes on a polypeptide antigen. Also, provided are electrophilic analogs of polyclonal B cell stimulants or lipids, polysaccharides and lipopolysaccharides or nucleotides. In addition, provided herein are methods for increasing production of binary epitope specific antibodies recognizing B cell polypeptide antigens, for stimulating production of antibodies using one or more of the polypeptides or electrophilic analogs and for isolating binary epitope specific antibodies or fragments thereof. Further provided are the antibodies so produced and methods of treating HIV using the same.

### CROSS-REFERENCE TO RELATED APPLICATIONS

This nonprovisional application claims benefit of priority of provisional U.S. Ser. No. 60/857,764, filed Nov. 9, 2006, now abandoned, and the entirety of which is hereby incorporated.

### FEDERAL FUNDING LEGEND

This invention was produced in part using funds obtained through grants AI058865, AI067020, AI071951 and AI062455 from the National Institutes of Health. Consequently, the federal government has certain rights in this invention.

### BACKGROUND OF THE INVENTION

1. Field of the Invention

The present invention relates to the fields of immunology, virology and medicine. Specifically, the present invention relates to binary epitope reactive antibodies to B cell superantigenic polypeptides and to immune stimulants that induce the production of such antibodies and to methods and reagents permitting the identification and induction of the binary epitope reactive antibodies with the ability to catalyze the hydrolysis of the superantigenic polypeptide or bind the polypeptide with covalent character.

2. Description of the Related Art

Unlike conventional antigens, B cell superantigenic polypeptides are recognized by antibodies (immunoglobulins) present in the preimmune repertoire without the requirement for prior exposure to the antigen. This is because the superantigen recognition property is encoded by inherited variable (V) region genes (germline V genes) that are utilized to synthesize the V domains of antibodies. Conventional antigen recognition by antibodies requires adaptive sequence diversification of the V domains occurring over the course of B cells that are stimulated with antigens. The recognition of conventional antigens occurs mainly at the complementarity determining regions (CDRs) of the V domains. Superantigen recognition by antibodies is thought to occur without the requirement of V domain adaptive diversification, and the recognition process is thought to occur mainly at V domain framework regions (FRs).

Improvement of the antigen recognition capability of antibodies is driven by contact of the antigen with immunoglobulin expressed on the surface of B lymphocytes as a component of the B cell receptor (BCR), which in turn drives cell division and B cell clonal selection. Superantigen contact with the BCR, on the other hand, can drive the B cells into the programmed cell death pathway after a transient proliferative phase. There are no reports of adaptive improvement of superantigen recognition by antibodies. Evidently, the immune system is unable to mount protective adaptive responses to the harmful effects of superantigenic polypeptides. The antibodies present in the preimmune immune repertoire recognize superantigens with moderate to low affinity, and they offer only limited defense against superantigen harmful effects.

Polypeptides that can be classified as B cell superantigens include the HIV proteins gp120 and Tat, the Staphylococcus aureus protein Protein A) and the Streptococcus protein Protein L. The list of harmful B cell superantigenic polypeptides can be expected to grow as additional antigens are studied, and immunological assay sensitivity is improved.

The HIV envelope protein gp120 is a target for experimental vaccines that attempt to induce the synthesis of neutralizing antibodies (Abs). Most Abs induced by immunization with gp120 are directed to the highly mutable regions of the protein, particularly the third variable domain (V3 domain). These Abs neutralize the infecting viral strain. However, the gp120 V domains mutate over the course of infection, resulting in viral escape mutants refractory to neutralization by the Abs. Moreover, the gp120 V domains of various HIV strains responsible for the pandemic in different parts of the world are highly divergent, and Abs induced by immunization with gp120 usually fail to neutralize heterologous HIV strains. This has lead to the search for structurally conserved neutralized epitopes expressed by gp120. Candidate epitopes are the comparatively conserved regions of gp120 involved in HIV binding to host cell receptors, i.e., CD4 and chemokine receptors. Regrettably, these epitopes are poorly immunogenic. Rare monoclonal Abs (MAbs) directed to regions close to the CD4 and chemokine receptor binding sites of gp120 have been identified using complex experimental protocols. These MAbs neutralize many but not all clade B HIV-1 strains.

The CD4 binding site (CD4bs) is thought to be a discontinuous determinant composed of residues 256, 257, 368-370, 421-427 and 457. The CD4bs is suggested to undergo a conformational transition(s) when the gp120 trimer expressed on the viral surface is shed as soluble monomers, and Abs to the monomer CD4bs are often poorly reactive with the trimer CD4bs. Moreover, the 421-427 peptide region of the CD4bs is an important component of the B lymphocyte superantigen site of gp120; gp120_{SAg}; defined as a site to which Abs are present in the preimmune repertoire without the requirement for adaptive sequence diversification of the Ab variable domains). As in the case of other B cell superantigens, gp120_{SAg} binding to Ig expressed as part of the B cell receptors (BCRs) is thought to induce B cell apoptosis, and there is no evidence for adaptive amplification of gp120SAg binding Abs in HIV infected subjects. To the contrary, HIV infected subjects express diminished serum levels of VH3+ Igs, the VH family thought to bind gp120_{SAg} preferentially.

No vaccine or immune therapy for HIV infection is presently available. Thus, a recognized need is still present in the art for immune prophylactics and therapeutics to HIV. Specifically, the prior art is deficient in antibodies with binary epitope specificity having increased virus neutralizing activity and methods of producing the same. The present invention fulfills this long standing need in the art.

### SUMMARY OF THE INVENTION

The present invention describes a hitherto unsuspected property of certain antibodies, the ability to recognize two distinct epitopes of gp120. This antibody property is hereafter designated ‘binary epitope specificity’.

The present invention is also directed to a polypeptide or electrophilic analog thereof that comprises a superantigenic epitope and at least one other epitope effective to induce the production of antibodies with binary specificity for the corresponding epitopes on a polypeptide antigen. In a related invention the polypeptide or electrophilic analog thereof further comprises one or more electrophilic groups therewithin, where the electrophilic groups effective to induce synthesis of binary specific antibodies with enhanced nucleophilic reactivity. Conventional antibodies bind antigens reversibly by noncovalent mechanisms. Like conventional antibodies, chemically reactive antibodies initially recognize the antigen by noncovalent means. However, nucleophilic sites located in the V domains of chemically reactive antibodies then proceed to recognize electrophilic reaction centers in the antigen. This has two consequences, formation of irreversible immune complexes with covalent nucleophile-electrophile pairing, and, if a water molecule is available at the reaction center, the hydrolysis of the antigen by the antibody. The irreversible binding property enhances antibody potency, because it precludes regeneration of active antigen by dissociation of the immune complexes. Similarly, the catalytic hydrolysis step results in irreversible antigen inactivation, as the antigen fragments do not usually express biological activity similar to the parent antigen.

The present invention also is directed to a method for producing binary epitope specific antibodies to a B cell polypeptide superantigen. The method comprises administering one or both of the polypeptide construct or an electrophilic polypeptide analog thereof described herein to a living animal. Inclusion of two epitopes in the polypeptide construct can stimulate the production by B cells of antibodies that recognize one of the epitopes by interactions at antibody complementarity determining regions (CDRs), and of the second epitope, by interactions at the framework regions (FRs). Recognition of the former epitope is driven by conventional B cell clonal selection processes. The cellular proliferative signal generated by interaction at the CDRs is sufficient to overcome the negative effect of interactions of the second, superantigenic epitope at the FRs. This results in production of antibodies that express binary epitope specificity.

In a related invention the method comprises a further step of administering one or more immunological adjuvants effective to stimulate T-cell independent or T-cell dependent B cell antibody production to the living animal.

The present invention is directed further to a method for production of antibodies recognizing the B cell superantigenic site of HIV gp120. The method comprises administering to a living animal a combination of a dual epitope polypeptide and electrophilic analog thereof. In a related invention the method comprises a further step of administering one or more immunological adjuvants effective to stimulate T-cell independent or T-cell dependent B cell antibody production to the living animal.

The present invention is directed further still to a method for stimulating increased production of antibodies to superantigens found in the preimmune immune repertoire of living organisms. The method comprises administering one or both of a polyclonal B cell stimulant or an electrophilic analog thereof to an living animal.

The present invention is directed further still to the antibodies produced by the methods described supra. A related invention is directed to methods of treating HIV infection in a subject. The methods comprise administering an immunologically effective amount of the antibodies described herein to the subject.

The present invention is directed further still to a method for isolating an individual antibody or antibody fragment thereof having a unique sequence and binary epitope specificity from an antibody repertoire. The method comprises displaying the antibody repertoire on the surface of phage particles and screening the antibody repertoire with a polypeptide or a polypeptide electrophilic analog thereof or a stimulant or stimulant electrophilic analog thereof, wherein an antibody or antibody fragment thereof reacting with the polypeptide, the stimulant or the electrophilic analogs thereof thereby isolates the binary epitope specific antibody or antibody fragment thereof from the antibody repertoire.

The present invention is directed further still to electrophilic analogs of polypeptides or lipids, polysaccharides and lipopolysaccharides or oligonucleotides having the general structure

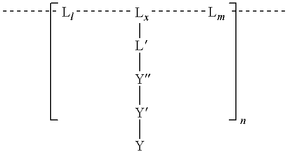

In a polypeptide electrophilic analog L₁ . . . Lx . . . Lm are components defining an antigenic determinant where Lx is a component amino acid of the antigenic determinant, L′ is a functional group of Lx, Y″ is a molecule, a covalent bond or a linker, Y′ an optional charged or neutral group, Y is an electrophilic group that reacts covalently with an antibody that binds to said antigenic determinant, n is an integer from 1 to 1000, and m is an integer from 1 to 30. In a lipid, polysaccharide and lipopolysaccharide electrophilic analog L₁ . . . Lx . . . Lm are components defining a receptor binding determinant where Lx is a component sugar or lipid of the receptor binding determinant, L′ is a functional group of Lx, Y″ is a molecule, a covalent bond or a linker, Y′ an optional charged or neutral group, Y is an electrophilic group that reacts covalently with a cellular receptor that binds to said receptor binding determinant, or an acyl group; wherein, optionally, Y″, Y′ or Y comprises a water-binding group as a terminal or internal component, n is an integer from 1 to 1000, and m is an integer from 1 to 1000. In a nucleotide electrophilic analog L₁ . . . Lx . . . Lm are nucleotide components defining a receptor binding determinant where Lx is a component sugar or lipid of the receptor binding determinant, Lx is a component nucleotide of the receptor binding determinant, L′ is a functional group of Lx, Y″ is a molecule, a covalent bond or a linker, Y′ an optional charged or neutral group, Y is an electrophilic group that reacts covalently with a cellular receptor that binds to said receptor binding determinant, or an acyl group; wherein, optionally, Y″, Y′ or Y comprises a water-binding group as a terminal or internal component, n is an integer from 1 to 1000, and m is an integer from 1 to 1000.

Other and further aspects, features and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 depicts a dual epitope recognition model. Shown is a schematic representation of proposed IgG recognition of the V3 stem (301-311) and SAg peptide region (421-433) by, respectively, the adaptively matured CDRs and conserved FRs. Nu, nucleophilic residue.

FIGS. 2A-2B show E-gp120 immunogens. FIG. 2A depicts schematic structures of E-gp120. Electrophilic phosphonate groups were placed on Lys side chains of gp120 using two alternate linkers. Biotin (˜0.7/gp120 mol) was introduced into gp120 prior to phosphonate introduction. FIG. 2B shows streptavidin-peroxidase stained blots of SDS-gels showing E-gp120 oligomers. Lanes 1-3 show E-gp120 preparations (incubated for 4 h in a neutral pH buffer; biotinylated) with phosphonate/gp120 ratios of 4.4, 14.2 and 23.4, respectively.

FIG. 3 demonstrates HIV-1 neutralization by MAbs YZ18 and YZ23. IgG CRL1689 is an isotype matched IgG. HIV strain ZA009, clade C, R5-dependent. Peripheral blood mononuclear cell hosts. Neutralization was computed as % decrease of p24 concentrations in MAb-containing wells compared to vehicle control.

FIG. 4 demonstrates inhibition of gp120-MAb binding by gp120 fragment peptides. ELISA assays were conducted using MAbs (5 μg/mL) in gp120 (YZ23) or E-gp120 1a (YZ18) coated plates (40 ng/well) in the presence or absence of the indicated competitor peptides. Bound MAbs were measured using anti-mouse IgG-peroxidase. Peptides 301-315 and 417-431 inhibited the binding with potency equivalent to 297-311 and 421-433, respectively (not shown). Also shown are ELISA results using MAb SK-T03 (0.2 μg/mL) and immobilized gp120 in the presence or absence of peptide 465-479. A non-inhibitory control peptide is shown in each panel.

FIGS. 5A-5D demonstrate dual epitope binding by anti-E-gp120 MAbs. FIG. 5A shows structures of electrophilic probes used for dual binding studies. Shown are E-293-311, E421-433 and electrophilic hapten probes. Control is E-VIP. FIG. 5B demonstrates E-421-433 and E-293-311 binding by IgGs YZ18. Streptavidin-peroxidase stained blot of reducing SDS-gels showing complexes formed by incubating (6 h) IgG YZ18 (0.5 μM) with E-peptide probes or E-hapten (10 μM). FIG. 5C demonstrates recognition of 421-433-CRA and 293-311-CRA by IgGs YZ23. ELISA data using IgG YZ23 and biotinylated peptide probes (immobilized on streptavidin plates; 0.4 μg/well). After incubation with IgG, the wells were treated with PBS (total binding) or buffer containing 2% SDS (SDS-resistant binding). An isotype-matched irrelevant IgG (clone CRL1689) did not show detectable binding (27 μg/mL, 0.04±0.01). FIG. 5D depicts the ternary complexation of IgG YZ23 with E-293-311 and E-421-433. Shown in the left panel is the time course of E-293-311 binding by IgG YZ23. IgG YZ23, 1 μM; E-293-311, 10 μM. The reaction mixtures were subjected to SDS-electrophoresis and biotin content in the E-293-311/IgG complexes was determined by densitometry of streptavidin-peroxidase stained blots. Shown in the right panel is E-421-433 binding by IgG YZ23-E-293-311 complexes. IgG YZ23 was allowed to react with E-293-311 to near saturation (3 h), then with E-421-311 (10 μM) for 8 h. Biotin content in the complexes was determined as in the left panel.

FIGS. 6A-6D demonstrate the antibody structural basis for dual epitope recognition. FIG. 6A shows residues 301-311 (yellow) and 421-433 (green) in the crystal structure of gp120 (PDB 2B4C). The two peptide regions are distant from each other, and exposed on the different surface of gp120. FIG. 6B shows the alignment of VH sequences of MAbs YZ18 and YZ23 with 4 VH3 family Abs that recognize gp120 as a superantigen. Underlined are CDRs. Top number, linear numbering of 18/2; Botton number, linear numbering of YZ18. Bold, putative VH residues involved in gp120 superantigen interactions (Karray et al, JI 1998, 161, 6681). YZ18 and YZ23 residues highlighted in red represent identities to gp120SAg binding residues in a VH3 Ab; Highlighted in cyan, conserved changes (following the amino acid homology definition by Karray et al.). FIG. 6C shows the putative dual binding cavities in MAb YZ23. X-ray diffraction data for the YZ23 Fab crystal were collected from synchrotron radiation source and its structure was solved by molecular replacement method at 2.5 Å resolution. The putative 301-311 antigen binding cavity (Ag cavity; green) is formed by Ab CDRs. The 421-433 binding cavity (SAg cavity; white) is formed by the framework residues reported to be responsible for gp120 SAg binding. FIG. 6D depicts a dual epitope recognition model. The V3 stem epitope (301-311) and SAg epitope (421-433) are recognized by, respectively, the adaptively matured CDRs (Ag cavity) and conserved FRs (SAg cavity). Nu, nucleophilic residue. The model is feasible if two Ab binding subsites are simultaneously in register with the two gp120 regions. Flexibility of the gp120 region linking the V3 stem and the SAg peptide region may be important in this model. The V3 loop, particularly the crown is highly flexible. The Ab nucleophile-gp120 electrophile pairing results in either irreversible complexation or proteolytic cleavage.

FIGS. 7A-7C depict the immunogen structural basis for dual epitope recognition. FIG. 7A shows the covalent oligomerization of E-gp120. Presented in the left panel are silver-stained SDS electrophoresis gels showing the presence of covalent oligomers in E-gp120 (lane 1), the trimer enriched fraction from gel filtration chromatography on Superose 6 (lane 2), and the control gp120 devoid of phosphonate groups (lane 3). Shown in the right panel is gel filtration chromatogram, in which the hatched area represents the trimer enriched fraction used for lane 2 in the left panel. FIG. 7B demonstrates the inhibitory effect of E-gp120 in HIV binding by MAbs directed to CD4 binding site (b12), V3 apex (447-52D) and conformational carbohydrate (2G12) epitopes. MAbs (b12, 45 μg/mL; 447-52D, 0.8 μg/mL; 2G12, 7.5 μg/mL) were incubated with HIV (MN, 1.6×10³ TCID₅₀/mL) in the presence or absence of gp120, E-gp120 1a (32 phosphonate groups/gp120), EGF, or E-EGF (0.5 μM) for 20 h. MAb-bound virions were captured in protein G-coated wells (1 μg/well; 1 h), lysed with 10% Triton X100, and HIV p24 in the lysates were measured by ELISA. FIG. 7C shows E-gp120 binding by MAbs directed to CD4 binding site (b12), V3 apex (447-52D) and conformational carbohydrate (2G12) epitopes. ELISA plates were coated with E-gp120 1a, trimer enriched preparation of 1a (purified by gel filtration), or control gp120 (40 ng/well) and bound MAbs detected by anti-human IgG-HRP.

FIG. 8 demonstrates the neutralizing activity of dual binding and non-dual binding MAbs. The anti-E-gp120 MAb panel (n=17) was assessed for dual epitope binding and HIV neutralization activities. The dual binding activity was examined by the electrophoresis assay using E-293-311 and E-421-433 as in FIG. 5B (IgG, 0.5 μM; E-peptides, 10 μM; 3 h). MAbs positive for E-293-311 binding and E-421-433 binding were defined as those yielding band intensities>18220 and >9110 AVU (arbitrary volume unit), respectively (mean band intensity of control probe adducts of 17 MAbs, 1822 AVU). HIV neutralization was studied using a clade C primary isolate ZA009 and PBMCs as host cells. MAbs that displayed >50% neutralization at <30 μg/mL were considered positive.

FIGS. 9A-9C demonstrates irreversible gp120 binding by anti-E-gp120 MAbs. FIG. 9A depicts an ELISA showing SDS-resistant gp120 binding by anti-E-gp120 MAbs (YZ series, 75 μg/ml; SKT03, 1 μg/ml). Three control anti-V3 MAbs (IgG #1121, 75 μg/ml, Immunodiagnostics Inc; 257-D IV, 1 μg/ml; 268-D IV, 15 μg/ml) and a control anti-CD4bs MAb b12 (10 μg/ml) are also shown. Values (means of 3 replicates) represent residual SDS-resistant binding (plates washed with 2% SDS following MAb binding to immobilized gp120) expressed as % of total binding (plates washed with PBS, pH7.4, following MAb binding to gp120). 40 ng gp120/well. FIG. 9B demonstrates SDS-resistant gp120-binding by MAb SK-T03. Shown are streptavidin-peroxidase stained (lanes 1-3) and anti-mouse IgG-peroxidase stained (lanes 4-6) blots of non-reducing SDS-electrophoresis gels showing SDS-resistant adducts formed by treatment of gp120 with MAb SK-T03. Lanes 1 and 4, MAb SK-T03 incubated with Bt-gp120; lane 2, Isotype-matched control MAb MOPC21 incubated with Bt-gp120; lanes 3 and 5, Control Bt-gp120 alone incubated in the diluent; lane 6, MAb SK-T03 alone incubated in the diluent. Ab, 75 (lanes 1-3) or 15 μg/mL (lanes 4-6); Bt-gp120, 5 (lanes 1-3) or 27 μg/mL (lanes 4-6); Incubations for 17 h. Nominal molecular weights computed by comparison with standard proteins are indicated. FIG. 9C demonstrates the stability of MAb SK-T03 immune complexes in non-denaturing solution. Immune complexes were formed by incubating MAb SK-T03 (20 μg/mL) or MAb 268-D IV (1 μg/mL) with Bt-gp120 (0.2 μg/mL) for 12 h. The complexes were captured on protein G-Sepharose, free gp120 removed by washing and the resin was incubated further in the presence of gp120 peptide 465-479 (MAb SK-T03; 10 μg/mL) or peptide 309-323 (MAb 268-D IV; 10 μg/mL). Aliquots were withdrawn at 0, 4, 30, 73, 121 and 239 h, and the residual immune complexes were detected using a streptavidin-peroxidase conjugate. Values are expressed relative to the binding at the t=0 (A490 for MAb SKT-T03 and MAb 268-D IV, respectively, 1.05±0.08 and 1.78±0.07) and are corrected for background binding observed using an irrelevant MAb (MOPC 21; 20 μg/mL), which was nearly constant at all of the time point studied (A490, 0.23±0.01).

FIGS. 10A-10B depict cleavage of gp120 by IgG YZ18. FIG. 10A is a streptavidin-peroxidase stained blot of SDS-gels showing time dependent cleavage of biotinylated gp120 by IgG YZ18 and lack of cleavage by IgG YZ19. IgG, 1 μM; Bt-gp120, 0.2 μM; 22 h incubation. OE, Overexposed lanes showing Bt-gp120 incubated for 22 h in diluent or IgG YZ18 IgG (1 μM). Product bands at 27 kDa and 15 kDa are visible in addition to the major 50-55 kDa bands. FIG. 10B is an anti-gp120-peroxidase stained blot of SDS-gel showing gp120 incubated with diluent or IgG YZ18 IgG. IgG, 1 μM; gp120, 1 μM; 24 h incubation.

FIG. 11 depicts a proposed reaction mechanism of nucleophilic Abs: Antigen hydrolysis (top) and irreversible binding (bottom). The Ab forms the initial noncovalent complex by conventional epitope-paratope interactions. In proteolysis, the active site nucleophile attacks the carbonyl of the scissile bond in the antigen to form the tetrahedral transition-state complex. The C-terminal antigen fragment is released and the acyl-Ab complex is formed. Hydrolysis of the acyl-Ab complex (deacylation) releases the N-terminal antigen fragment and regenerates the catalytic Ab. In irreversible binding, accumulation of the tetrahedral complex (IC′) or the trigonal acyl-Ab complex (IC″, after release of the C-terminal antigen fragment) may occur because of increased stability of the bond formed by the attacking nucleophile or deficient support for a step(s) after completion of the nucleophile attack. NuH, nucleophile; Ag₁—NH—CH(R)—CO₂H, N-terminal antigen fragment; NH₂—Ag₂, C-terminal antigen fragment.

FIG. 12 shows a hypothetical representation of immune response to the dual epitope construct.

FIGS. 13A-13D show dual epitope constructs. FIG. 13A is (301-311)-GMB-GGS-(E421-433); GMB, γ-maleimidobutyryl. FIG. 13B is T-(301-311)-GMB-GGS-(E-c421-433); 0, ornithine. Shown is a constrained E-421-433 variant as a component of the dual-epitope construct. The T-epitope (7) is shown within the rectangle. FIG. 13C is KLH-(301-311)-GMB-GGS-(E-c421-433). Shown is a KLH-conjugated dual epitope construct containing 301-311 and constrained E-421-433 (Ec421-433) connected by a GMB-GGS linker. FIG. 13D is E-HIV. Psoralen-inactivated HIV particles with phosphonates placed on Lys residues. FIG. 13E depicts a whole virus particle showing coat proteins modified to display electrophilic phopshonate groups.

FIG. 14 is a schematic diagram of natural and engineered antibody versions. Monomer IgA and IgG are distinguished by their (black) and (grey) constant domains. Secretory IgA is composed of dimeric and polymeric IgA stabilized by the J chain (which binds the tail piece) and the secretory component. IgM is a disulfide-linked pentamer containing the constant domain. Antigen binding and catalytic cleavage occurs at the variable regions of the light and heavy chains (red and blue, respectively). The variable domains linked by a peptide linker is referred to as a single chain Fv.

FIGS. 15A-15C demonstrate cleavage of biotinylated gp120 by serum and salivary IgA from humans without HIV infection. FIG. 15A shows streptavidin-peroxidase stained blots of reducing SDS-gels showing time-dependent cleavage of Bt-gp120 (0.1 μM) incubated with pooled polyclonal serum IgA (160 μg/ml) and salivary IgA (32 μg/ml) from 4 humans. Reaction volume, 0.02 ml. Diluent lane, gp120 incubated with diluent instead of IgA. OE, overexposed lane showing Bt-gp120 incubated for 46 h with salivary IgA. FIG. 15B shows the comparative gp120 cleaving activity of salivary IgA (32 μg/ml) and serum IgA (144 μg/ml) from 4 humans expressed per equivalent Ab mass. Reaction conditions: 17 h, 0.1 μM Bt-gp120. FIG. 15C shows the comparative gp120 cleaving activity of serum IgA and IgG (144 μg/ml) expressed per equivalent Ab mass. IVIG, commercial IgG preparations (the 3 data points correspond to the following IVIG preparations: Intratect, Gammagard, Inveegam). Each IgA and IgG point represents Abs from a different human.

FIGS. 16A-16C depict EP-hapten 1 interactions with IgA. FIG. 16A shows the EP-hapten 1 structure. The control non-electrophilic phosphonic acid hapten 2 is structurally identical to hapten 1 except for the absent phenyl groups. FIG. 16B demonstrates inhibition of catalysis and irreversible binding by EP-hapten 1. gp120 (0.1 μM) was reacted with salivary IgA (2 μg/ml) or serum IgA (160 μg/ml) in the absence or presence of EP-hapten 1 and control hapten 2 (1 mM) for 8 h before incubation with non-biotinylated gp120 for 16 h. Data are from SDS-electrophoresis gels stained with peroxidase-conjugated polyclonal anti-gp120. % Inhibition=[(100-gp120 cleaved in the presence of inhibitor)/(gp120 cleaved in the absence of inhibitor)]×100]. Values are means of duplicates. FIG. 16C shows streptavidin-peroxidase stained blots of reducing SDS-gels showing EP-hapten 1 and hapten 2 treated salivary IgA and serum IgA. H and L denote heavy and light chain subunit bands, respectively.

FIG. 17 demonstrates preferential cleavage of gp120 by IgA and sIgA. Biotinylated (Bt) proteins studied are gp120, soluble epidermal growth factor receptor (sEGFR), bovine serum albumin (BSA), C2 domain of human coagulation factor VIII (C2), and HIV Tat. Shown are streptavidin-peroxidase stained blots of reducing SDS-gels of the proteins (0.1 μM) incubated (17 h) with serum IgA, salivary IgA (both 160 μg/ml) or diluent.

FIGS. 18A-18C depict inhibition of gp120_{SAg} hydrolysis and irreversible IgA binding by EP-421-433. FIG. 18A demonstrates preferential inhibition of IgA catalyzed gp120 cleavage by EP-421-433. Salivary IgA (16 μg/ml) or serum IgA (160 μg/ml) were preincubated (6 h) with EP-421-433 or EP-VIP (100 μM), the reaction mixtures were incubated further for 16 h following addition of gp120 (0.1 μM). Inhibition of gp120 cleavage determined as in FIG. 3. FIG. 18B demonstrates irreversible binding of EP-421-433 by serum IgA and salivary IgA. Shown are streptavidin-peroxidase stained blots of reducing electrophoresis gels of IgA (80 μg/ml) incubated with EP-421-433, EP-VIP or EP-hapten 1 (10 μM; reaction time, 21 h). H and L denote heavy chain and light chain bands. FIG. 18C demonstrates inhibition of irreversible IgA:EP-421-433 binding by gp120 peptide 421-435. Salivary IgA (80 μg/ml) was treated with gp120 peptide 421-435 (100 μM) or diluent followed by addition of EP-421-433 (10 μM) and incubation for 21 h. EP-421-433 adducts were detected. Plotted are the aggregate intensities of the heavy and light chain subunits determined by densitometry.

FIG. 19 identifies peptide bonds cleaved by salivary IgA. Shown are the Coomassie blue-stained SDS-gel electrophoresis lanes of gp120 (270 μg/ml) incubated for 9 h in diluent (lane 1) or with IgA (80 μg/ml) (lane 2). Lane 3 shows the electrophoretic profile of the gp120-IgA reaction mixture following more prolonged digestion (46 h). The yields of the indicated amino acid were 0.3-1.5 pmol. A mixture of PTH-amino acids (2 pmol each; Applied Biosystems) was employed as standard (sensitivity of detection of individual amino acids, 0.04-0.10 pmol).

FIGS. 20A-20C demonstrate HIV neutralization by Abs from HIV-seronegative humans. FIG. 20A shows neutralizing potency of IgA and IgG Abs purified from pooled serum or saliva of 4 human subjects. IVIG, Gammagard S/D. HIV-1 strain, 97ZA009; host cells, phytohemagglutinin-stimulated PBMCs. Abs were incubated with the virus for 24 h. Values are expressed as percent reduction of p24 concentrations in test cultures compared to cultures that received diluent instead of the Abs (means±s.d. of 4 replicates). FIG. 20B shows inhibition of IgA neutralizing activity (pooled from 34 donors) by EP-421-433. IgA purified from human serum (2 μg/ml) was preincubated (0.5 h) with EP-421-433 (100 μM), control EP-VIP or diluent, and the neutralizing activity was determined as in panel A. FIG. 20C demonstrates time-dependent HIV neutralizing activity. HIV was preincubated with the salivary or serum IgA for 1 h and the neutralizing activity measured as in FIG. 20A.

FIG. 21 shows gp120 cleavage activity of Abs from mice immunized with KLH conjugated E-421-433. Mice were immunized with KLH-E-421-433 intraperitoneally with RIBI adjuvant or intranasally with IL12/CTB adjuvant or LTm adjuvant. Gp120 cleavage activity of purified Abs was determined by the electrophoresis assay using Bt-gp120 (0.1 μM) as a substrate.

FIGS. 22A-22D illustrate binding, catalytic and neutralizing response to intranasal immunization with KLH conjugated E-421-433. FIG. 22A shows the catalytic response. Shown is specific gp120 cleaving activity (nM/h/μg Ig) of Abs purified from serum, saliva, and vaginal wash of preimmune mice and mice immunized nasally with E421-433 KLH conjugate (LTm adjuvant; 4 nasal immunizations, samples obtained 1 wk thereafter). Inset, streptavidin-peroxidase stained blots of SDS-gels showing Bt-gp120 (0.1 μM) incubated in the presence or absence of hyperimmune vaginal IgA (3 μg/mL) for 17 h. FIG. 22B shows the binding response. ELISA values for E-421-433 binding by purified Abs from preimmune and hyperimmune mice (nasal immunization, same samples as in FIG. 22A) detected by peroxidase conjugates of anti-mouse IgG, IgM, or IgA. FIG. 22C shows the IgM neutralizing response. IgMs purified from serum of preimmune and E-421-433-immunized mice (intraperitoneal or nasal immunizations in the indicated adjuvants) were analyzed for HIV neutralizing activity using the clade C primary isolate ZA009 and PBMC hosts. FIG. 22D shows the IgG and IgA neutralizing responses. IgGs and IgAs are purified from serum of preimmune and E-421-433-immunized mice (nasal immunizations with LTm as adjuvant) were analyzed for HIV neutralizing activity using the clade C primary isolate ZA009 and PBMC hosts.

FIGS. 23A-23B illustrate the binding and catalytic response to systemic immunization with KLH conjugated E-421-433. FIG. 23A shows the binding and catalytic response in salivary IgA and vaginal IgA. Left axis of each panel shows gp120 cleaving activity (nM/h/μg) of affinity purified IgAs from mice immunized intraperitoneally with E-421-433 KLH conjugate (RIBI as adjuvant). IgA, 3 μg/ml, Bt-gp120 0.1 μM, 16 h. Right axis shows ELISA values for E-421-433 binding of IgA from saliva (1:8) and vaginal wash (1:10) recovered from the same mice detected by peroxidase conjugates of anti-mouse IgA. Arrows indicate injections schedule. FIG. 23B shows the binding response in serum. ELISA values for E-421-433 binding by sera IgG (1:5000), IgM (1:1000) and IgA (1:100) from mice immunized intraperitoneally with E-421-433 KLH conjugate (RIBI as adjuvant) detected by peroxidase conjugates of anti-mouse IgG, IgM, or IgA. Arrows indicate injections schedule.

FIGS. 24A-24C demonstrates stimulation of gp120 binding IgM synthesis by Protein A. FIG. 24A shows induction of gp120-binding IgM in mice stimulated with Protein A. Shown are serum IgM titers in preimmune mice and Protein A-stimulated mice (day 30; 1 mg Protein A i.v. on days indicated by arrows in panel B; 1:1000 dilution) determined using gp120 coated plates (0.2 μg/well). Bound IgM was detected with biotinylated anti-mouse IgM (K-chain specific; 1:1000) and peroxidase-conjugated streptavidin (1:1000). Identical assays without gp120 coating yielded A490 values of 0.046±0.002 (day 0) and 0.027±0.001 (day 30). FIG. 24B is a time course of gp120-binding IgM response in Protein A-stimulated mice. Protein A (1 mg, i.p. in PBS) administered on days indicated by arrows. Shown are ELISA data for sera obtained at various time points. Bound IgG and IgM were detected, respectively, with peroxidase-conjugated goat anti-mouse IgG (Fc specific; 1:500) and goat anti-mouse IgM (_-chain specific; 1:500).

FIGS. 25A-25C demonstrate specificity of gp120-binding IgM induced by protein A stimulation. FIG. 25A shows inhibition of gp120-IgM binding by gp120. Shown are gp120-binding ELISA data in the presence and absence of gp120 (0.5 μM). Sera: day 35, 1:100 dilution in 1% skim milk/PBST. Other conditions are as in FIG. 2B. FIG. 25B shows specific E-421-433 binding by IgM. Shown are streptavidin-peroxidase-stained reducing SDS-gels of IgM incubated with E-gp120₄₂₁₋₄₃₃ (lane 1), a control electrophilic probe containing shuffled sequence of gp120₄₂₁₋₄₃₃ (lane 2) and E-hapten (lane 3). IgM, 56 μg/mL; E-gp120421-433 and control probes, 1 μM; 37° C., 1 h. In addition to the 70-kDa μ-chain band and the 25-kDa κ/λ band, a 50 kDa band was evident in lane 1, identified previously as a μ-chain fragment based on staining with anti-μ antibody (36). FIG. 25C depicts the structures of probes used in FIG. 25BB. LC, aminohexanoyl linker.

FIGS. 26A-26B depict the shared structural basis for B cell superantigenic properties of Staphylococcal Protein A and HIV gp120. FIG. 26A is a schematic representation of the complex between Staphylococcus Protein A domain D (SpA-D) and Fab 2A2 from a human IgM (PDB IDEE). Shown is a zoom-in view of the interface of SpA-D (line ribbon) and Fab 2A2 (solid ribbon). Antibody amino acids reported to interact with SpA [Graille M, et al, 2000] or gp120 [Karray S, et al, 1998] are shown in the Corey, Pauling and Koltun (CPK) style—red CPK residues represent amino acids interacting with both gp120 and SpA-D; blue, amino acids interacting with gp120 only; green, amino acids contacting with SpA-D only. FIG. 26B is a schematic representation of superimposed SpA-D and gp120 residues 421-433. Shown is a gp120 residues 421-433 in helix form (green ribbon; an energy-minimized structure obtained by CHARMm/DS modeling 1.7 (Accerelys)) superimposed on SpA-D helix II (light gray ribbon; extracted from the crystal structure, PDB 1DEE), where amino acids that interact with gp120-contacting amino acids of Fab 2A2 are clustered (shown in ball and stick style; a blue ball represents a nitrogen atom; red, oxygen atom; carbon atoms and bonds are shown in backbone colors). RMSD, 0.88 Å.

FIG. 27 depicts E-Protein A. The E-Protein A stimulant contains the electrophilic phosphonate groups on Lys side chain amino groups.

FIGS. 28A-28B are an example of non-BCR-utilizing polyclonal B cell stimulants. FIG. 28A shows E-LPS. Shown is E-LPS derived from E. coli Re LPS. Two electrophilic groups are placed on the carboxylic group of 2-keto-3-deoxyoctolusonic acid residues. FIG. 28B is E-CpG. Shown is E-CpG derived from CpG ODN2006, TCG TCG TTT TGT CGT TTT GTC GTT linked by phosphorothioate bonds. Electrophilic groups are placed via 4′-aminomethyl-4,5′,8-trimethylpsoralen (AMT), which preferentially react with thymidine (T) residues. The primary site of thymidine-AMT attachment is between the 5,6 double bond of thymidine in CpG and the psoralen 4′,5′ double bonds (arrow a). Further reaction with another thymidine (arrow b) is possible to occur, forming an inter- or intra-strand cross-link. The electrophilic group di(4-nitrophenyl) suberoylamino(4-amidinophenyl)methanephosphonate is linked to the amino group of AMT (T-PsP unit).

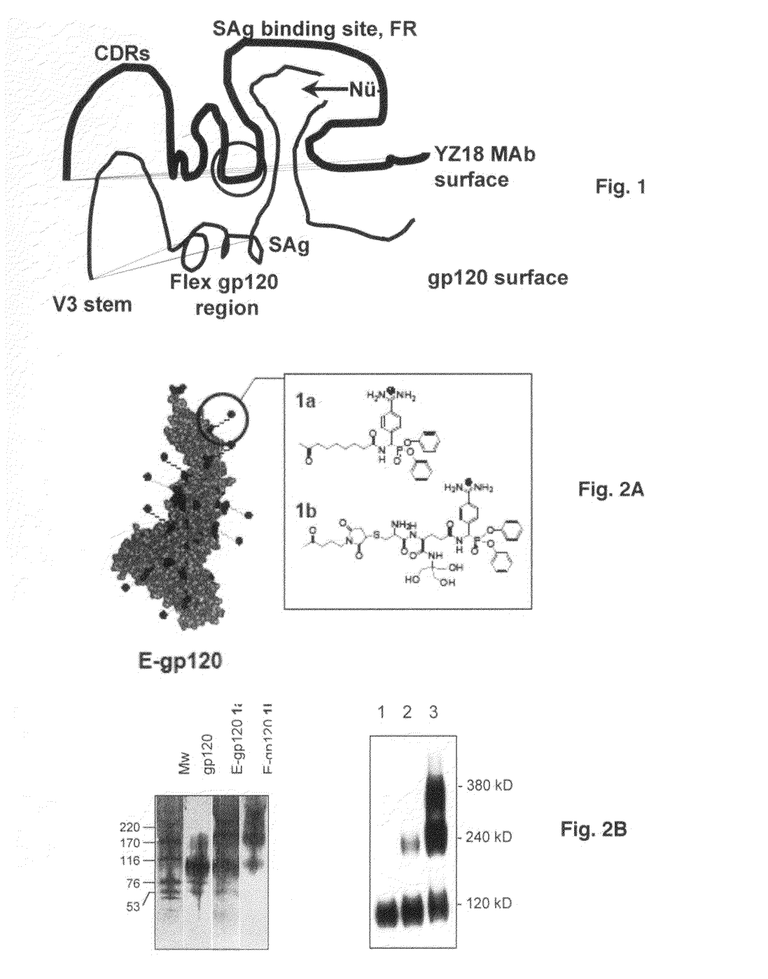

### DETAILED DESCRIPTION OF THE INVENTION

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any compound, composition, or method described herein can be implemented with respect to any other device, compound, composition, or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

As used herein the term “animal” refers to a mammal, preferably a human.

As used herein the term “subject” refers to any recipient of a binary epitope specific antibody administered to treat HIV.

In one embodiment of the present invention, there is provided a polypeptide or electrophilic analog thereof comprising a superantigenic epitope and at least one other epitope effective to induce the production of antibodies with binary specificity for the epitopes on a polypeptide antigen. In a further embodiment one or both epitopes further comprise one or more electrophilic groups therewithin where the electrophilic groups effective to induce synthesis of the binary specific antibodies.

In both embodiments the electrophilic analog may be covalently oligomerized gp120. Also, the polypeptide or electrophilic analog thereof may comprise superantigenic gp120 epitope linked to another gp120 epitope by a peptide linker. Particularly, the superantigen epitope may comprise amino acid residues 421-433 of gp120 and the other epitope may comprise amino acid residues 301-311 of gp120. Also, the peptide linker has a sufficient number of amino acids such that the linker length approximates the distance between the two epitopes in native gp120.

Also in both embodiments one or both of the epitopes comprising the electrophilic analog has the structural formula:

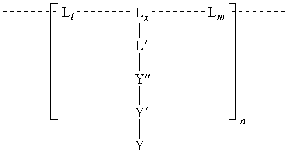

where L₁ . . . Lx . . . Lm are components defining an antigenic determinant; Lx is a component amino acid of the antigenic determinant; L′ is a functional group of Lx; Y″ is a molecule, a covalent bond or a linker; Y′ an optional charged or neutral group; Y is an electrophilic group that reacts covalently with an antibody that binds to said antigenic determinant; n is an integer from 1 to 1000; and m is an integer from 4 to 30. Furthermore, in both embodiments the Y″, Y′ or Y further may comprise a water-binding group as a terminal or internal component. Particularly, the water-binding group binds a metal ion that chelates one or more water molecules. Examples of the metal binding group are -(His)ₙ-, where n=2 or more or -Cys-X-Cys-Cys- or -Cys-X-Cys-, where X is an amino acid residue, ethylene diamine tetraacetic acid or diaminomethyl pyridine. Examples of the metal are the metal is zinc, copper, nickel, cobalt, calcium, or magnesium.

In another embodiment of the present invention there is provided a method for producing binary epitope specific antibodies to a B cell polypeptide antigen, comprising administering one or more of the polypeptide constructs of described supra or one or more of an electrophilic polypeptide analog thereof to a living animal. Further to this embodiment the method comprises administering one or more immunological adjuvants effective to stimulate T-cell independent or T-cell dependent B cell antibody production to the living animal.

In both embodiments the polypeptide antigen may be HIV gp120, Tat, Protein A, or Protein L. Also, the antibodies may recognize amino acid residues 421-433 and residues 301-311 of HIV gp120 epitopes. In addition, the binary epitope specific antibodies catalyze the hydrolysis of the native polypeptide antigen or covalently bind the native polypeptide antigen, e.g., gp120 expressed on the surface of HIV, thereby neutralizing HIV.

In a related embodiment there is provided an antibody with binary specificity to a B cell polypeptide antigen produced by the method described supra. In another related embodiment there is provided a method for treating HIV in a subject, comprising administering an immunologically effective amount of the antibody described supra to the subject.

In yet another embodiment of the present invention there is provided a method for increasing production of antibodies recognizing the B cell superantigenic site of HIV gp120, comprising administering one or both of a polyclonal B cell stimulant or an electrophilic analog thereof to an living animal. Further to this embodiment the method comprises administering one or more immunological adjuvants effective to stimulate T cell-independent B cell antibody production to the living animal.

In both embodiments the polyclonal B cell stimulant may be one or more of pokeweed mitogen, lipopolysaccharide, phytohemagglutinin, or CpG. Also, the polyclonal B cell stimulant may be Staphylococcal Protein A. Particularly, the polyclonal B cell stimulant may be a superantigenic domain of Protein A, an oligomer of the superantigenic domain of Protein A or Protein A labeled with iodine. In addition the stimulant further may comprise electrophilic groups that stimulate production of antibodies effective to catalyze the hydrolysis of gp120 or to covalently bind gp120. Furthermore, the electrophilic analog of the polyclonal B cell stimulant having one or more epitopes may have the structure as described supra.

In a related embodiment there is provided an antibody recognizing the B cell superantigenic site of HIV gp120 produced by the method described supra. In another related embodiment there is provided a method for treating HIV in a subject, comprising administering an immunologically effective amount of the antibody described supra to the subject.

In yet another embodiment of the present invention there is provided a method for stimulating increased production of antibodies comprising administering to a living animal a combination of a dual epitope polypeptide and electrophilic analog thereof with an electrophilic analog of a polyclonal B cell stimulant.

In yet another embodiment of the present invention there is provided a method for isolating an individual antibody or antibody fragment thereof having a unique sequence and binary epitope specificity from an antibody repertoire, comprising displaying the antibody repertoire on the surface of phage particles; and screening the antibody repertoire with a polypeptide or a polypeptide electrophilic analog thereof, wherein an antibody or antibody fragment thereof reacting with the polypeptide or the electrophilic analogs thereof thereby isolates the binary epitope specific antibody or antibody fragment thereof from the antibody repertoire.

In yet another embodiment of the present invention there are provided electrophilic analogs of a lipid, a polysaccharide or a lipopolysaccharide having the general polypeptide electrophilic analog structure as described supra where L₁ . . . Lx . . . Lm are components defining a receptor binding determinant; where Lx is a component sugar or lipid of the receptor binding determinant; L′ is a functional group of Lx; Y″ is a molecule, a covalent bond or a linker; Y′ an optional charged or neutral group; Y is an electrophilic group that reacts covalently with a cellular receptor that binds to said receptor binding determinant, or an acyl group; wherein, optionally, Y″, Y′ or Y comprises a water-binding group as a terminal or internal component; n is an integer from 1 to 1000; and m is from 1 to 1000.

In yet another embodiment of the present invention there is provided electrophilic analogs of a nucleotide having the general polypeptide electrophilic analog structure as described supra where L₁ . . . Lx . . . Lm are L₁ . . . Lx . . . Lm are nucleotide components defining a receptor binding determinant; Lx is a component nucleotide of the receptor binding determinant; L′ is a functional group of Lx; Y″ is a molecule, a covalent bond or a linker; Y′ an optional charged or neutral group; Y is an electrophilic group that reacts covalently with a cellular receptor that binds to said receptor binding determinant, wherein, optionally, Y″, Y′ or Y contains a water-binding group as a terminal or internal component; n is an integer from 1 to 1000; and m is from 1 to 1000.

The present invention describes means to overcome the barriers that limit adaptive antibody responses capable of protecting against the effects of superantigenic polypeptides, for example, but not limited to, the HIV coat protein gp120. The superantigenic site of HIV gp120 is relatively conserved and contributes amino acids that are critical for virus binding to CD4 receptors on host cells and the infection thereof. Consequently, it is contemplated that antibodies to this site are effective to neutralize diverse HIV isolates belonging to different clades.

The present invention also describes a hitherto unsuspected property of certain antibodies, i.e., the ability to recognize two distinct epitopes of gp120. This antibody property, designated ‘binary epitope specificity’, results in neutralization of the HIV infection as tested in tissue culture. In addition, the ability of various polypeptide constructs to induce the synthesis of antibodies with the binary epitope specificity and HIV neutralizing activity is disclosed. It is demonstrated herein how inclusion of two epitopes in the polypeptide construct can stimulate the production by B cells of antibodies that recognize one of the epitopes by interactions at the CDRs, and of the second epitope, by interactions at the FRs. Recognition of the former epitope in the present invention is driven by conventional B cell clonal selection processes. The cellular proliferative signal generated by interaction at the CDRs is sufficient to overcome the negative effect of interactions of the second, superantigenic epitope at the FRs, resulting in production of antibodies that express binary epitope specificity. These considerations are important for designing an effective HIV vaccine that induces protective antibodies to the virus with binary epitope specificity.

Also provided herein are methods to generate antibodies with binary epitope specificity with increased virus neutralizing activity. The increase in virus neutralizing activity is derived from the chemical reactivity of the antibodies. Conventional antibodies bind antigens reversibly by noncovalent mechanisms. Like conventional antibodies, chemically reactive antibodies initially recognize the antigen by noncovalent means. However, nucleophilic sites located in the V domains of chemically reactive antibodies can then proceed to recognize electrophilic reaction centers in the antigen.

This has two consequences, formation of irreversible immune complexes with covalent nucleophile-electrophile pairing, and, if a water molecule is available at the reaction center, the hydrolysis of the antigen by the antibody. The irreversible binding property enhances antibody potency, because it precludes regeneration of active antigen by dissociation of the immune complexes. Similarly, the catalytic hydrolysis step results in irreversible antigen inactivation, as the antigen fragments do not usually express biological activity similar to the parent antigen. The invention discloses dual epitope polypeptide constructs in which strongly electrophilic groups are incorporated by chemical means. The electrophilic groups induce adaptive strengthening of antibody nucleophilic reactivity, and thus increase the ability of the polypeptide constructs to induce the synthesis of protective antibodies with binary epitope specificity.

Thus, the dual epitope polypeptide constructs provided herein may be employed as a prophylactic or immunotherapeutic vaccine. In the case of HIV, for example, a polypeptide or an electrophilic analog thereof containing at least the two epitopes of gp120 may be administered repeatedly to a living organism to induce the synthesis of protective antibodies, including antibodies produced by memory B cells.

Particularly, the electrophilic analog of a dual epitope polypeptide where one or both of the epitopes may have the structure

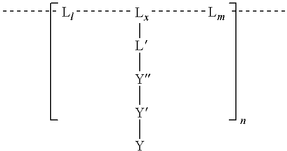

In this structure L₁ . . . Lx . . . Lm are components defining an antigenic determinant where Lx is a component amino acid of the antigenic determinant, L′ is a functional group of Lx, Y″ is a molecule, a covalent bond or a linker, Y′ an optional charged or neutral group, Y is an electrophilic group that reacts covalently with an antibody that binds to said antigenic determinant, n is an integer from 1 to 1000, and m is an integer from 4 to 30. Optionally, any of Y″, Y′ or Y further may comprise a water-binding group as a terminal or internal component effective to bind a water molecule(s) chelating metal, such as, zinc, copper, nickel, cobalt, or magnesium. The metal binding groups may be a poly histidine, -(His)ₙ-, where n=2 or more. Alternatively, the metal binding groups may be -Cys-X-Cys-Cys- or -Cys-X-Cys-, where X is an amino acid residue, ethylene diamine tetraacetic acid (EDTA) or diaminomethyl pyridine. Also, provided is an electrophilic analog of a polysaccharide or a lipopolysaccharide having the same general structure where L₁ . . . Lx . . . Lm are components defining a receptor binding determinant, Lx is a component sugar or lipid of the receptor binding determinant and m is an integer from 1 to 1000. In addition, provided is an electrophilic analog of an oligonucleotide where L₁ . . . Lx . . . Lm are nucleotide components defining a receptor binding determinant, Lx is a component nucleotide of the receptor binding determinant and m is an integer from 1 to 1000.

The administration of the vaccine can be conducted by any route that is effective, for example, the intramuscular, intravenous, intraperitoneal and mucosal routes. HIV is often transmitted across the mucosal route. Therefore, mucosal vaccination is a preferred embodiment of the invention, as this route maximizes the production of protective IgA antibodies at mucosal surfaces. Also, the dose or dosage of antibodies administered is easily determined by one of ordinary skill in the art. Additionally, it is well established to administer the antibodies in an immunogenic composition comprising adjuvants and/or diluents known in the art.

The present invention also demonstrates the unexpected ability of another superantigen, Protein A, to stimulate the synthesis of antibodies to HIV gp120. Although the sequences of the superantigenic sites of Protein A and gp120 are divergent, there is sufficient conformational similarity that cross-reactive antibody synthesis is permissible. It is contemplated that the anti-gp120 antibody production is permissible regardless of whether synthesis of antibodies to Protein A itself occurs. Based on this unexpected finding, it can be anticipated that administration of Protein A may be useful to direct the initial B cell response towards the superantigenic site of gp120. Also, the administration of Protein A can be combined with gp120 or the foregoing dual epitope polypeptide constructs to overcome the natural barriers limiting antibody synthesis to the gp120 superantigenic site.

In addition, the present invention provides methods using appropriate polyclonal B cell stimulators or activators alone or in conjunction with the dual epitope polypeptide constructs for induction of the protective antibodies to HIV. Such activators interact with non-BCR receptors to induce activation and proliferation of multiple B cell subpopulations regardless of their antigenic specificity. Non-limiting examples are lipopolysaccharide, heat labile E. coli enterotoxin, cholera toxin B, various interleukins, cytokines, CpG and the like. As synthesis of neutralizing antibodies to the gp120 superantigenic site is a B cell property that does not require their adaptive maturation driven by the superantigen, polyclonal B cell activation may result in enhanced protective antibody production. When polyclonal B cell activator is administered combined with a suitable immunogen, it is contemplated that the adaptive immune process may improve antibody specificity and antibody chemical reactivity.

Synthesis of antibodies with binary epitope specificity was observed in response to immunization of experimental animals with an electrophilic analog of full-length gp120 in the present invention. Monoclonal antibodies and fragments thereof can be prepared readily from the spleen or other lymphoid tissues of experimental animals by routine methods well known and available in the art, e.g., hybridoma technology and phage display technology. Similarly monoclonal antibodies and fragments thereof can be prepared from the B cells of humans immunized with the dual epitope polypeptide constructs or electrophilic gp120 provided herein. Thus, the present invention provides methods for passive immunization of humans with HIV infection using monoclonal antibodies or fragments thereof with binary epitope specificity and HIV neutralizing activity.

The present invention further demonstrates that full-length gp120 expressed on the surface of the virus induces the synthesis of protective antibodies with binary epitope specificity, albeit only rarely and preferentially in HIV infected individuals with a natural immune resistance to HIV. It also is contemplated that humans with prolonged HIV infection and no progression to acquired immune deficiency syndrome (AIDS) produce increased catalytic antibodies to the superantigenic site of gp120. Furthermore, as presented herein, antibodies with binary epitope specificity are isolated from such infected subjects using hybridoma methods, immortalization of B cells using Epstein Barr virus and construction of libraries of antibodies or their fragments from the expressed B cell repertoire. These methods are routine in the art. In these methods, the dual reactive polypeptide constructs are utilized to identify the subpopulation of B cells or antibodies displayed on a suitable vector, e.g., a phage display vector, that express the binary epitope specificity. It is contemplated that such antibodies are useful for passive immunotherapy of humans with HIV infection.

The following example(s) are given for the purpose of illustrating various embodiments of the invention and are not meant to limit the present invention in any fashion.

### EXAMPLE 1

### Binary Epitope Specificity of Nucleophilic Mabs Raised by Immunization with Electrophilic Analogs of HIV gp120

The HIV envelope protein gp120 is a target for experimental vaccines that attempt to induce the synthesis of neutralizing antibodies (Abs) (1, 2). Most Abs induced by immunization with gp120 are directed to the highly mutable regions of the protein, particularly the third variable domain (V3 domain) (3). These Abs neutralize the infecting viral strain (4). However, the gp120 V domains mutate over the course of infection, resulting in viral escape mutants refractory to neutralization by the Abs (5). Moreover, the gp120 V domains of various HIV strains responsible for the pandemic in different parts of the world are highly divergent, and Abs induced by immunization with gp120 usually fail to neutralize heterologous HIV strains (6). This has lead to the search for structurally conserved neutralized epitopes expressed by gp120. Candidate epitopes are the comparatively conserved regions of gp120 involved in HIV binding to host cell receptors, i.e., CD4 and chemokine receptors. Regrettably, these epitopes are poorly immunogenic. Rare monoclonal Abs (MAbs) directed to regions close to the CD4 and chemokine receptor binding sites of gp120 have been identified using complex experimental protocols (7-9). These MAbs neutralize many but not all clade B HIV-1 strains (10).

The CD4 binding site (CD4bs) is thought to be a discontinuous determinant composed of residues 256, 257, 368-370, 421-427 and 457 (11-14). The CD4bs is suggested to undergo a conformational transition(s) when the gp120 trimer expressed on the viral surface is shed as soluble monomers, and Abs to the monomer CD4bs are often poorly reactive with the trimer CD4bs (15). Moreover, the 421-427 peptide region of the CD4bs is an important component of the B lymphocyte superantigen site of gp120 (16) (gp120_{SAg}; defined as a site to which Abs are present in the preimmune repertoire without the requirement for adaptive sequence diversification of the Ab variable domains). As in the case of other B cell superantigens, gp120_{SAg} binding to immunoglobulin (Ig) expressed as part of the B cell receptors (BCRs) is thought to induce B cell apoptosis (17-19), and there is no evidence for adaptive amplification of gp120_{SAg} binding Abs in HIV infected subjects. To the contrary, HIV infected subjects express diminished serum levels of VH3+ Igs, the VH family thought to bind gp120_{SAg} preferentially (20). Despite evident difficulties, powerful arguments supporting targeting of the CD4bs remain. These are: (a) The binding of gp120_{SAg} by IgG class Abs in humans without infection is correlated with the reduced incidence of subsequent HIV infection (21); (b) Moderate affinity IgA and IgM class Abs in uninfected humans catalyze the hydrolysis of gp120, with specificity of the reaction deriving from recognition of the 421-433 region (22, 23), and the IgA class Abs were recently observed to neutralize HIV (23), and (c) Patients with systemic lupus erythematosus, an autoimmune disease that is coexistent with HIV infection only rarely (24), produce Abs to the 421-433 synthetic peptide at increased levels (25), and a 421-433 recognizing single chain Fv fragment (variable domains of the light and heavy chain Ab subunits tethered by a linker peptide) isolated from a lupus Ab library neutralizes diverse primary HIV strains in tissue culture (26).

Along with their epitope specificity determined by noncovalent binding forces, Abs bring nucleophiles located within their combining sites into proximity with electrophilic groups in the antigens (27, 28). Enzymes utilize similar nucleophile-electrophile interactions to form covalent reaction intermediates with substrates that are subsequently hydrolyzed by water attack on the acyl-enzyme complex (29). Hypothesizing that adaptive immune processes can strengthen Ab nucleophilic reactivity, we studied IgG class MAbs raised by immunization with gp120 containing electrophilic phosphonate diesters (E-gp120) (30). Two types of MAb chemical reactivities attributable to induction of the enhanced nucleophilic reactivity were deduced, gp120-specific proteolysis (30) and the formation of unusually stable immune complexes with gp120 displaying covalent character (31). Here, we describe the binary epitope specificity of the nucleophilic MAbs raised by immunization with E-gp120. Unlike conventional MAbs to gp120, the anti-E-gp120 MAbs recognized two spatially distant gp120 epitopes located in the stem of the V3 loop and in the superantigenic region overlapping with conserved CD4bs. Despite the heterologous nature of the clade B E-gp120 immunogen, the binary epitope-reactive MAbs neutralized primary HIV belonging to clades A and C. The properties of the MAbs suggest novel ways to induce neutralizing Abs to the CD4bs of gp120.

### E-gp120 Immunogens

E-gp120 immunogens were obtained by placing electrophilic phosphonate diphenyl diester groups at Lys side chains of the recombinant gp120 (MN strain) using two alternate linkers (FIG. 2A). Close to the phosphonate is the 4-amidinophenyl substituent, a positively charged group that mimics the Lys/Arg flanking residue recognition capability of the naturally occurring Ab nucleophilic sites. Multiple phosphonates were available per molecule of gp120 (3046 phoshphonates/gp120), allowing presentation of the electrophiles in conjunction with diverse antigenic epitopes. The phosphonate mimics the carbonyl group at the peptide bond susceptible to enzymatic nucleophilic attack. Denaturing electrophoresis of E-gp120 revealed the presence of covalent E-gp120 dimers and trimers along with the monomer (FIG. 2B). The oligomerization is not an indiscriminate reaction, as similar electrophilic derivatives of other proteins, e.g., the epidermal growth factor receptor, did not oligomerize detectably.

### MAb Preparation

Three separate immunizations of mice were conducted using two alternate E-gp120 immunogens (30-46 phosphonates/gp120; monomer and oligomer proportions in the different preparations were, respectively, 20-50% and 50-80%) and hybridomas were prepared. Screening of MAbs in culture supernatants was by a covalent ELISA protocol, in which Abs bound noncovalently to immobilized immunogen were removed by washing with 2% SDS prior to detection of immune complexes. Thirty nine of 705 IgG-secreting hybridoma wells and 15 of 373 IgM-secreting hybridoma wells displayed detectable SDS-resistant binding to the immobilized E-gp120 (A490>0.2; range 0.2-2.9; background values with medium<0.1).

### HIV Neutralization

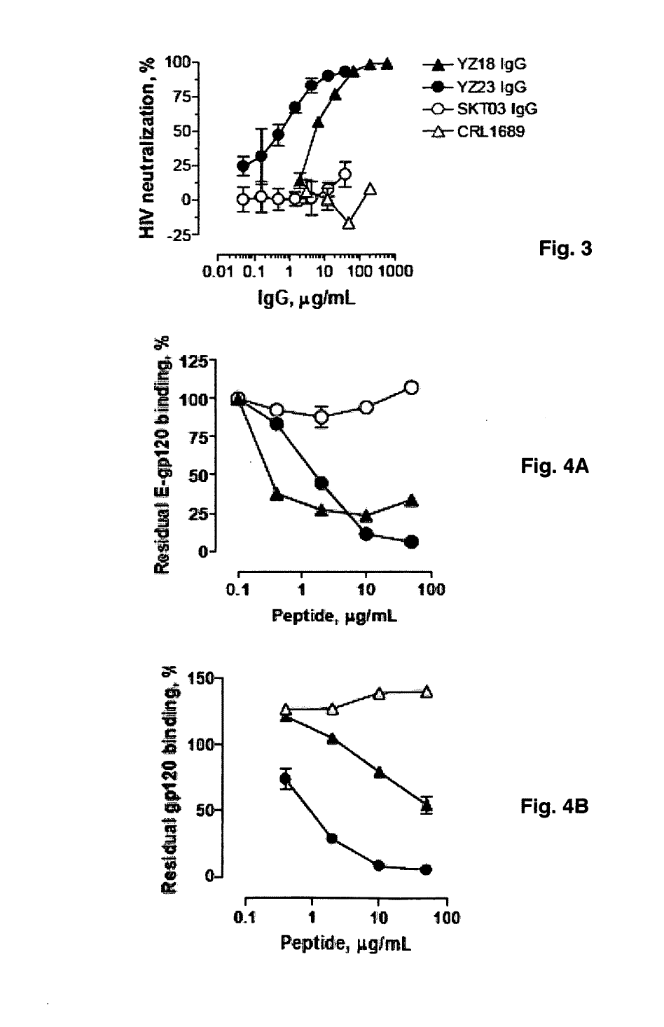

MAbs were assayed for neutralization of primary HIV-1 isolates using peripheral blood mononuclear cells (PBMCs) hosts (p24 enzyme immunoassay). The HIV-1 isolates studied were R5 clade B (SF162, JR-CSF, W61D), R5 clade C strains (BR004, ZA009) and an X4 clade D strain (UG046). Controls included the equivalently purified irrelevant MAb CRL1689 with the same isotype as anti-E-gp120 MAbs YZ18, YZ22 and YZ23 (IgG2a, _). IgG was purified by protein G-Sepharose chromatography. The anti-E-gp120 MAbs were not cytotoxic for PBMCs, determined by viability assays in the absence of HIV. Seventeen IgG MAbs with E-gp120 binding activity were tested for neutralization of HIV strain ZA009. Ten IgG MAbs displayed dose-dependent and reproducible HIV neutralizing activity (examples in FIG. 3). In contrast, none of the 9 E-gp120 binding IgM MAbs neutralized the virus. Two IgG clones were studied further (clones YZ18 and YZ23). Both IgGs neutralized all of the HIV strains studied except the clade D strain (Table 1). Neutralization of clade C strains is interesting because MAbs described previously neutralize clade C strains poorly or not at all (10). With a clade B strain, the potency of IgG YZ23 was comparable or superior to the reference MAb, clone b12. Both IgGs also neutralized the SHIV strain (SF162P3), and the neutralizing activity was comparable in the absence of cervicovaginal lavage fluid from rhesus macaques (20% v/v; not shown).

TABLE 1
Neutralizing activity of MAbs YZ18 and YZ23.
PHA-stimulated PBMC hosts. Neutralization computed
as % decrease of p24 or p27 (SHIV strain) concentrations
in MAb containing wells. Data are from MAb dose-response curves.

| | IC₈₀, _g/mL | | |
|---|---|---|---|
| Clade_Strain | MAb YZ23 | MAb YZ18 | MAb b12 |
| B_SF162 | 4.8 | 2.7 | 14.1 |
| B_JR-CSF | 30 | NT | NT |
| B_W61D | 25 | NT | NT |
| C_BR004 | 5.3 | 6.5 | >20 |
| C_ZA009 | 15 | 19 | >20 |
| D_UG046 | >200 | NT | NT |
| SHIV (SF162P3) | 5.0 | <1.9 | NT |

### Binary Epitope Reactivity

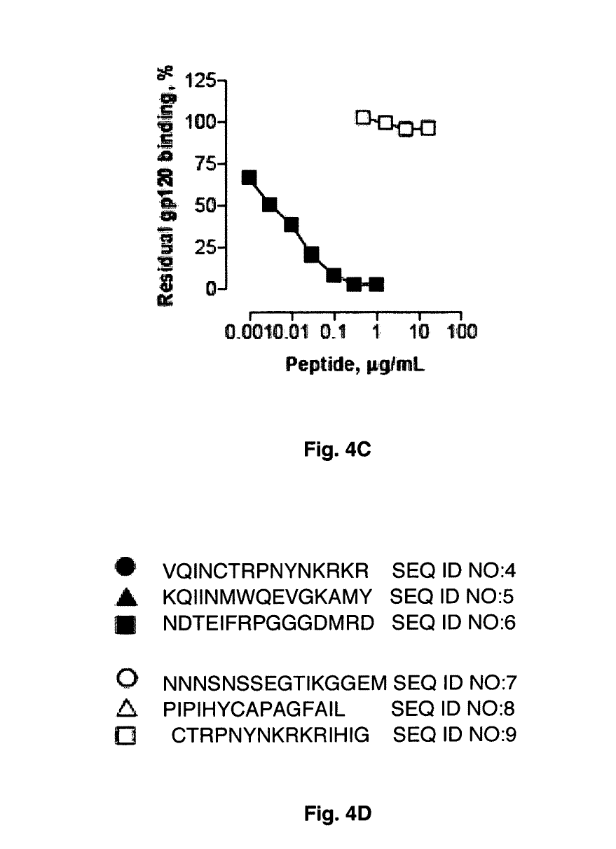

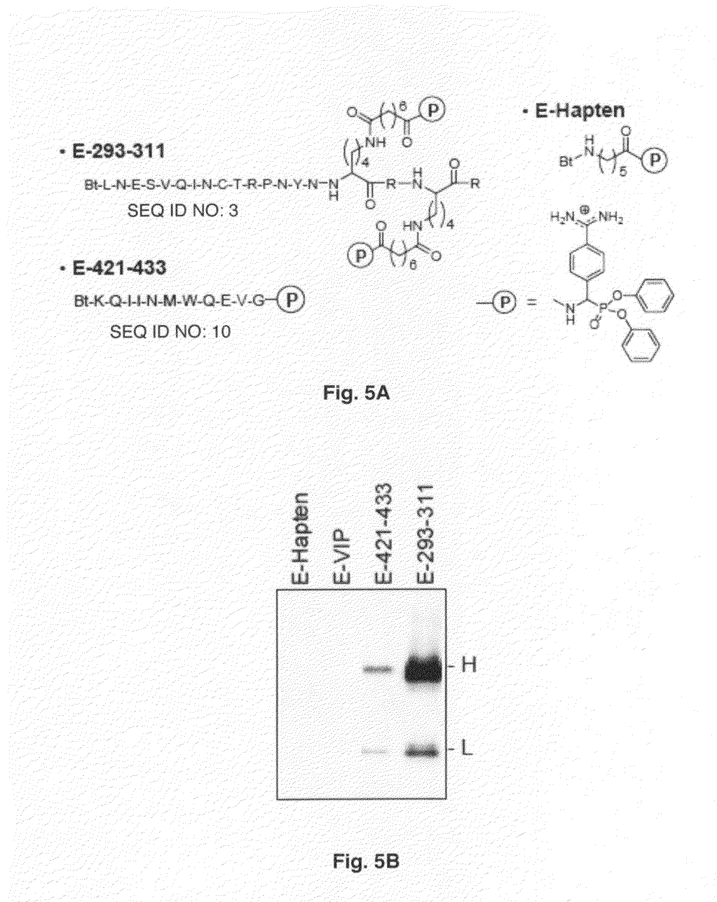

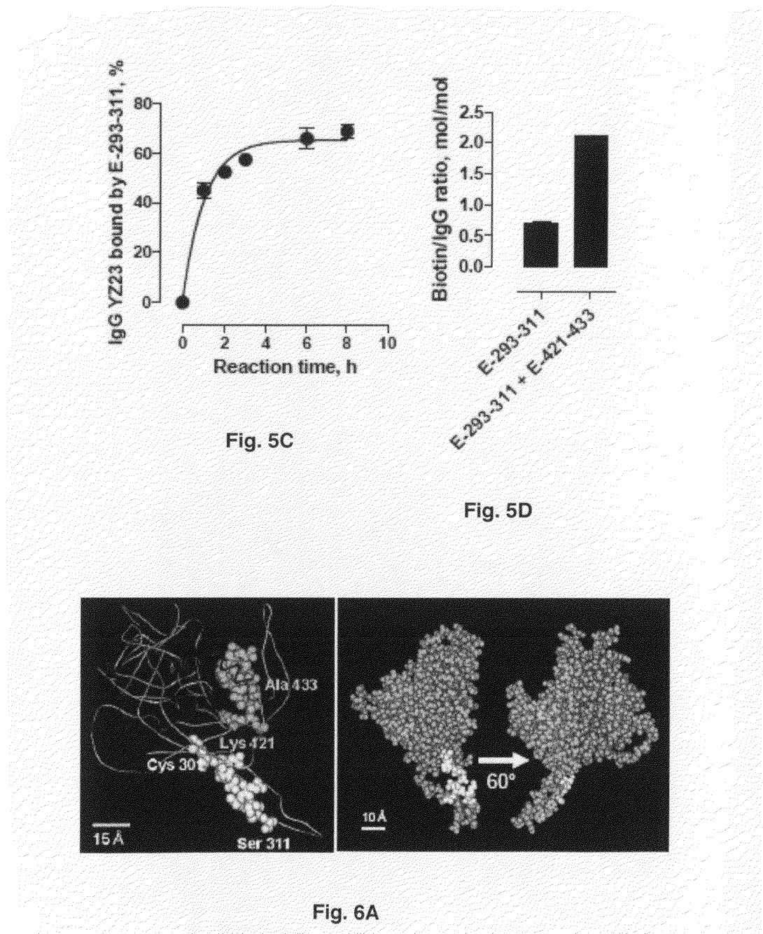

The epitope reactivity of two neutralizing IgGs (YZ18 and YZ23) and one non-neutralizing IgG (SK-T03) was tested using 15-mer peptides corresponding to gp120 residues 27-512 as competitive inhibitors. The ELISA plates contained immobilized gp120 (IgG YZ23, SK-T03) or E-gp120 (IgG YZ18 binding; to avoid loss of gp120 by catalytic cleavage; see description of proteolytic activity below). Dose-dependent inhibition of binding of IgG YZ18 and YZ23 by two peptide regions was observed, residues 297-315 and 417-435 (FIG. 4). The non-neutralizing clone SKT03 displayed a differing epitope reactivity. Only one peptide, residues 465-479, inhibited the IgG-gp120 binding competitively. For both neutralizing IgGs, peptides 297-311 and 301-315 were equipotent inhibitors, suggesting the overlapping region 301-311 as a recognition element. Similarly, peptide 417-431 and 421-435 inhibited the binding with near equivalent potency, suggesting 421-431 as the second important recognition element. The binary epitope reactivity was confirmed by electrophoresis and ELISA assays using electrophilic derivatives of biotinylated peptides corresponding to residues 293-311 (E-293-311) and 421-433 (E421-433) (FIG. 5A). These electrophilic analogs are sensitive probes for specific antigen-Ab binding, determined from the formation of covalent immune complexes in SDS-PAGE. The structure and reactivity of E-421-433 are published (28). E-293-311 was obtained similarly by placing phosphonate groups at the side chains of Lys308 and Lys310. Both E-peptides formed complexes with IgGs YZ18 and YZ23 at levels above the control irrelevant electrophilic peptide probe E-VIP (27) (example with YZ18 shown in FIG. 5B). Time course of E-293-311 binding by IgG YZ23 was in agreement with the one-site exponential binding model (% Binding=100(1−e^{-k·t}); k, the first order rate constant) with the 50% saturation time of 43 min (FIG. 5C, left panel). Following the near saturation of E-293-311 binding site, IgG YZ23 was still capable of binding to the equimolar amount of E421-433 (FIG. 5C, right panel), indicating that the independent subsites of the MAb are responsible for recognition of 293-311 and 421-433, and simultaneous occupancy of two binding sites in the MAb.

### Structural Basis for Dual Epitope Recognition

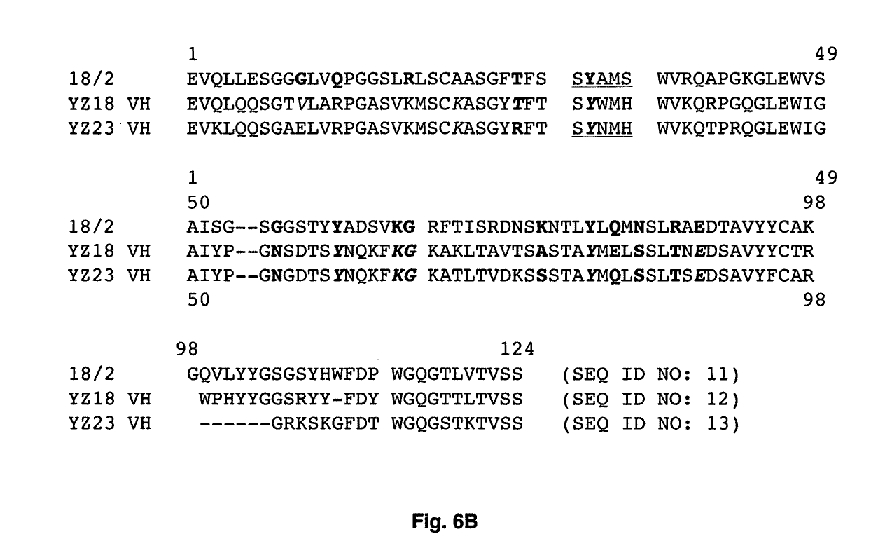

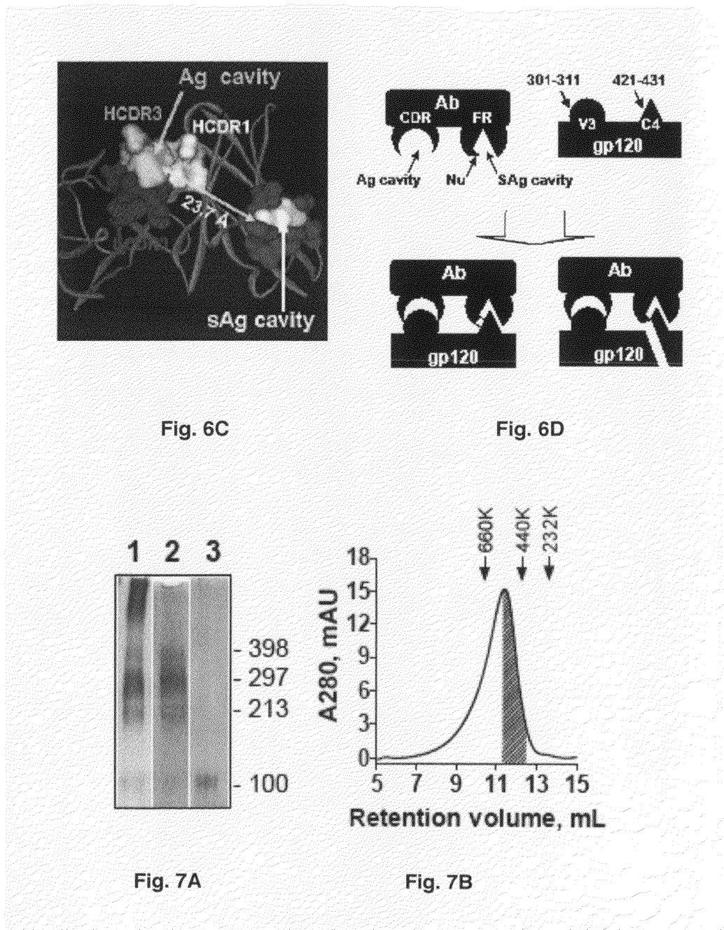

Residues 301-311 and 421-433 are located at considerable distance in the 3-dimensional structural models of monomer human gp120 (FIG. 6A) and trimer simian gp120 (not shown) deduced from X-ray crystallography. The two peptide regions are unlikely, therefore, to form a single, contiguous epitope.

The V domains of IgGs YZ18 and YZ23 were sequenced using RT-PCR amplified cDNA from the hybridoma cells. Nine replacement mutations were evident in the V_{H} and V_{L} gene regions of each IgG, and numerous additions/deletions were observed at the V-D-J and V-J junctions (compared to the germline genes). Interestingly, the distributions of V_{H} mutations was skewed in favor of the FRs, and all of the FR mutations were located at positions previously suggested (32) to be important in gp120 SAg binding (FIG. 6B). We leave open the possibility, therefore, that FR mutations can improve the recognition of gp120.

We have solved the crystal structure of the Fab fragment of MAb YZ18 (FIG. 6C) at 2.4 Å resolution (R factor 0.33). Following are the conclusions: (a) several diads containing potential activated nucleophiles were evident (a Ser, Thr or Tyr hydroxyl that can approach a general base within the distance range of a hydrogen bond; a cut-off of 3 Å was employed here); (b) One diad contained a framework amino acid suggested to contribute in Ab binding of the gp120 SAg site (diad V_{H} Ser84 O_-Ser84 backbone O, internuclear distance 2.47 Å); and (c) The surface features of the combining site are consistent with the dual recognition model, i.e., the cavity formed by the CDRs is flanked by another, shallower cavity composed in part by certain amino acids previously suggested to contribute in binding the gp120 SAg site (33). These findings suggest a model of gp120 recognition in which residues 301-311 are recognized at the CDRs and residues 421-433, mainly at the FRs (FIG. 6D).

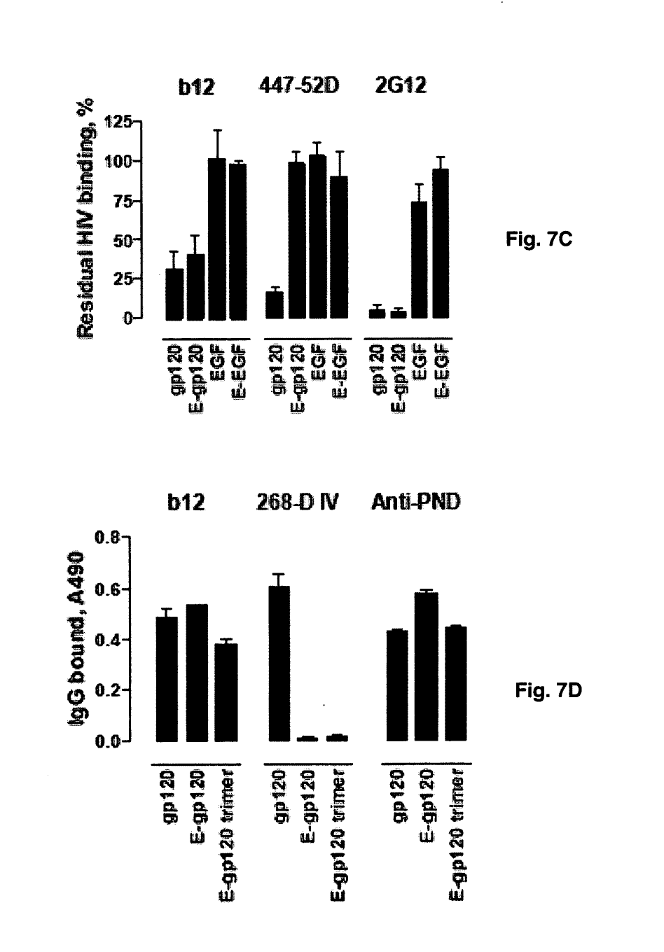

As described above, E-gp120 immunogens contain covalent oligomers (FIG. 2B). The E-gp120 oligomers were stable to boiling for 10 min and gel filtration chromatography (FIG. 7A). We have reported previously that gp120 contains an activated nucleophilic residue covalently reactive with electrophilic hapten (34). The likely reaction underlying covalent oligomerization, therefore, is the covalent binding between a phosphonate diester moiety and a naturally occurring nucleophilic amino acid of gp120. gp120 that had not been derivatized with phosphonate groups did not form covalent oligomers. The E-gp120 oligomerization may be a factor responsible for unique epitope characteristics of the anti-E-gp120 MAbs. We studied the expression of certain known neutralization-relevant epitopes in E-gp120. This was done by a competition ELISA method using MAbs directed to a CD4 binding site epitope (b12; ref (7)), a conformational carbohydrate-dependent epitope (2G12; ref (35, 36)), and a conserved V3 apex epitope (447-52D; ref (37)). Binding of intact HIV by MAb b12 and 2G12, but the anti-V3 MAb 447-52D, was inhibited by E-gp120 (FIG. 7B), suggesting that the V3 apex epitope (GPGR) is not displayed by E-gp120. This was confirmed by direct binding ELISA in which gp120 or E-gp120 was immobilized. E-gp120 was not bound by MAb 268-D IV (epitope, HIGPGR) at a concentration yielding readily detectable gp120 binding (FIG. 7C).

Trimers of E-gp120 have been enriched by gel filtration for further structural studies, e.g., by crystallization to reveal the relative spatial orientation of the two epitopes responsible for the binary epitope specificity.

### Dual Epitope Recognition as a Protective Vaccine Modality

We analyzed the binary epitope specificity of a panel of anti-E-gp120 MAbs consisting of 10 HIV-neutralizing MAbs and 7 MAbs devoid of neutralizing activity. As in FIG. 5B, MAbs were allowed to react with E-293-311 and E-421-433, and the MAb-peptide adducts detected by SDS-electrophoresis followed by streptavidin-peroxidase staining of the blots. Electrophilic probes containing irrelevant peptide sequences (VIP and scrambled gp120 421-433 peptide) were used as controls. Of the 17 anti-E-gp120 MAbs studied, 10 displayed detectable binding to both peptides. Of the 10 dual binding MAbs, 6 displayed HIV strain ZA009 neutralizing activity (IC₅₀<30 μg/ml; FIG. 8). As conventional MAbs neutralize heterologous HIV strains only rarely, it is evident that the binary epitope specificity enhances the likelihood of HIV neutralization.

Using a competition ELISA, we examined the possibility that our neutralizing IgGs bind epitopes similar to certain reference neutralizing MAbs (CD4bs binding MAbs b12 and F105; CD4 inducible-epitope binding MAbs 17b and 48d, binding studied in the presence of soluble CD4; a carbohydrate-dependent epitope binding MAb 2G12). IgG YZ18 and YZ23 did not interfere with gp120 binding by the reference MAbs, indicating that the binary epitope specificity is a unique property of our MAbs.

Conservation of the sequence of residues 421-433 in HIV exceeds 90% (Table 2; all strains available in the Los Alamos database; clades A, B, C, D, F, G, and the CR form). Except for clade D, conservation of residues 301-311 exceeds 86%. This suggests the potential basis for the neutralization resistance of the clade D strain in Table 1 (this strain possesses only 45% identity with the consensus 301-311 sequence).

TABLE 2
Percent conservation of residues 301-311 and 421-433.
Number of HIV strains analyzed, 550; sequences taken from
Los Alamos Database. For each strain, the number of identities
with the consensus residues in the 301-311 epitope
(CTRPNNNTRKS) and the 421-433 epitope
(K-Q-I-I/V-N-M-W-Q-E/R/G-V-G-K/Q/R-A) were counted.
% identities were calculated as 100 × (number of identities)/total
number of residues in the peptide.

% Identity (mean ± SD)

| Clade^{a} | 301-311 | 421-433 |
|---|---|---|
| A (54) | 88 ± 11 | 93 ± 6 |
| B (155) | 90 ± 11 | 95 ± 6 |
| C (111) | 92 ± 7 | 97 ± 5 |
| D (20) | 62 ± 14 | 96 ± 11 |
| F (10) | 92 ± 9 | 93 ± 8 |
| G (11) | 91 ± 7 | 90 ± 4 |
| CRF (189) | 86 ± 15 | 94 ± 7 |

^{a}Numbers in parentheses are the number of strains analyzed.

### Irreversible Binding and Proteolytic Activities

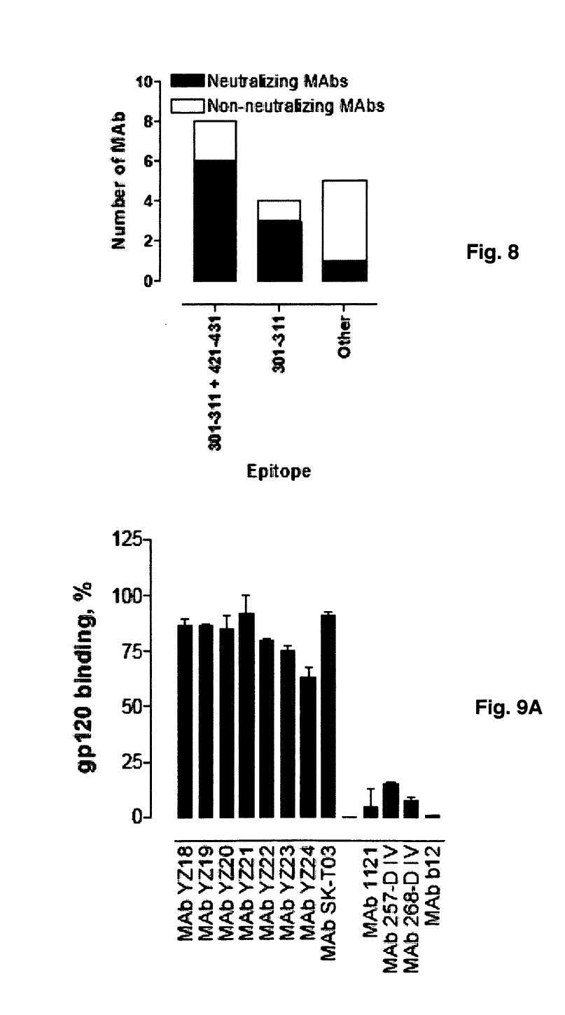

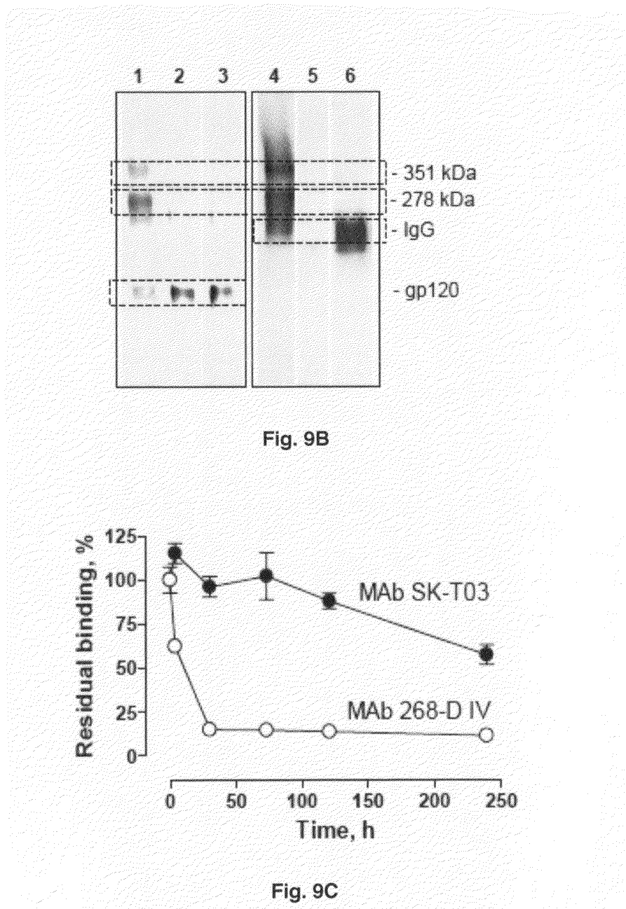

All seventeen IgGs with SDS-resistant E-gp120 binding activity tested also displayed SDS-resistant binding to immobilized gp120 devoid of phosphonate moieties (FIG. 9A shows an example experiment using 8 IgGs). The SDS treatment caused near-complete removal of control anti-gp120 IgGs (directed to V3 domain). Complexes of IgG and gp120 were also evident by SDS-electophoresis (FIG. 9B). Fab fragments also formed SDS-resistant complexes, ruling out an avidity-related artefact. The dissociation in a non-denaturing solution was studied by distinguishing between the dissociable and poorly-dissociable states using the following peptides as competitive inhibitors: MAb YZ23, peptide 297-311; MAb SK-T03, peptide 465-479. Inclusion of excess competitor peptide in the reaction mixture at time zero inhibited the IgG binding to gp120 nearly completely. Addition of competitor peptide at various time points after initiating the incubation with gp120 resulted in progressively decreasing levels of dissociation of the complexes, reflecting formation of the poorly-dissociable complexes as a function of time. The second-order rate constants for the irreversible reaction of MAbs YZ23 and SK-T03 were, respectively, 2.1×10⁶ and 2.1×10⁷ M⁻¹ min⁻¹. The dissociation rate was studied by mixing biotinylated gp120 and IgG, capture of the immune complexes on protein G beads followed by incubation of the beads for varying lengths of time in diluent containing excess peptide competitor (FIG. 9C). Rapid and near-complete dissociation of gp120 from the control MAb (268-DIV) was evident (t_{—} 4.8 hours). Dissociation of complexes formed by IgG SK-T03 was very slow and >50% of the immune complexes remained intact at the final time point examined (10 days; nominal t_{—} 18.5 days; in comparison, t_ for the biotin-streptavidin complex is 1.4-3.3 days).

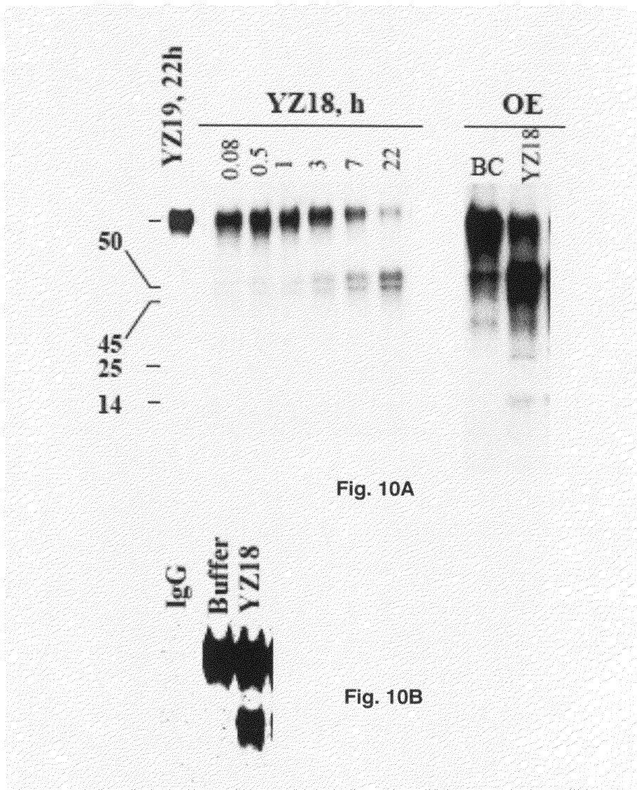

One of the consequences of enhanced nucleophilicity induced by immunization with the E-gp120 is the ability to catalyze the cleavage of gp120. Electrophoresis assays revealed that 3 anti-E-gp120 IgGs cleave gp120 (clones YZ18, YZ20 and YZ24) (FIG. 10 shows an example experiment using IgG YZ18).

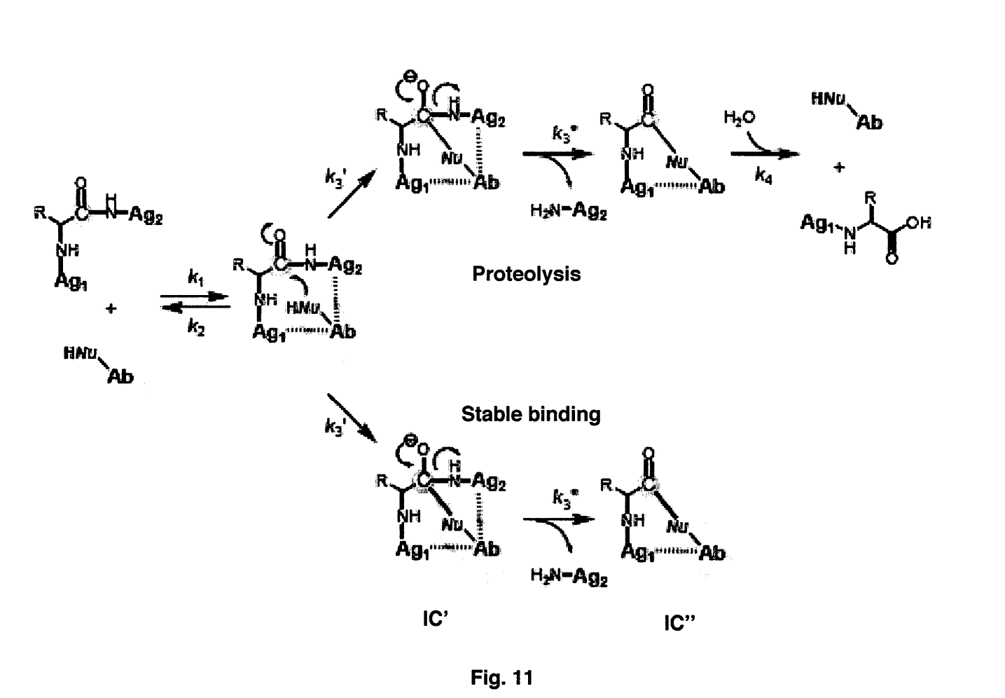

The nucleophilicity of the MAbs is reminiscent of that displayed by conventional serine proteases, in which intramolecular interactions impart nucleophilic reactivity to the active site serine residue, permitting attack on electron deficient reaction centers in the polypeptide substrate, e.g., the carbonyl group of the peptide bond. Inhibitors of serine proteases inhibit the catalytic activity of the anti-E-gp120 MAbs (30). Site directed mutagenesis (38) and crystallography studies (39) of other catalytic MAbs have revealed nucleophilic sites analogous to the Ser-His-Asp catalytic site of serine proteases. Specificity of the nucleophilic MAbs is assured by noncovalent recognition of peptide epitopes in coordination with nucleophilic attack on the antigen (FIG. 11)

### Materials and Methods

E-gp120 preparations and electrophilic probes. Preparation of E-gp120 1a was by acylation of surface accessible amino groups with the succinimidyl ester of diphenyl suberoylamino(4-amidinophenyl)methanephosphonate as described previously (30). E-gp120 1b was prepared by acylation of gp120 with N-(_-maleimidobutyryloxy)succinimide ester, followed by nucleophilic addition of the _-carboxamide of Cys-Glu-Tris (Tris, tris(hydroxymethyl)aminomethane) and diphenyl amino(4-amidinophenyl)methanephosphonate [m/z (ESI) 717.4 (MH⁺; Calcd MH⁺ for C₃₂H₄₁N₆O₉PS, 717.2)]. Monomer/oligomers compositions were determined by densitometry of silver-stained SDS-electrophoresis gels. For trimer enrichment, E-gp120 1a was subjected to gel filtration chromatography on a Superose 6 column (GE Healthcare; 10 mM HEPES-0.15 M NaCl, pH 6.5, containing 0.1 mM CHAPS). The effluent was collected in 0.3 mL fractions and the fractions corresponding to the retention volume of 11.3-12.2 mL were combined. Preparation of E-421-433 was described previously (28), and the control probe containing the shuffle amino acid sequence of 421-433 (E-S421-433) was prepared essentially in the same manner. E-293-311 was prepared from the biotinylated 293-311 (biotinamidohexanoyl-LNESVQINC′TRPNYNKRKR; C′, S-acetamidomethyl-Cys; Genemed Synthesis) by peracylation with the succinimidyl ester of diphenyl suberoylamino(4-amidinophenyl)methanephosphonate (27) followed by HPLC purification [m/z by ESI-MS, 1298.6 (3+; calcd, 1299.8), 974.8 (4+; calcd, 975.1), 779.6 (5+; 780.3)]. Preparation of E-VIP and E-hapten was described previously (27, 40). MAbs. MAbs were prepared from mice immunized with E-gp120 1a or 1b. Adjuvant was monophosphoryl lipid A-trehalose dicorynomycolate emulsion (Ribi adjuvant; Sigma). Hybridomas were prepared by fusion of splenocytes with myeloma cell line (NS-1; Ref: (41)). Screening of IgGs in culture supernatants by a covalent ELISA protocol, in which Abs bound noncovalently to immobilized immunogen (40-100 ng/well) were removed by washing with 2% SDS prior to detection of immune complexes (30), identified gp120 specific nucleophilic MAbs including the following 17 IgG MAbs: YZ18 (1a), YZ19 (1a), YZ20 (1a), YZ21 (1a), YZ22 (1a), YZ23 (1a), YZ24 (1a), SK-T01 (1a), SK-T02 (1a), SK-T03 (1a), SK-T04 (1a), 6B11 (1b), 2F2 (1b), 7H3 (1b), 1F4 (1b), 3A5 (1b), 5E11 (1b). IgG was purified from tissue culture supernatants containing MAbs by affinity chromatography on immobilized protein G (42). Control MAbs (anti-yellow fever virus antigen clone CRL 1689; ATCC) were purified in the same manner.

MAb structure. YZ23 Fab crystals were grown at room temperature by vapor diffusion in hanging drops. Two _L of the Fab stock solution (4 mg/mL in 10 mM Tris, pH 7.5) was mixed with 2 _L of the reservoir solution (16% w/v polyethylene glycol 6000, 0.1 M HEPES, pH 7.4). Rod-shaped crystals appeared belonged to the space group P2₁2₁2₁, with unit cell parameters a=52.29 Å, b=62.92 Å, c=135.17 Å and one Fab per asymmetric unit. The crystals were cryo-protected in 25% glycerol and flash-frozen in liquid nitrogen. Diffraction data to 2.5 Å resolution were collected at 100 K at the Advanced Light Source, beamline 4.2.2, Berkeley. The structure was solved by molecular replacement and refined to the crystallographic R factor of 0.223 using 95% of the data between resolution limits 20-2.5 Å. The free R factor was 0.277 for the remaining 5% of randomly excluded reflections. The final model consisted of 430 amino acid residues and 70 water molecules. Average B-factor was 31.4 Å² for the protein and 27.4 Å² for water molecules. The deviations from standard bond lengths and bond angles were 0.0054 Å and 1.22°, respectively. Ramachandran plot showed 89.3% of amino acid residues in the most favored regions.
HIV neutralization. Infection of peripheral blood mononuclear cells by primary isolates of HIV was measured (26) by treating the virus (100 TCID₅₀; TCID₅₀, 50% tissue culture infectious dose) with an equal volume of increasing concentrations of protein G-purified MAbs for 1 h. Phytohemagglutinin-stimulated cells from healthy human donors were added and cultures incubated for 3 days, the cells were washed, incubated in fresh RPMI for 24 h, lysed with Triton X-100, and p24 in the supernatants was measured. Concentrations yielding 50% and 80% neutralization (IC₅₀ and IC₈₀) were determined from least square fits to the equation, % HIV neutralization=100%/[1+10^{((log IC50-Ab concentration)×Hillslope)}]
Epitope identification. In peptide competition assays, MAbs were allowed to bind E-gp120 (MAb YZ18) or gp120 (other MAbs) coated plates (40 ng/well) in the presence or absence of 15-mer gp120 fragment peptides (50 _g/mL; NIH AIDS Research and Reference Reagent Program) at 37° C. for 2 h in DB8 containing 0.05% Tween20 and 0.25% BSA, and bound MAbs measured by goat anti-mouse IgG-HRP conjugate (1:1000 dilution; Fc specific) and OPD as a substrate (490 nm). To compare epitope specificities, the reference MAbs (IgGs b12, 447-52D, 2G12, 17b, 47d) were allowed to bind gp120 immobilized using sheep Ab directed to gp120 residues 497-511 (Cliniqa) in the presence or absence of anti-E-gp120 MAbs, and the bound reference MAbs were detected with HRP-conjugated goat anti-human IgG (1:1000). The SDS-electrophoresis assay to identifying binary epitope specific MAbs was conducted using two peptide probes E-421-433 and E-293-311, and two control probes E-S421-433 and E-VIP. MAbs (75 _g/mL) incubated with E-peptides (10 _M) for 3 h were subjected to SDS-electrophoresis and MAb-peptide adducts were detected by streptavidin-HRP staining of the blots. Band intensities were determined by densitometry and expressed in arbitrary volume units (AVUs). To analyze expression of various epitopes by E-gp120, MAbs (b12, 45 _g/mL; 447-52D, 0.8 _g/mL; 2G12, 7.5 _g/mL) were incubated with HIV (MN, 1.6×10³ TCID₅₀/mL) in the presence or absence of E-gp120 1a (32 phosphonate groups/gp120) or control gp120, EGF or E-EGF (0.5 _M) for 20 h at 4° C. MAb-bound virions were captured in protein G-coated wells (1 _g/well; 1 h), lysed with 10% Triton X100 (15 min), and HIV p24 in the lysates (1:3 dilution in PBS) were measured by Beckman-Coulter HIV p24 antigen EIA kit. E-gp120 binding by reference MAbs were studied by ELISA in which plates were coated with E-gp120 1a, trimer enriched preparation of 1a, or control gp120 (40 ng/well) and bound MAbs detected by anti-human IgG-HRP (Fc specific; 1:1000).

### EXAMPLE 2

### Dual Epitope Polypeptide Constructs

The minimum structure of dual epitope immunogens is expressed as (Ag)-Li-(SAg), where Ag, SAg and Li represent, respectively, a conventional antigenic determinant, a superantigenic epitope and a linker connecting two epitope components. The linker length and constitution should be such that the SAg and Ag components assume conformations similar or identical to the corresponding regions of the native superantigenic polypeptide. The dual immunogens may contain additional elements, such as carrier proteins to increase immunogenicity, electrophilic groups to promote the synthesis of nucleophilic antibodies (Abs), and accessory groups to help the SAg and Ag assume the desired conformation.

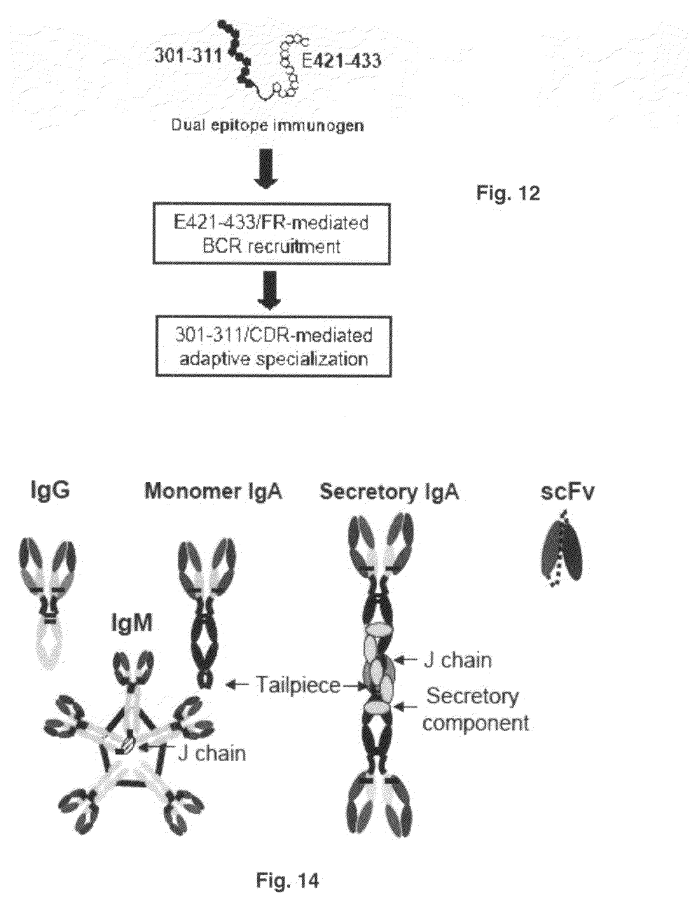

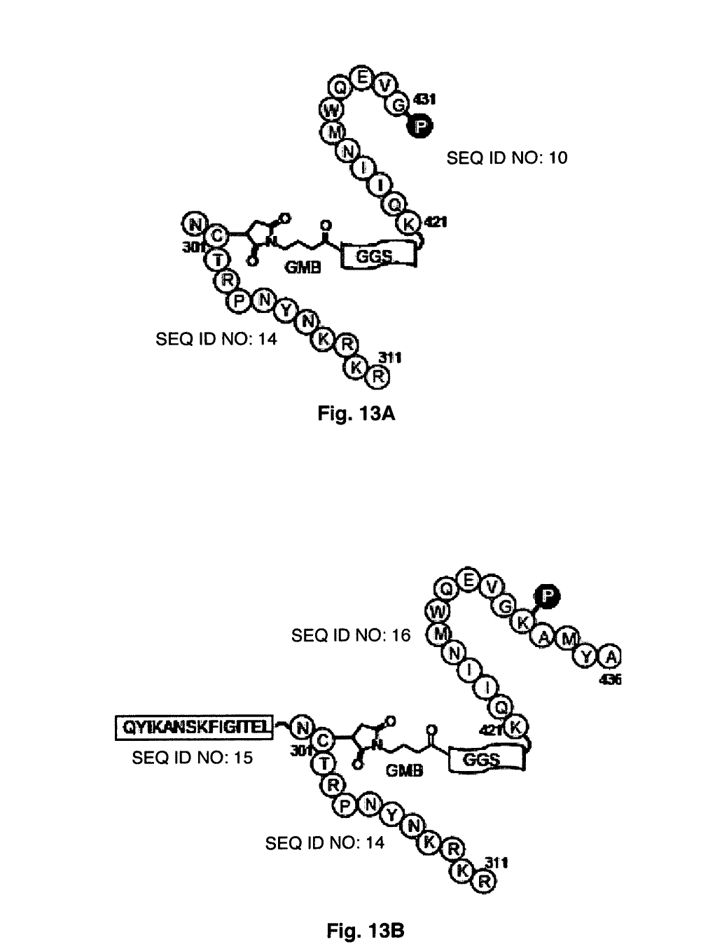

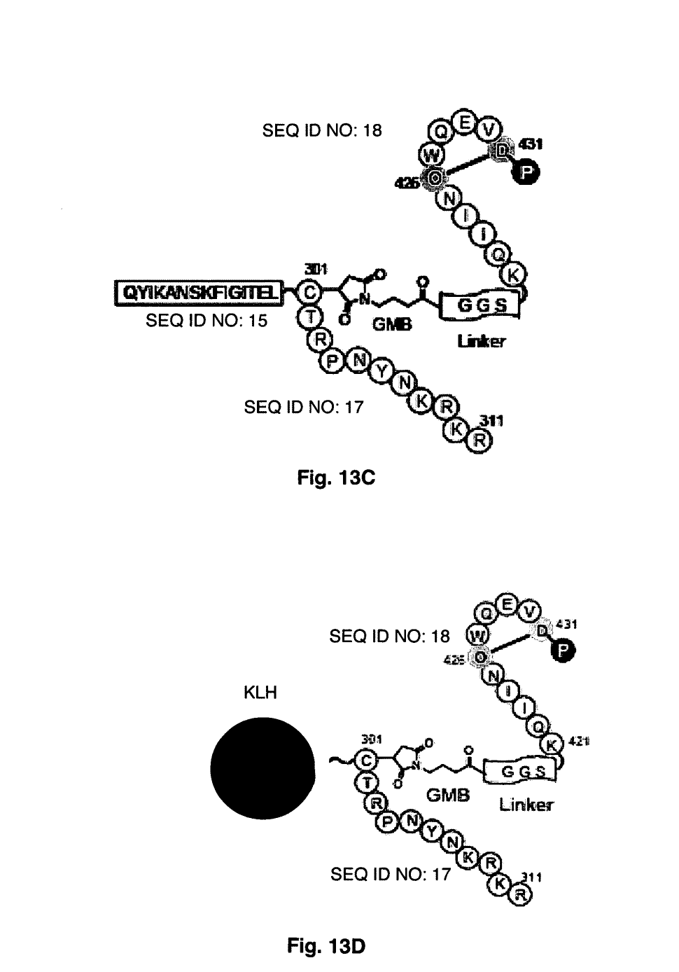

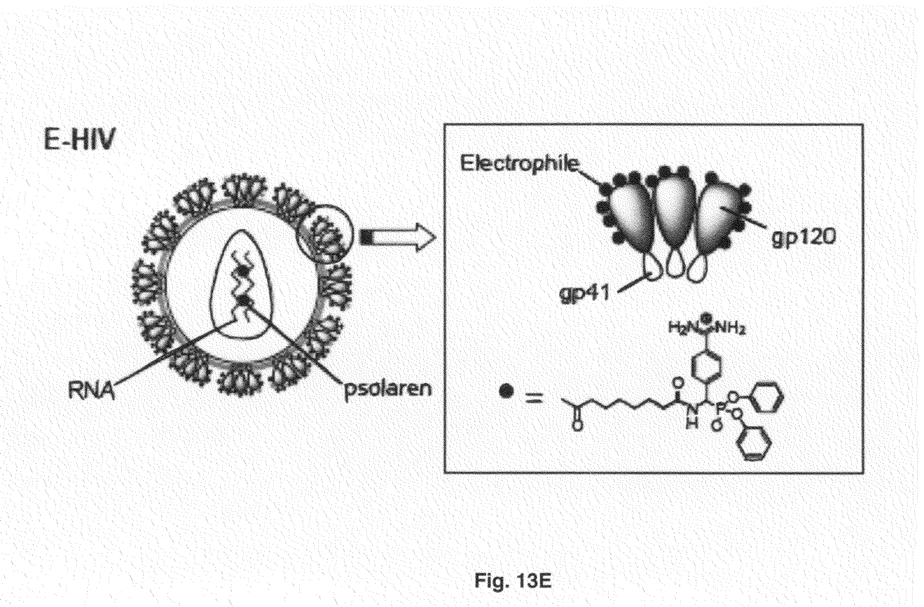

We conceive that a dual epitope immunogen containing the SAg epitope that interacts with BCR framework regions (FRs) and another conventional antigenic epitope that interacts with the complementarity determining regions (CDRs) will drive B cell differentiation into a favorable pathway resulting in improved synthesis of protective Abs (FIG. 12). The hypothesis is based on our findings that neutralizing MAbs to full-length E-gp120 frequently display dual recognition of peptide composed of gp120 residues 421-433 and residues 301-311. Examples of dual epitope constructs composed of these two epitopes are shown in FIG. 13.

The SAg and Ag epitopes need not be limited to the 421-433 and 301-311 regions described in Example I. In principle, any SAg epitope and any conventional epitope are suitable for construction of the dual epitope constructs provided they are sufficiently immunogenic.

In addition to the synthetic peptide constructs described above, purified full-length proteins and the proteins expressed on the surface of viral or cell membranes can be used to induce the synthesis of binary epitope specific Abs. Production of the desired Abs is feasible if the two epitopes, the Ag and SAg, are recognized in concert by CDRs and FRs, respectively.

Example dual epitope constructs: One example of such constructs consists of the covalent gp120 oligomers described in Example I. Additional examples of dual epitope polypeptide constructs are described as follows using HIV gp120 as the example target of the binary epitope specific Abs.

(a) (301-311)-GMB-GGS-(E-421-433): This dual epitope construct is composed of gp120 residues 301-311 as Ag unit, 421-433 as SAg unit and the linker GMB-GGS (_-maleimidobutyryl-Gly-Gly-Ser) (FIG. 13A). The SAg component contains the electrophilic group at the C-terminus for stimulation of the synthesis of nucleophilic antibodies.

T-(301-311)-GMB-GGS-(E415-436): This example contains a defined T epitope at the N terminus (T, 15 residues tetanus toxoid peptide QYIKANSKFIGITEL; ref (1)) as a “universal” T epitope (FIG. 13B). The SAg component is E-415-436 hypothesized to assume the neutralization relevant conformation of 421-433. From NMR and CD studies (24), residues 416-419 have been identified as a switch region that induces a helical conformation in the 421-433 region with improved CD4 binding activity. Peptide termini are usually more flexible than internal peptide components, and inclusion of the additional 434-436 residues at the C terminus should impart greater rigidity to the targeted 421-433 region. The electrophilic group is placed on the side chain of Lys432. This group enjoys spatial freedom because of linker flexibility, allowing it to pair with nucleophiles that can potentially be located at variable positions within the BCR antigen binding site.

(b) T-(301-311)-GMB-GGS-(E-c421-433): This construct contains a conformationally constrained analog of gp120 residues 421-433 (FIG. 13C). The purpose of constraining the 421-433 region is to reduce flexibility and approximate a potential _-sheet conformation. Cyclization constraints have often proven successful in the case of _-sheet peptide regions. _sheet formation in this gp120 region and a turn at residues 427-430 are evident in the published crystal structure of monomer gp120 (5), and the distance between the _-carbons of Met425 and Gly431 is comparable to the distance between the _-carbons of the side chain-linked dipeptide Orn-Asp (Orn, ornithine). Such a structure may be simulated by replacing Met425/Gly431 with Orn/Asp residues to cyclize the peptide.

(c) KLH-(301-311)-GMB-GGS-(E-c421-433): This example contains the carrier protein Keyhole Limpet Hemocyanin (KLH) (FIG. 13D). KLH contains multiple T cell epitopes and is a commonly used carriers for antibody production in the conjugation with poorly immunogenic peptides.

(d) E-gp120 oligomers and E-HIV: Covalently stabilized E-gp120 expressing the 301-311 and 421-433 epitopes in sufficiently immunogenic form are described in Example I. E-HIV is an analogous preparation consisting of whole virus particles in which the coat proteins are modified to display electrophilic phopshonate groups (FIG. 13E). The main advantage of E-HIV is that it contains native, oligomeric gp120. The oligomeric gp120 architecture is conceived to be stabilized by covalent cross-linking between the electrophilic group and natural gp120 nucleophile (6). In addition, gp120-gp41 association may be also stabilized by the same mechanism. E-HIV preparations can readily be inactivated by psoralen to eliminate viral infectivity (7). Psoralen binds viral RNA covalently and it inactivates HIV with minimal protein denaturation. While lab-adapted strains of HIV may be grown to high titer in continuous cell lines, it has been shown that glycoprotein spikes are more stable and at higher density on primary isolates. A primary clade C virus (strain 97ZA012) can be grown to high titers in a continuous human T-cell line (PM-1) highly permissive to primary HIV isolates. By producing the virus in Cellmax continuous flow (hollow-fiber) perfusion bioreactors, infectious titers of up to 6.25 log/ml and gp120 yields of approximately 900 ng/ml are routinely obtained. The harvested viruses can be further purified by pelleting through sucrose without significant loss of env.

Electrophilic group: The electrophilic groups can be located in one of the two epitopes of the dual epitope constructs or in epitopes (Ag and SAg epitopes). The examples in FIG. 13 contain the phosphonate diphenyl ester group as an electrophilic component at the C-terminus or the Lys side chain of the SAg component. Other electrophilic groups suitable for induction of binary epitope specific Abs with enhanced nucleophilic reactivity include mono- and di-esters of phosphonic acids and various aryl and alkyl alcohols, _-keto acids, and haloalkyl ketones. The electrophilic group can also contain an additional functionality that captures a water molecule in vicinity of the electrophilic center.
Linker: The example synthetic peptide structures shown here contain the GMB-GGS linker (GMB, _-maleimidobutyryl; GGS, Gly-Gly-Ser). Other linkers can be conceived to optimize the structure of the dual epitope constructs so that the SAg and Ag combinations are present in the correct spatial relationship to each other. Screening of the linker structure can be done by molecular modeling or by empirical means to identify the best linker. The length of fully extended GMB-GGS linker is 21.3 Å. In the published monomer gp120 crystal structure (5), the 301-311 N-terminus is located 14.8 Å from the 421-433 N-terminus. Deletion of each amino acid residue will decrease linker length by 3.7 Å. Thus, linkers with length 17.6, 13.9 and 10.2 Å can be prepared by deleting 1, 2 and 3 residues, respectively, producing dual-epitope constructs with varying inter-terminus distances. Similarly, addition of an amino acid residue will increase linker length by 3.7 Å, and constructs with inter-epitope distances of 25.0, 28.7 and 32.4 Å can be prepared by adding 1, 2 and 3 residues, respectively. A similar design approach was used previously to develop linkers for joining the V_{L}/V_{H} domains in single chain Fv constructs (8). The dual epitope constructs containing various linkers can also be docked by molecular modeling into the two binding sites of a binary epitope specific Ab (e.g., the neutralizing MAb YZ23 described in Example I; the structure of this MAb has been determined by the crystallography). This will rule out obvious steric conflicts. The search process consists of rigid body displacements and permitted rotations around single bonds according to the rotamer database. Consensus scoring options are used to identify docking conformations that rank high in more than one scoring functions. Software modules used to arrive at the final structures are LigScore2, PLP, Jain, PMF, and LUDI (Accelrys). Flexible docking software is also available in our lab (LigandFit). The dual epitope constructs devoid of steric conflicts is screened for reactivity with the reference neutralizing Ab (e.g., MAb YZ23). This procedure helps assure that the epitopes are in their ‘neutralization-relevant conformation’. Dual reactivity can be determined by, for example, the following assays: (a) total binding (noncovalent+covalent) by ELISA; and (b) irreversible binding by SDS-gel electrophoresis (see Example I for methods).
Chemical synthesis: Synthesis of the dual-epitope constructs is straightforward. First, the GMB-linker peptide and the E-421-433 region are prepared as a single unit. This unit is linked to the —SH side chain of residue 301 in the 301-311 peptide, yielding the desired immunogen, which can be purified by HPLC to chromatographic homogeneity and characterized by electro-spray mass spectroscopy (and NMR if needed). The Orn425-Asp431 cyclized analog can be synthesized by regioselective cyclization and phosphonate introduction as follows: (a) Peptide assembly on the Wang resin using TFA-resistant protecting groups, except Orn425 (4-methyltrityl, Mtt) and Asp431 (2-phenyl-isopropyl, 2-PhiPr); (b) removal of Orn and Asp protecting groups using 1% trifluoroacetic acid (TFA); (c) on-resin cyclization using the coupling reagent HATU, peptide cleavage with TFA and condensation with the aminophosphonate precursor; and (d) deprotection with 1M trifluoromethanesulfonic acid. Cyclization reactions can be conducted at several alternate neighboring residues to test the resulting constructs for reactivity with our available neutralizing Abs to select the best compound for immunization.

E-HIV preparation is as follows. The virus is inactivated by aminomethyltrimethylpsoralen (10 μg/ml) along with ascorbic acid (0.5 mM) to quench secondary oxidative reactions. The preparation is exposed to 3 Joules (3 min) of ultraviolet light (UVA) [path length=3 mm] in two cycles of treatment. No infectious virus can be detected following this treatment in concentrated preparations in PBMC assays (reduction in infectivity>6 logs). The preparation will be purified further by gel filtration (Superose-6 column) allowing recovery of the virus in the void volume (this helps remove to remove host cell proteins). Derivitization with the phosphonate diester groups is done by incubating the inactivated viruses with the active ester of diphenyl N-suberoyl-amino(4-amidinomethyl)methanephosphonate. Another gel filtration is done to remove free phosphonates, along with any shed gp120 (free gp120 content in the immunogen will be minimized as far as is possible). gp120 content in the final preparation is measured by enzymeimmunoassay. Derivitization with the phosphonate groups is expected to stabilize the envelope, as the phosphonates will render the proteins more reactive with natural electrophiles found in their binding proteins (analogous to phosphonate reaction with Abs).

Murine immunizations: The candidate vaccine constructs are validated by conducting immunization using, for example, BALB/c mice hosts (10/group, 4-6 wk old). An appropriate adjuvant is selected. The adjuvant is critical in determining the class of Abs produced by the B cells, the magnitude of the Ab response, B cell migration patterns and even the specificity of the induced Abs. The adjuvant influences on T-independent and T-dependent B responses via cytokine induction and direct interactions with cellular pattern recognizing receptors, including toll-like receptors (TLRs). For example, to achieve preferential induction of the desired Abs by mucosal immunization, nasal immunizations are conducted with several adjuvants: (a) lipopolysaccharide (LPS; stimulates T-dependent and independent responses, TLR4 stimulant); (b) CpG (favors TH1 response, signals through TLR9); and (c) Anthrax edema toxin (TH2-response, induces vaginal Ab responses, upregulates costimulatory molecules). The optimal route of immunization is also determined empirically, e.g., nasal, vaginal, oral, intramuscular, intraperitoneal and intravenous. In one preferred embodiment of the invention, the nasal route has been shown to effectively stimulate an Ab response to candidate vaccines (see Example III). The dose of the immunogen is varied (10-200 μg peptide equivalents/kg) to determine the optimal dose. Repeat immunizations are conducted to stimulate memory B cell responses, for example, 3-4 immunizations at 2-4 week intervals.

Five days after each immunization, blood and cervicovaginal lavage fluid (CVLF) is collected, Abs present in the sera (IgM, IgG and IgA) and CVLF (sIgA, IgG) are purified to electrophoretic homogeneity by affinity chromatography using immobilized anti-IgM Abs, Protein G and anti-IgA Abs as in our previous studies (6, 9, 10) and studied as follows.

(a) Catalysis and binding. The following substrates are tested: (a) intact virions; and (b) recombinant monomer gp120 (strain MN). Intact virions (strain ZA009, clade C, primary isolate, R5 dependent) and MN (clade B, lab adapted, X4 dependent) are obtained by growth in human PBMC, quantified by infectivity assays and purified by gel filtration. After incubations of the Abs (1-100 nM) with the virion preparations (˜10³ TCID₅₀), hydrolysis of viral gp120 is measured by a method similar to our published monomer gp120 hydrolysis assays, i.e., reducing SDS-electrophoresis and immunoblotting with anti-gp120 Åbs (6). gp120 hydrolysis is evident as depletion of the intact gp120 band and appearance of low mass fragments. If needed, viral gp120 is labeled with ³⁵S-Met for enhanced detection (by infecting PBMCs with HIV in ³⁵S-Met-containing medium). Hydrolysis of unrelated polypeptides is determined by electrophoresis to confirm lack of promiscuous catalytic activity [ref (11); e.g., biotinylated albumin, ovalbumin, calmodulin, Factor VIII]. Hydrolysis rate data is obtained at varying concentrations of the best set of hyperimmune Abs. Apparent Kₘ and Vₘₐₓ are computed as in ref (11).

Ab-virus binding can also result in viral neutralization. Ab binding to monomer gp120 is determined by ELISA (6, 12). For intact virus binding, virions (˜103 TCID₅₀; strains ZA009 and MN) are incubated with varying Ab concentrations to determine the concentrations yielding a detectable reaction. Preimmune Abs, MAb SKT03 (which binds irreversibly to a non-neutralizing epitope) and a reversibly binding MAb (e.g., clone 257DIV) are included as controls. MAb-virion complexes are captured using immobilized Protein G and unbound virions are washed away. p24 is solubilized using Triton X-100 and measured by p24 ELISA. To measure stability, the Abs are reacted with the virions for a suitable length of time and excess recombinant gp120 (I μM) is added to induce dissociation of noncovalently associated complexes. The reaction mixtures are subjected to gel filtration (13, 14). The detergent Empigen is used to disrupt virions without dissociating immune complexes (15). An Ab to the C-terminal region of gp120 is employed to capture the gp120-Ab complexes and the reaction is developed using peroxidase-conjugated anti-mouse L chain Ab. To measure t_, the reaction mixture is incubated for prolonged periods, residual undissociated immune complexes are measured, and t_ obtained by fitting the data to the first order rate equation.

(b) Neutralization. Initially, unpurified serum and CVLF from preimmune and hyperimmune mice are used to determine neutralization of the clade C strain ZA009 (R5-dependent; primary isolate) in PBMC cultures. Repeat assays are done using Abs purified from serum and CVLF (sIgA, IgM, IgA and IgG). Neutralization assays are done as described (16, 17). Virus stocks (100 TCID₅₀/well) and Abs (ng/ml-μg/ml) are incubated for a short period, whereupon the virus is allowed to infect PHA-stimulated PBMCs. Viral infection is quantified by measuring p24 by EIA. The endpoint titer for neutralization is defined as the interpolated titer at which there is 50% or 80% inhibition of p24 expression relative to controls incubated without Ab. Controls include equivalently purified Abs from nonimmune mice. Nonspecific cytotoxicity is analyzed using PBMCs treated with Abs without virus by microscopy and vital staining of the cells. Breadth of neutralization is studied using a panel of strains that include R5, X4 and R5X4 strains. Residues 421-433, are comparatively conserved but some strains show divergences. Strains with sequence differences at defined 421-433 positions are also included: e.g., strains 98BR004, 98IN022 and 97ZA012. Four to 6 strains drawn from clades A, B, C, D, E (EA env recombination) are studied to establish neutralization of diverse HIV strains.

Non-human primate validation: Unlike other experimental animals, monkeys are susceptible to infection by SIV strains expressing the HIV proteins, e.g., SHIV_{SF162P3} contains the env, tat, rev and vpu sequences from the CCR5-tropic (R5) primary HIV strain SF162 (clade B) cloned into the SIV_{mac239} genome. Vaccine candidates can readily be tested in rhesus macaques using such model strains. Detailed descriptions of the methods are available (e.g., refs (18-22). Briefly, groups of rhesus macaques are immunized repeatedly via the appropriate route with the candidate vaccine in combination with the appropriate adjuvant at various time intervals. After induction of HIV neutralizing Abs in serum and CVLF has been confirmed, the macaques are challenged vaginally with an infective dose of SHIV_{SF162P3}. SHIV infection is monitored over 5-9 months in blood and CVLF by routine RT-PCR methods. At necropsy, the presence of HIV reservoirs in various tissues is monitored. Mean viral loads and CD4 cell counts in immunized and control animals are computed and statistical significance of the differences is estimated using appropriate tests. The monkeys are also examined for signs of clinical disease, including monitoring for posture, mobility, food consumption, behavior, body weight and vaginal or systemic distress. Blood samples are analyzed by CBC, hematocrit, serum chemistry, creatinine, alkaline phosphatase and aspartic serum transferase tests. At euthanasia, a complete necropsy will be done, including histopathology of lymphoid tissues.

### EXAMPLE 3

### Naturally Occurring Proteolytic Antibodies to the Gp120 Superantigenic Site and Their Inducibility

An effective vaccine to HIV must induce robust polyclonal antibody (Ab) responses to one or more conserved epitope that neutralizes viral infecting. While some monoclonal (MAbs) to comparatively conserved HIV epitopes are known, there are no reports of immunogens that induce Abs with sufficiently robust HIV neutralizing activity attributable to recognition of a conserved HIV epitope and detectable in polyclonal Abs found at the sites of HIV infection. We tested the ability of an electrophilic analog of gp120 residues 421-433 (E-421-433) to induce catalytic and neutralizing polyclonal Ab responses in mice. As noted previously, residues 421-433 express superantigenic character, a property that may limit the adaptive B cell response to this region of gp120. Moreover, synthetic peptides can assume various conformations that may diverge from the native conformation of the peptide in the full-length protein, with the result that the induced Abs may not recognize the full-length protein. Unexpectedly, we observed favorable response to this immunogen in IgM class Abs and secretory IgA (sIgA) class Abs that can be conceived as the basis of an effective HIV vaccine.

gp120 hydrolyzing Abs in humans without HIV infection: Our studies on proteolytic Abs in humans without HIV infection have yielded unexpected information that is valuable for designing an HIV vaccine. The Ab repertoire is generated from constant (_, _, _, _, _) and V domain genes (V, D, J genes) (FIG. 14). Promiscuous Ab proteolytic activity is a heritable trait, suggested by studies on a germline V domain (1). Consequently, under favorable selection pressures, emergence of antigen-specific proteolytic Abs is feasible. gp120-cleaving IgM class Abs are found in the preimmune repertoire of humans and mice that have not been exposed to HIV (2). The Abs could express enhanced or diminished proteolytic activity over the course of B cell maturation attendant to CDR or FR sequence diversification or class switching recombination from IgM to IgG or IgA producing B cells.

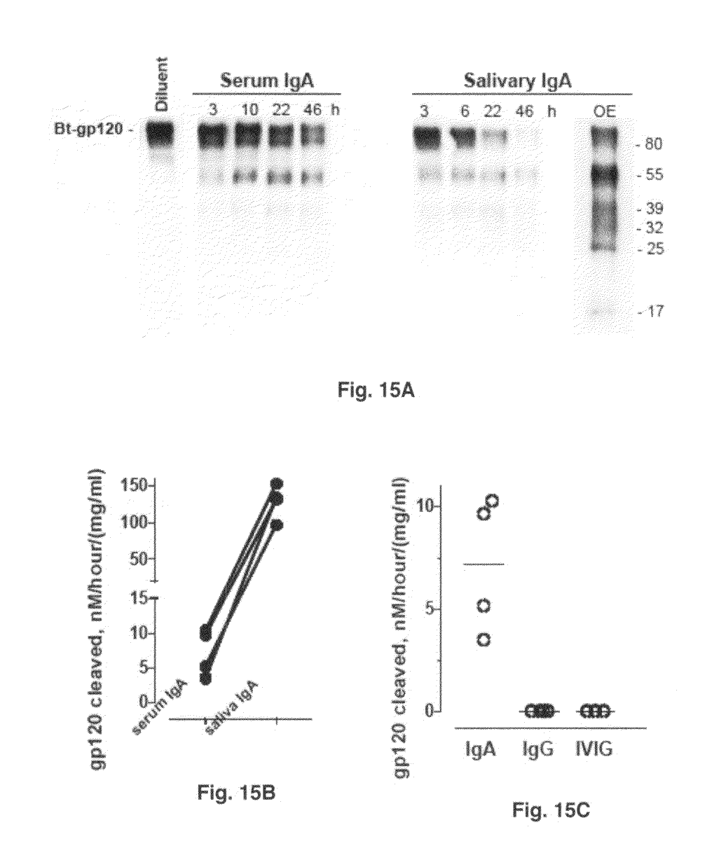

We observed recently (3) that each IgA preparation purified from the saliva and serum of 4 humans without HIV infection cleaved biotinylated-gp120 (Bt-gp120), assessed by depletion of the parent gp120 band and appearance of lower mass fragments in electrophoresis gels (FIG. 15A). The Bt-gp120 product profiles observed using secretory IgA (sIgA) from saliva and IgA from serum as catalysts were essentially identical (products with nominal mass 80, 55, 39, 32, 25 and 17 kD). The number of combining sites per Ab molecule can vary (2 sites/monomer IgA and IgG; 4 sites/salivary IgA dimer). However, the number of combining sites per unit mass of the monomers and aggregates is comparable (one combining site per 75-100 kD), and normalization of the activity data to constant concentration (mg/ml) permits valid comparison of their activities. The mean proteolytic activity of sIgA from the 4 subjects was 15.4-fold greater than serum IgA [means±s.d. respectively, 128.6±23.3 and 7.2±3.3 nM gp120/hour/(mg IgA/ml); FIG. 15B]. The sIgA and serum IgA concentrations yielding detectable catalytic activity are about 2 orders of magnitude lower than the physiological concentrations of IgA in serum (1.5-2.6 mg/ml)(4) and saliva (110-300 μg/ml) (5, 6). Serum IgG fractions were devoid of detectable activity (FIG. 15C. Essentially identical results were obtained using serum IgA and IgG purified from the pooled sera of 34 HIV-seronegative humans (941 nM gp120 cleaved/h/mg IgA; undetectable gp120 cleavage at equivalent IgG concentration.

Reducing SDS-electrophoresis of serum IgA obtained by affinity chromatography revealed the heavy chain (60 kD) and light chain (25 kD) subunits. Salivary IgA contained these bands along with the additional band stainable with anti-secretory component Ab (85 kD). To test for contaminating conventional proteases, serum and salivary IgA preparations purified by affinity chromatography using the anti-IgA column were subjected to further FPLC-gel filtration in a denaturing solvent (6 M guanidine hydrochloride). IgAs can form noncovalent and S—S bonded multimers (7). As reported previously, peaks corresponding to polymeric, dimeric and monomeric IgA were evident (nominal mass, respectively, 915 kD, 433 kD and 153 kD) (8). Each of these IgA fractions displayed reducing SDS-gel electrophoresis profiles identical to those of the starting affinity-purified IgA loaded on the column. Monomer IgA was the predominant serum IgA species recovered from the column (82%; Rₜ 55.2 min). Ninety percent of the salivary IgA eluted as dimers (Rₜ 42.7 min) and higher order aggregates (Rₜ 33.7 min). Following refolding by removal of guanidine hydrochloride, the monomer serum IgA species recovered from the column displayed gp120 cleaving activity identical in magnitude to the affinity-purified IgA preparation loaded on the column (respectively, 630±167 and 823 nM±130 gp120/h/mg IgA), fulfilling the criterion of purification to constant specific activity. The refolded dimeric and higher order salivary IgA aggregates eluting from the column also displayed gp120 cleaving activity, confirming that the predominant form of secretory IgA is catalytically active.

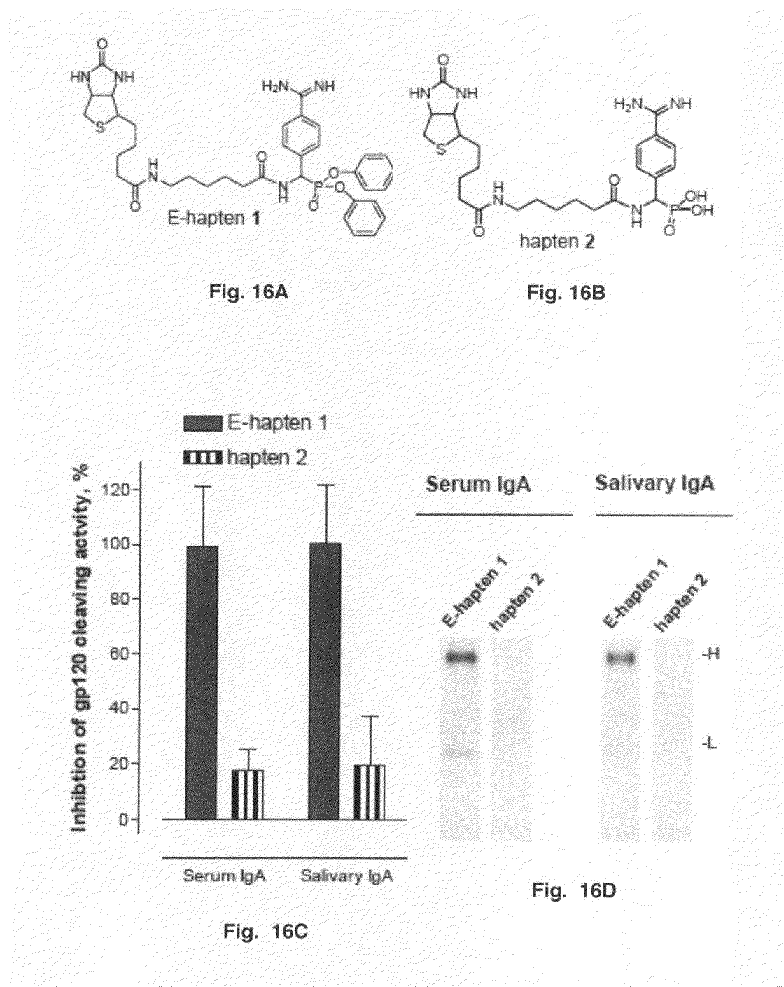

An electrophilic phosphonate diester (E-hapten 1, FIG. 16A) known to bind nucleophilic sites covalently (9) inhibited the catalytic activity of salivary and serum IgA (FIG. 16B). Class-selective inhibitors of metalloproteases (EDTA, 2 mM) and 1,10 phenanthroline (1 mM), cysteine proteases (iodoacetamide, 100 μM) and acid proteases (pepstatin A, 1 μM) did not detectably inhibit the hydrolysis of gp120 by the pooled serum and salivary IgA. The salivary and serum IgA preparations formed adducts with E-hapten 1 stable to heating (100° C., 5 min) and denaturation with SDS, corresponding to the dominant 60 kD heavy chain adduct band and the weaker 25 kD light chain adduct band shown in FIG. 16C.

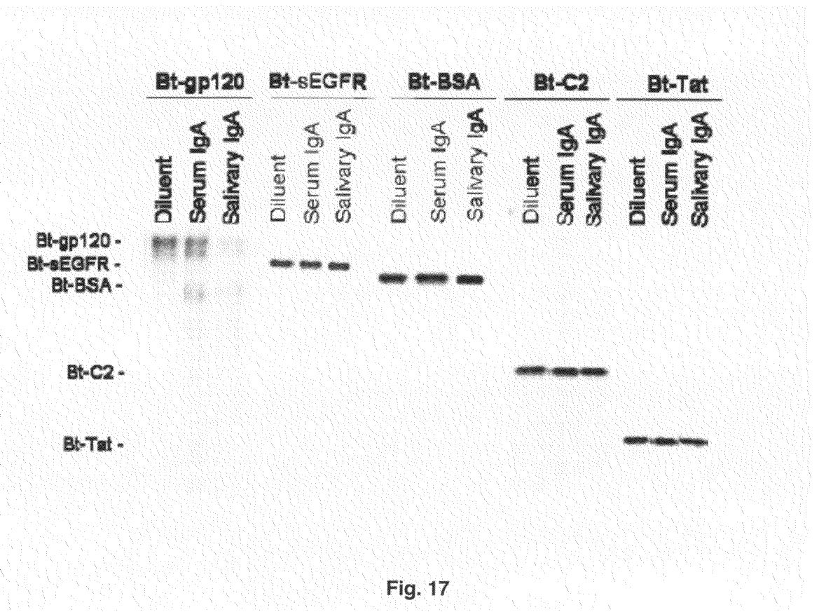

Treatment of Bt-BSA, Bt-FVIII C2 domain, Bt-Tat or Bt-sEGFR with human salivary IgA or serum IgA did not result in noticeable depletion of electrophoresis bands corresponding to the full-length form of these proteins (FIG. 17). Under these conditions, readily detectable Bt-gp120 cleavage was observed, indicating that the catalytic reaction is selective for gp120.

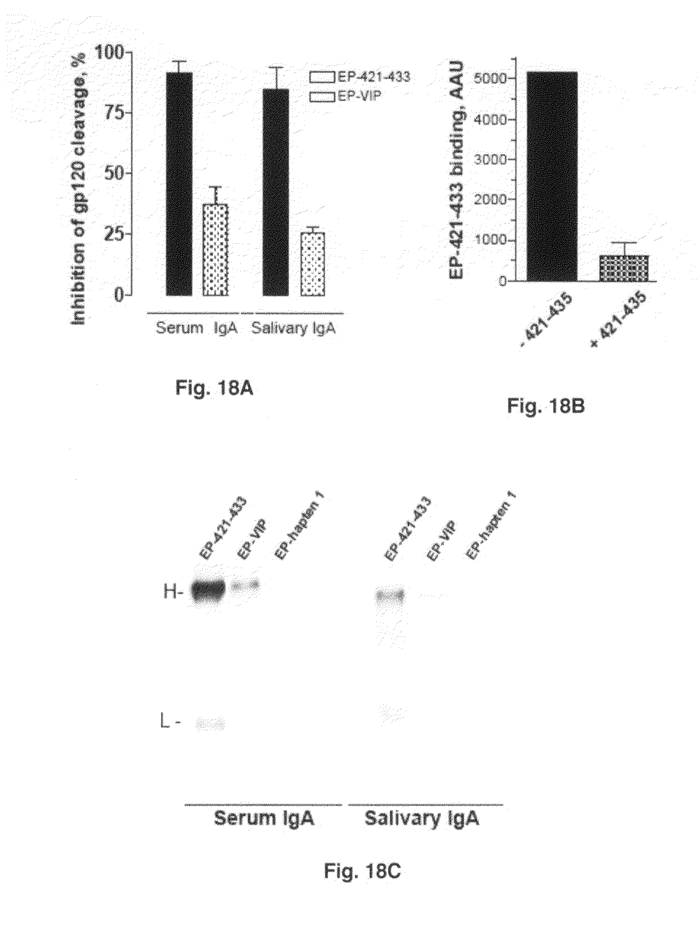

Synthetic peptides containing gp120 residues 421-433 are reported to inhibit noncovalent Ab binding to gp120_{SAg} competitively (10, 11). Increasing concentrations of E-421-433 (10-100 μM), the electrophilic analog of gp120 residues 421-433 containing the phosphonate diester and biotin groups, progressively inhibited the cleavage of Bt-gp120 by salivary IgA (by 21-85%) and serum IgA (by 41-91%). The inhibitory effect of E-421-433 was stronger than the irrelevant control probe E-VIP (FIG. 18A). Similarly, E-421-433 displayed superior irreversible binding to the IgAs compared to control E-VIP, estimated by electrophoretic estimation of the adducts (FIG. 18B). Inclusion of the gp120 peptide 421-435 devoid of the phosphonate group in the reaction mixtures inhibited the formation of the IgA:E-421-433 adducts (FIG. 18C). These observations suggest a nucleophilic mechanism of IgA catalysis in which noncovalent recognition of the 421-433 peptide region contributes to the observed selectivity for gp120.

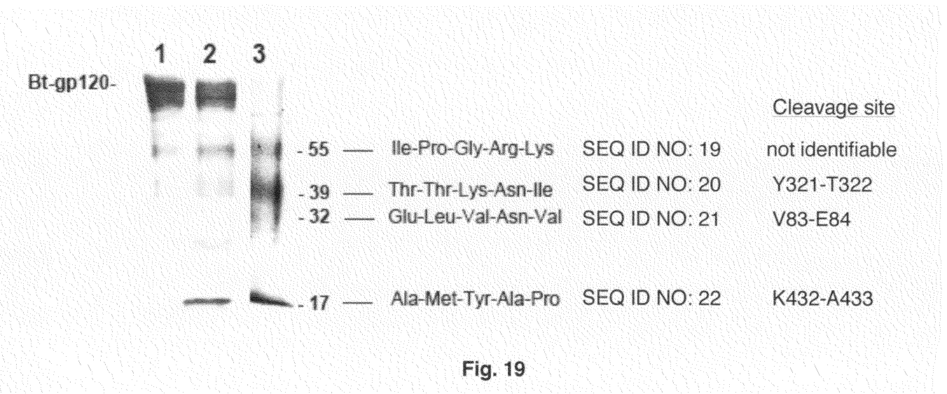

To identify the cleavage sites, gp120 was digested by polyclonal salivary IgA and the gp120 fragments obtained by SDS-electrophoresis were subjected to N-terminal amino acid sequencing. Product bands at 55, 39 and 17 kD and a faint band at 32 kD were evident (FIG. 19). The 55 kD fragment yielded a sequence corresponding to the N-terminus of gp120. The remaining fragments yielded N terminal sequences corresponding to gp120 residues 84-88, 322-326 and 433-437, indicating cleavage at the following peptide bonds: Val83-Glu-84 (located in the gp120 C1 domain), Tyr321-Thr322 (V3 domain) and Lys432-Ala433 (C4 domain).

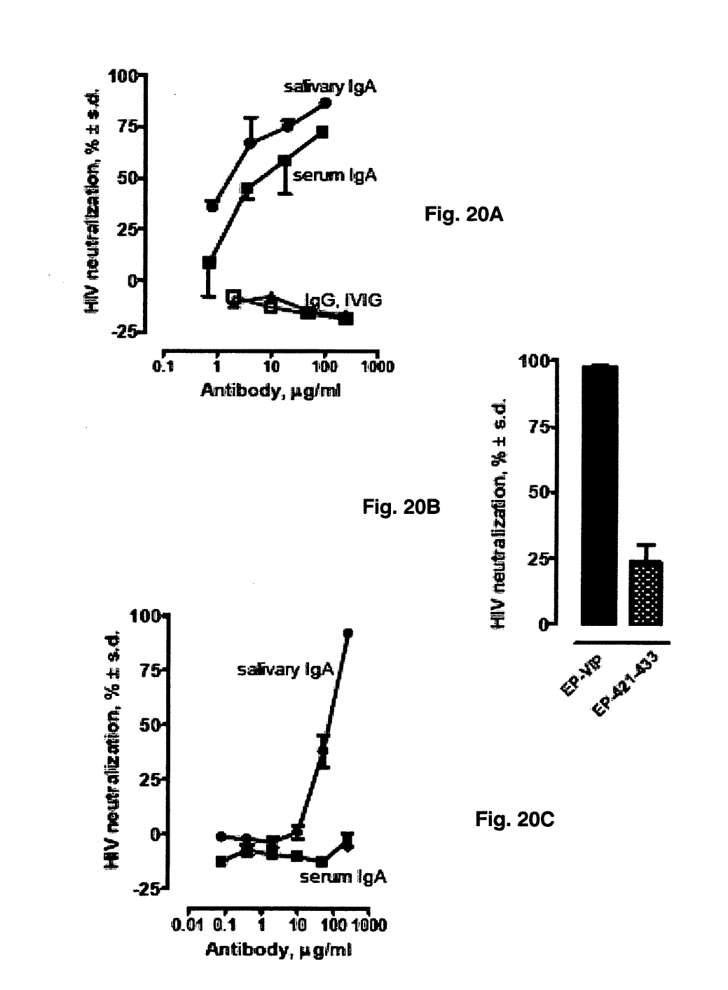

We studied the effect of the Abs on infection of human PBMCs by the HIV-1 strain 97ZA009 (clade C, chemokine coreceptor R5 dependent). The sequence of gp120 residues 421-433 in this virus strain and the recombinant gp120 employed in catalysis studies is identical except for a conservative Arg/Lys substitution (KQIINMWQEVGR/KA). Pooled serum IgA and salivary IgA from uninfected donors displayed reproducible and dose-dependent neutralizing activity, respectively, in each of 5 and 8 assays conducted with these antibody preparations (mean half maximal effective concentration (EC₅₀) s.e.m, serum IgA 62.0±38.3 μg/ml; salivary IgA 48.5±14.5 μg/ml). FIG. 20A reports the neutralizing activity of serum IgA and salivary IgA studied in parallel (identical virus preparation, identical host PBMC preparation). Unlike the IgAs, serum IgG prepared in our lab and 3 commercial IgG preparations (IVIGs; Gammagard S/D, Inveegam EN and Intratect) did not display detectable neutralizing activity. As expected, the neutralizing activity was maintained by treatment of the IgA with the irrelevant probe E-VIP, but inclusion of E-421-433 in the IgA-virus mixture resulted in inhibition of the neutralizing activity (FIG. 20B). Viral neutralization by salivary IgA was reproducibly observed following comparatively short (1 h) incubation with HIV, whereas neutralization by serum IgA was evident only upon prolonged IgA-virus incubations (24 h; FIG. 20C). A second primary HIV isolate, 92BR020 (clade B, R5-dependent) was also neutralized by the salivary IgA preparation employed in FIG. 20C (EC₅₀, 5.0±2.1 μg/ml).

From these studies, it is evident that IgA class Abs (and the previously published IgM class Abs, ref (2)) but not IgG class Abs can hydrolyze gp120 and neutralize HIV by recognition of gp120 the superantigenic site. The following considerations are relevant to interpreting these observations. First, BCR-antigen engagement is thought to drive B cell division. BCR-catalyzed gp120 cleavage will result in release of antigen fragments, depriving the cells of the proliferative signal. Therefore, the catalytic activity can improve adaptively only to the extent that product release occurs more slowly than transmembrane signaling responsible for stimulating cell division. In this scenario, the superior proteolytic activity of IgMs/IgAs may be due to more rapid transmembrane signaling by μ/_-BCRs compared to _-BCRs. Second, BCR catalysis may be a proliferative signal itself. Peptide bond cleavage liberates a large amount of energy (˜_{—}70 kcal/mole) compared to far smaller energies available from noncovalent BCR-antigen engagement. It is conceivable that some of the energy is employed to induce a productive BCR conformation transition needed for signal transduction or increase the rate of BCR diffusion in the lipid bilayer, thus increasing the probability of BCR cross-linking. Third, BCR-catalyzed gp120 cleavage may mitigate the B cell apoptotic effect resulting from interaction of the gp120 superantigenic site at the BCR FRs (by aborting apoptotic signal transduction due to release of the antigen fragments). Readily detectable catalytic cleavage of gp120 by a VH2+ Ab that did not bind gp120 appreciably was evident in our hands (2), and increased synthesis of certain non-VH3 families has been described following HIV infection (12). From this reasoning, it can be hypothesized that synthesis of proteolytic Abs specific for the gp120 superantigenic site may be less restricted than of Abs that only bind the site.

Abs induced by E-421-433 immunization: Previously, we reported that a keyhole limpet hemocyanin (KLH) conjugate of peptide 421-436 devoid of the electrophile induced Abs that recognized full-length gp120 (13). Here, groups of mice were immunization with the KLH conjugated E-421-433 in the adjuvant LTm or Cholera toxin subunit B/IL12 by the nasal route. LTm is a R192G mutant of heat labile E. coli enterotoxin known to stimulate vigorous mucosal Ab responses (14); provided by Dr. Clements). The mutation renders the protein non-toxic by knocking out the ability to interact with Gs_-mediated signal transduction. 1725 moles E-421-433 were present per mole of KLH in the immunogen. The following assays were conducted using sera, saliva or vaginal fluid collected from the immunized mice: (a) E-421-433 binding by ELISA; (b) gp120 hydrolysis by electrophoresis; and (c) HIV strain ZA009 neutralization (PBMC host). Importantly, these assays may reflect the activity of different Ab subpopulations. The binding assay detects peptide analog binders regardless of neutralizing activity, and it may not detect catalysts or binders specific to viral (trimeric) gp120. The catalysis assay detects catalysts that hydrolyze monomer gp120 regardless of neutralizing activity, it does not detect binders, and it may not detect catalysts specific for viral gp120. The neutralization assay detects only binders and catalysts that recognize viral gp120 and interfere with viral infection.

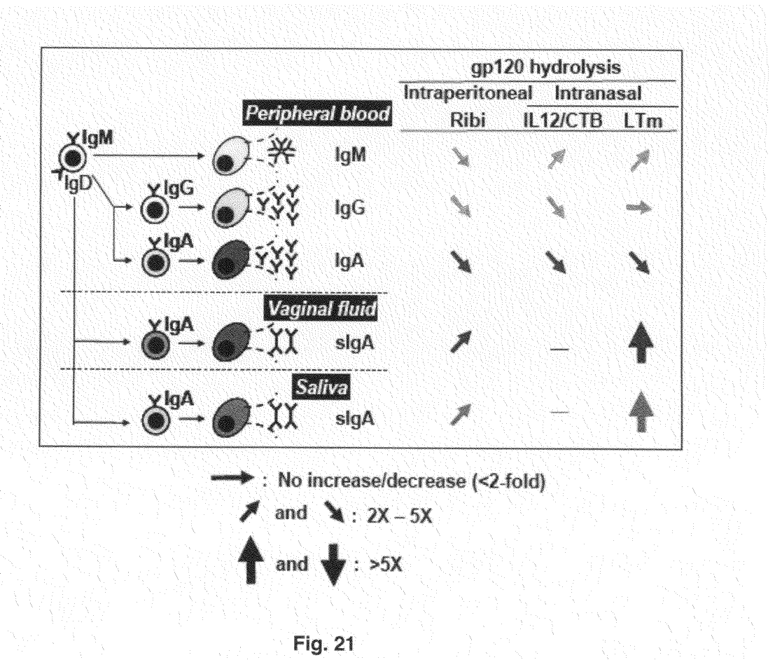

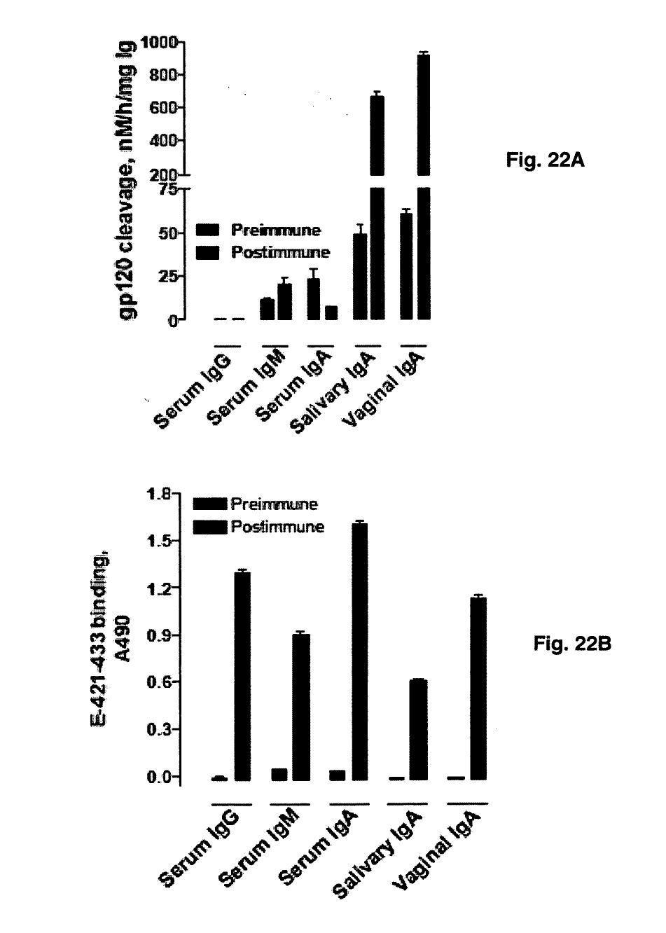

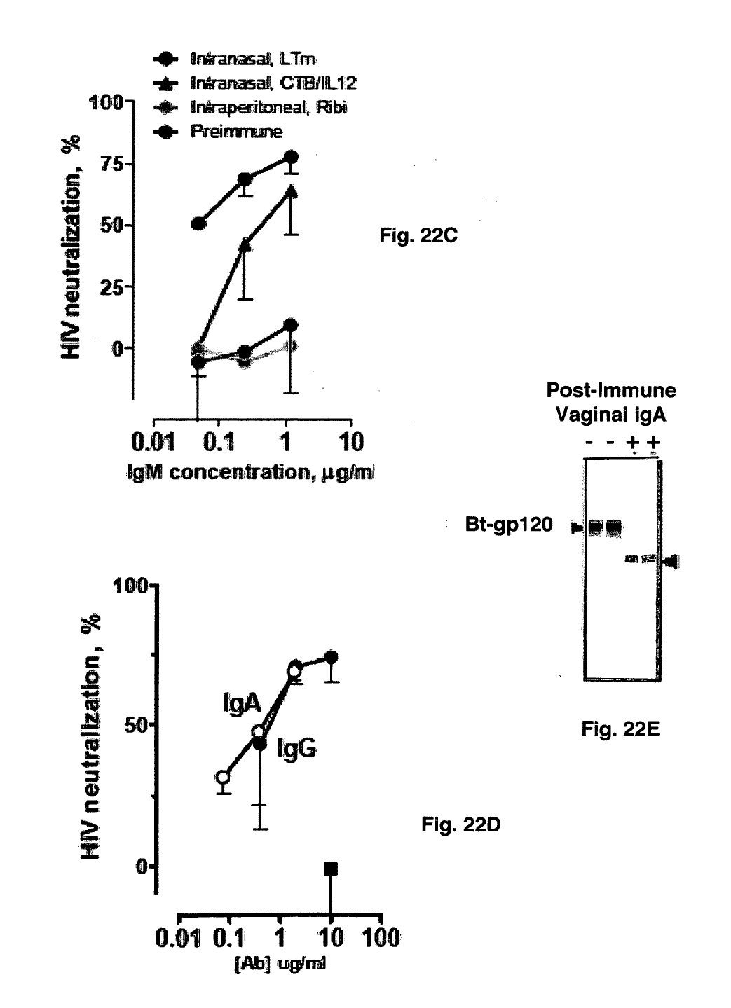

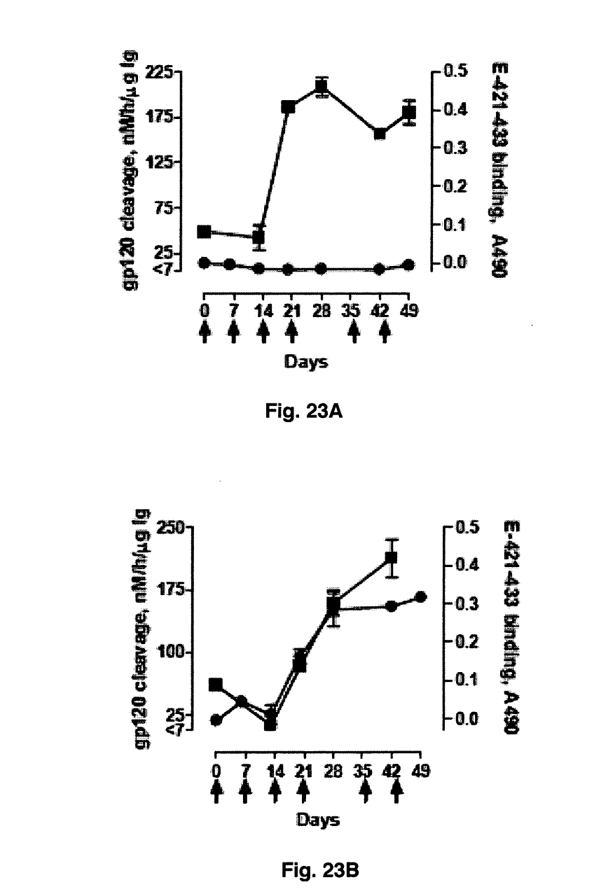

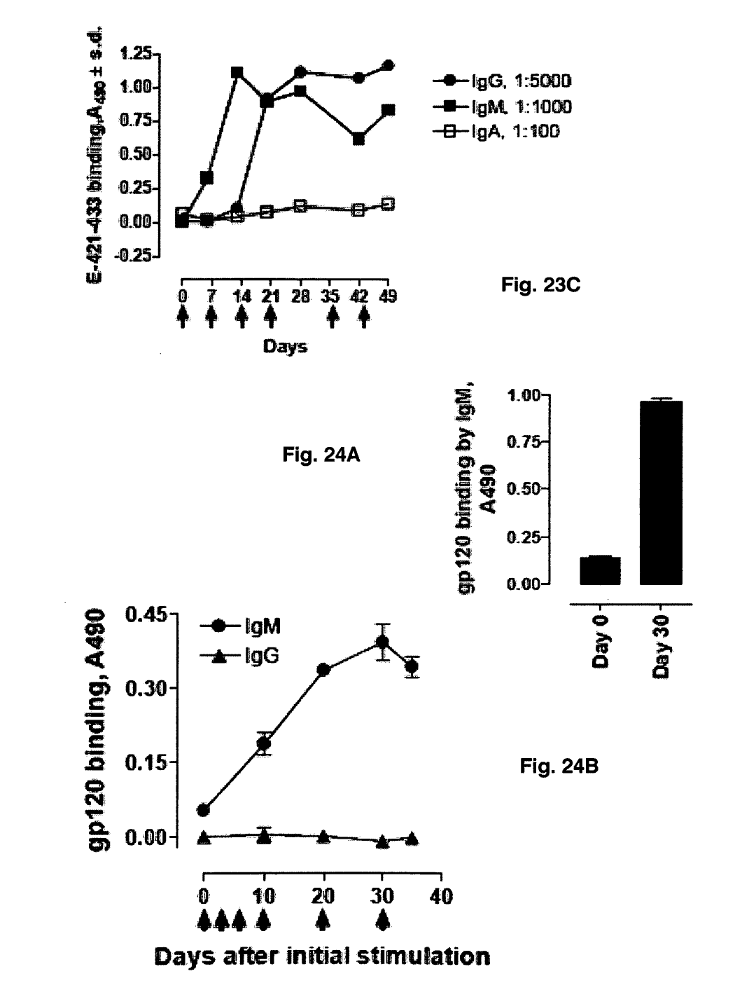

The nasal immunizations resulted in increased gp120 hydrolysis by IgM class Abs purified from the serum. In addition, strongly increased catalysis by salivary and vaginal sIgA was evident (compared to corresponding preimmune Abs; FIGS. 21 and 22A). However, the favorable effect of immunization was not a generalized one, and gp120 hydrolysis by the serum IgA and IgG was reduced compared to preimmune. Systemic immunization in Ribi resulted in reduced catalytic activities of Abs purified from blood (including IgM), and a modest increase of vaginal sIgA hydrolytic activity (FIG. 23A). E-421-433 binding Ab responses were observed in all types of immunizations in the serum, saliva and CVLF (FIGS. 22B and 23B).

The increased catalytic activity of the IgMs following nasal immunization was accompanied by strongly increased HIV neutralization (FIG. 22C). The neutralizing activities of serum IgA and IgG were essentially unchanged (FIG. 22D).

From these results, it appears that the nasal immunization with E-421-433 conjugated to KLH immunization induces favorable catalytic IgM and sIgA responses. Small, flexible peptides assume conformations that are highly dependent on their microenvironment (including contacts with carrier proteins and T cell epitopes incorporated in peptide immunogens). From the neutralizing data obtained using IgMs from the immunized mice, it may be concluded that at least some of the peptide molecules in the KLH conjugate must assume the neutralization-relevant 421-433 conformation mimicking the native conformation of this region in viral gp120.

Concerning the favorable effect of the route of immunization, nasal immunization results in antigen transport by M cells to dendritic cells followed by T cell antigen presentation and T-dependent B cell differentiation in the nasal associated lymphocyte tissue (NALT). The B cells drain through the lymphatics, eventually migrating to various lymphoid organs and effector sites, including the reproductive tract. B cells expressing IgM receptors can undergo class switch recombination (CSR) to IgA expressing cells within the mucosa under the influence of epithelia-derived cytokines. From our sIgA results, it appears that CSR occurring in mucosal tissues supports retention and improvement of the gp120 hydrolyzing activity. From the serum IgA and IgG data, it appears that CSR occurring in systemic lymphoid tissues during the classical germinal center reaction does not result in improvement of the catalytic activity. The data also raise the likelihood that different subpopulations of B cells may be responsible for synthesis of the desired neutralizing Abs. For instance B-1 cells and B-2 cells are known to produce Abs with differing antigen recognition capability, presumably because of the influence of their local milieu in which ligands responsible for activating the cells are found at varying concentrations (e.g., cytokines). An ideal HIV vaccine should preferably stimulate the synthesis of both secretory and systemic Abs with gp120 hydrolyzing and HIV neutralizing activity. Despite the obvious limitations of the immunization scheme described in the present example, it remains that these studies provide the first concrete evidence supporting the feasibility of developing a mucosal peptide-based vaccine that induces protective Abs to a conserved gp120 epitope. We conceive that the limitations of E421-433 will be overcome by the dual epitope constructs described in Example II. The dual epitope constructs are designed to stimulate a classical germinal center reaction that permits broadening of the immune response to all classes of Abs, that is, the IgM, IgG and IgA classes. Following are additional examples of immunogens that are conceived to help induce improvements in the quality of the Ab response.

Multimeric superantigenic peptide constructs: BCR cross-linking is thought to be essential for inducing robust Ab responses. Multimeric antigens can effectively achieve BCR cross-linking in the B cell membrane and induce enhanced Ab responses. This is equally true for conventional antigens and superantigens (15). An example of a multimeric gp120 peptide immunogen conceived to enhance the synthesis of protective Abs is (T-E416-433)₃. T in this construct signifies a universal T cell epitope, such as the T epitope peptide described in Example II. The E416-433 unit in (T-E-416-433)₃ is synthesized in a manner similar to E421-433 as described in our previous publication (16), i.e., condensation of the protected T416-431 and diphenyl amino(4-amidinophenyl)methanephosphonate (Lys432-Ala433 mimic) followed by deprotection with trifluoroacetic acid. The monomeric T-E416-433 is conjugated to a commercially available multiple antigenic peptide core using a bicarboxylate linker to afford the trimeric T-E416-433. Inclusion of residues 416-419 in the peptide increases the propensity to fold into a helical conformation (17-19). The peptide formulation may further be incorporated in an emulsion composed of negatively charged and neutral phospholipids at 1:3 molar ratio (e.g., phosphatidyl glycerol/phoshatidyl choline) to induce _-helix formation. To increase peptide helicity, a metal-dependent helix nucleation sequences can be included at the N terminus, e.g., DKDGDGYISAAE (taken from calcium-binding loop of calmodulin). Upon coordination with lanthanide ions, the AAE region forms a very stable alpha-helical conformation (fully helical from 4-65° C.) and provides a helix nucleation site for peptide segments attached to its C terminus (20).
Immunizations, adjuvants and validation of Ab activity: Validation of the candidate HIV vaccine constructs is done essentially as in Example II. This entails administration of the appropriate amounts of the immunogens constructs (10-200 μg peptide equivalents/kg) to experimental animals (mice, rhesus macaques) via various routes (nasal, vaginal, oral, intramuscular, intraperitoneal, intravenous) at appropriate intervals (e.g., 2-4 weeks). Repeat immunizations are conducted to stimulate memory B cell responses. Appropriate adjuvants are coadministered with the immunogen to induce efficient B cell maturation and Ab class switching as in Example II. Tests for immunogenicity include measurement of antigen binding (the immunogen, gp120, E-gp120, intact HIV particles) by of purified Abs (IgM, IgG, IgA, sIgA) from sera, saliva and vaginal lavage fluid, tests of catalytic hydrolysis of gp120 by the Abs and tests of neutralization of diverse HIV strains in tissue culture using PBMC hosts. In vivo tests of Ab efficacy are conducted by immunization of rhesus macaques with the candidate vaccine, challenge with an appropriate SHIV strain and measurement of SHIV infection in blood cells and lymphoid tissues as in Example II.

### EXAMPLE 4

### Stimulation of Antibody Synthesis by Polyclonal B Cell Activators

Antigen binding to the antigen receptor (BCR, surface immunoglobulin complexed to signal transduction molecules) is a major pathway to clonal selection of B cells, eventually resulting in the synthesis of Abs specific for the antigen. Other ligands interact with B cell receptors such as the TLRs and stimulate the polyclonal activation of B cell subpopulations expressing these receptors at high levels. The ability to synthesize neutralizing proteolytic Abs directed to the superantigenic site of gp120 is a property of preimmune B cells (Example III). Only a subpopulation of preimmune Abs express the desired gp120 hydrolyzing and HIV neutralizing activities, indicating the individual subpopulations of preimmune B cells synthesize the desired Abs at variable levels. One embodiment of the present invention consists of stimulating the synthesis of the desired Abs by polyclonal stimulation of the B cells, regardless of whether this is accomplished by a ligand that is structurally related to gp120.

Two types of ligands are conceived to accomplish the increased synthesis of HIV neutralizing Abs in the present example: (a) superantigenic ligands other than gp120 that act through the BCR; and (b) adjuvant-class ligands that act through receptors other than the BCR, e.g., the TLRs and cytokine receptors, and stimulate the B cells in the absence of BCR-stimulating ligands.
Superantigen stimulants (SAgs). The known B cell SAgs (e.g., Staphylococcal Protein A, Peptostreptooccal Protein L) do not express appreciable sequence similarity to gp120. However, like the SAg site of gp120, the Protein A SAg site is recognized by preferentially by VH3+ family immunoglobulins (Igs) and recognition of the SAg sites of both proteins is dominated by contacts at the framework regions (FRs). Here, we describe the unexpected observation that administration of Protein A stimulated the formation of Abs that recognize gp120. This is important because it provides a novel means to bypass the barriers to adaptive improvement of protective Abs directed to the to gp120 SAg site. For example, administration of Protein A to humans is conceived to enhance the synthesis of protective Abs to gp120 found in the preimmune repertoire and thereby increase the level of immunological resistance to HIV infection.

(a) gp120 binding by IgM antibodies raised by administration of Protein A. Intraperitoneal administration of Protein A without adjuvant on days 0, 3, 6, 9, 20 and 30 resulted in increased gp120 binding by the IgM fraction (FIG. 24A). Preimmune IgM showed weak, but significant gp120 binding signals (FIG. 24A, day 0). Administration of Protein A mixed with RIBI resulted in a gp120-binding IgM response similar to Protein A administered in phosphate buffered saline (PBS). gp120 binding by the IgG fractions remained undetectable after Protein A administration (FIG. 24B). The IgM response to Protein A was dose-dependent, with greatest gp120 binding IgMs observed using 1 mg/injection and little or no response at 20 μg Protein A/injection (administered according to the schedule indicated by arrows in FIG. 24B). The gp120 binding IgM response was also observed when the Protein A was administered by the intravenous route. Iodination of Protein A abrogates its Fc binding activity (1). Immunization with iodinated Protein A (Protein Aᵢ; prepared as in our previous studies, ref 5) also resulted in increased gp120 binding IgMs, indicating that the Fc binding activity of the protein was not a factor in the observed immune response. The binding of Protein Aᵢ by the IgM fraction tended to decrease below preimmune IgM levels at early time points (3 days) and was restored to the preimmune levels by day 6.

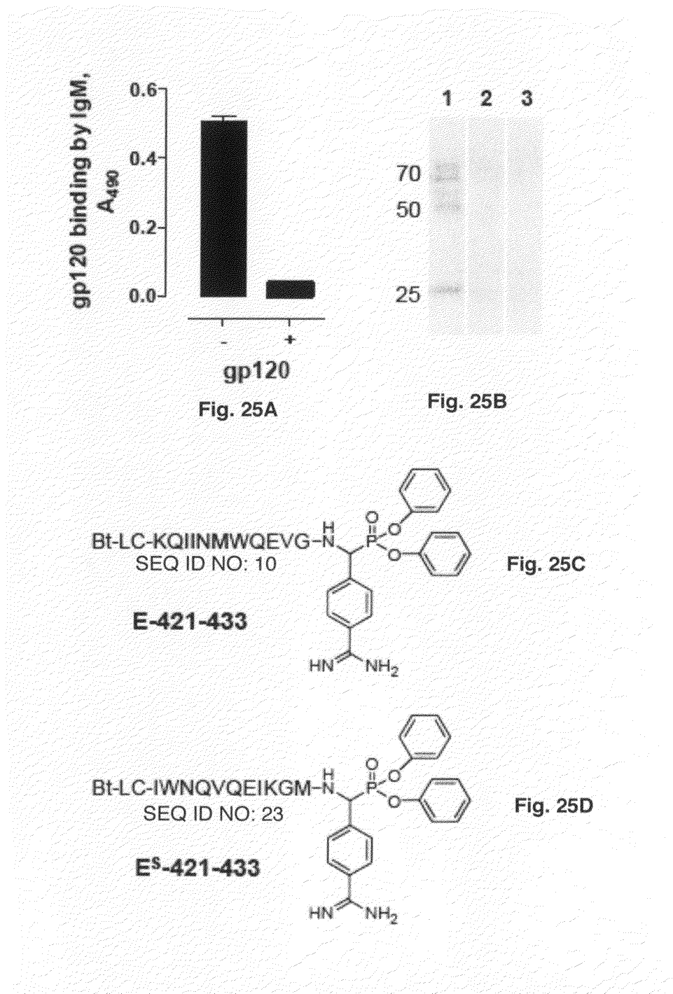

Binding of immobilized gp120 by the Protein A-induced IgMs was virtually completely inhibited by soluble gp120 (FIG. 25A), indicating a saturable reaction. The binding was observed in the presence of excess irrelevant proteins (1% skim milk), indicating that the recognition of gp120 by the IgMs is specific phenomenon. Recognition of the SAg gp120 peptides 421-433 was studied by an electrophoresis assay using the electrophilic phosphonate analog of the peptide (E-421-433; synthesis and Ab reactivity of this probe are described in ref (2, 3) and preceding Examples). The assay relies on noncovalent recognition of the antigen followed by covalent binding of the electrophile at Ab nucleophilic sites. As reported previously (3), such nucleophilic sites are present in all Abs, regardless of their catalytic activity. As shown in FIG. 25B, E-421-433 formed complexes with the IgMs induced by administration of Protein A at levels above control electrophilic probes containing the shuffled 421-433 sequence (E^{S}-421-433; FIG. 25C) or no peptide sequence (E-hapten; for E-hapten structure, see ref (3)). It may be concluded from these observations that Protein A stimulates the synthesis of Abs directed to the SAg site of gp120.

(b) Shared structural basis for B cell superantigenic properties of Staphylococcal Protein A and HIV gp120. Crystallography studies indicate that the SAg site of Protein A is located in helices II and III of domain D, and the SAg site binds Abs by establishing contacts at the following V_{H} domains framework residues: Gly-H15, Ser-H17, Arg-H19, Lys-H57, Tyr-H59, Lys-H64, Gly-H65, Arg-H66, Thr-H68, Ser-H70, Gln-H81, Asn-H82a and Ser-H82b (PDB, IDEE; (4)). Studies by our group (5, 6) and other investigators (7, 8) have indicated that the 421-433 gp120 region is an essential component of the B cell SAg site expressed by this protein. Six V_{H} framework residues important in Protein SAg binding (bold above) have been deduced to be critical for Ab binding to the gp120 SAg site (shown in red in FIG. 26A; (9)). The cognate Protein A residues that interact with the six V_{H} residues are clustered in helix II (Gln-32, Ser-33, Asp-37). Circular dichroism and NMR studies have revealed that the gp120₄₂₁₋₄₃₃ region can adopt two different secondary structures, _-sheet and _-helix, and the _-helical form has been suggested to be dominant in the CD4 receptor-bound state (10-12). The _-helical form of gp120₄₂₁₋₄₃₃ is superimposable on helix II of Protein A SAg site (root mean square deviation for C₁-_ carbons, 0.88 Å; FIG. 26B). In the superimposed model, the side chains of Gln-8 and Glu-9 in gp120₄₂₁₋₄₃₃ are located in close proximity to the Protein A helix II residues responsible for establishing contacts with Abs (Gln32 and Ser33), and the Ala13 carbonyl of gp120₄₂₁₋₄₃₃ is located near the Asp37 _-carboxyl of Protein A helix II. These considerations suggest that the SAg determinants of Protein A and gp120 contain common structural elements.

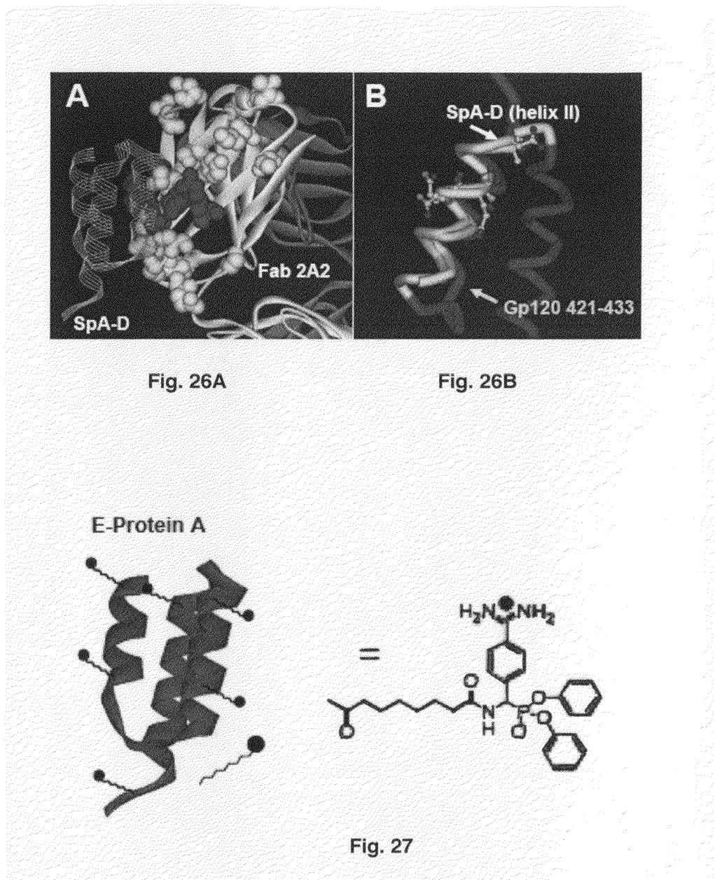

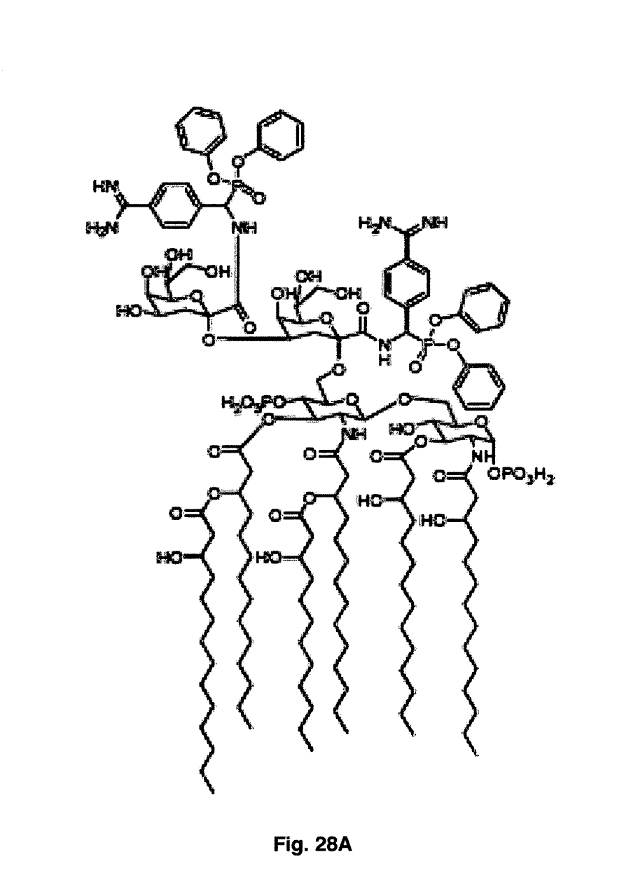

(c) E-Protein A stimulants: Like Protein A, the electrophilic derivative of Protein A is conceived to induce gp120 recognizing Abs. Like the electrophilic derivatives of gp120, electrophilic derivatives of Protein A are conceived to induce the synthesis of Abs with enhanced nucleophilic reactivity, thereby imparting to the Abs the ability to bind gp120 with covalent character and catalyze the hydrolysis of gp120. The presence of repeat epitopes capable of multivalent interactions with BCRs may facilitate productive stimulation of B cells. E-Protein A and control Protein A devoid of the electrophilic group E (FIG. 27) are utilized as the stimulants. Preparation of E-Protein A is done, for example, by the methods used for E-gp120 and E-421-433 (3, 13), that is, acylation of Lys side chain amino groups with the phosphonate containing precursor group. Phosphonate incorporation (mol phosphonate/mol protein) is determined by measuring the residual amino groups with fluorescamine. A suitable starting material is recombinant Protein A (expressed in E. coli). Smaller fragments of Protein A containing its SAg site and engineered multimers of these fragments can also be used. E-Protein A are studied as immune stimulants in groups of 5 BALB/c mice each. Adjuvants that influence the quality and magnitude of Ab responses and induce class switching from IgM to IgA and IgG are tested (14). No adjuvant is necessary for induction of the gp120 binding IgMs following Protein A administration. For example, the adjuvants/costimulators LPS, CD40L, IL4, C3dg and IL12 can be useful to enhance the immune response to T-independent antigens and help induce class switching. Administration of E-Protein A is conducted, for example, on days 0, 2, 5, 10 and 20 days (e.g., using 50 and 100 μg dose). Protein A and E-Protein A can be conceived as immune stimulants that help overcome the initial barrier to mounting protective Ab responses to the SAg site of gp120. The administration of E-Protein A or Protein A to the experimental animals can be combined with the immunogens described in Examples I, II and III to amplify the synthesis of the anti-gp120 Abs and improve their specificity for HIV. For example, the dual epitope polypeptide constructs of Example II can be coadministered with the E-Protein A or administered after the initial E-Protein A administration to increase the magnitude and specificity of the Ab response.

Validation of the candidate immune stimulants is done essentially as in Example II by study of gp120 and HIV binding by the Abs present in various biological fluids, tests of catalytic hydrolysis of gp120 by the Abs and tests of neutralization of diverse HIV strains in tissue culture using PBMC hosts. In vivo tests of immune stimulant efficacy can be conducted using rhesus macaques challenged with a SHIV strain as in Example II.
Adjuvant-class ligands. Direct polyclonal activation of B cells independent of BCR is conceived to stimulate the pre-existing capability of the cells to synthesize protective Abs to the SAg site of gp120. Classical examples of this type of polyclonal B cell activation are provided by substances commonly classified as adjuvants. For example, stimulation of splenocytes with CpG results in 5-fold increase of IgM-producing cells (15). These substances can be homogeneous formulations or complex mixtures of microbial products. Certain bacterial toxins, such as heat labile E. coli enterotoxin and cholera toxin, and their less toxic analogs are polyclonal B cell activators. Recent studies have demonstrated that Toll-like receptors (TLRs) are the critical link between the innate and the adaptive immunity. Well-defined synthetic TLR ligands have been identified as vaccine adjuvants. Examples include imidazoquinolines (TLR7 agonists), CpG oligodeoxynucleotides (TLR9 agonists) and lipid A analogues (TLR4 agonists). These compounds directly activate B cells and plasmacytoid dendritic cells, thereby promoting B cell proliferation and the maturation/activation of professional antigen presenting cells.
In addition to underivatized polyclonal B cell stimulants, electrophilic analogs of these substances (E-stimulants) are conceived to be useful in the present invention to amplify the synthesis of anti-HIV protective Abs. The E-stimulants are conceived to bind their receptors expressed on B cells irreversibly. This follows from our observations that various non-enzymatic receptor proteins can express nucleophilic sites (16). The irreversible binding precludes dissociation of the ligand from the receptor. This is conceived to impart superior biological potency to the E-stimulants compared to their reversibly binding counterparts. The E-stimulants can be administered to a living organism alone or in combination with the immunogens described in Examples I, II and III to obtain a superior protective response to HIV. Examples of the E-stimulants follow.

(a) E-LTm: LTm is a R192G mutant of heat labile E. coli enterotoxin known to stimulate vigorous mucosal Ab responses (17). The mutation renders the protein non-toxic by knocking out the ability to interact with GS_-mediated signal transduction. Introduction of the electrophilic groups can be done by the same method as E-gp120 preparation (13), i.e., acylation of LTm with the N-hydroxysuccinimide ester of diphenyl suberoylamino(4-amidinophenyl)methanephosphonate. The resultant E-LTm can then be purified by an appropriate chromatographic method.

(b) E-PWM: Pokeweed mitogen (PWM) is a lectin with mitogenic activities, extracted from roots of pokeweed, Phytolacca americana. PWM induces polyclonal immunoglobulin production in human lymphocytes. Introduction of the electrophilic groups into PWM can be done by the same method as E-gp120.

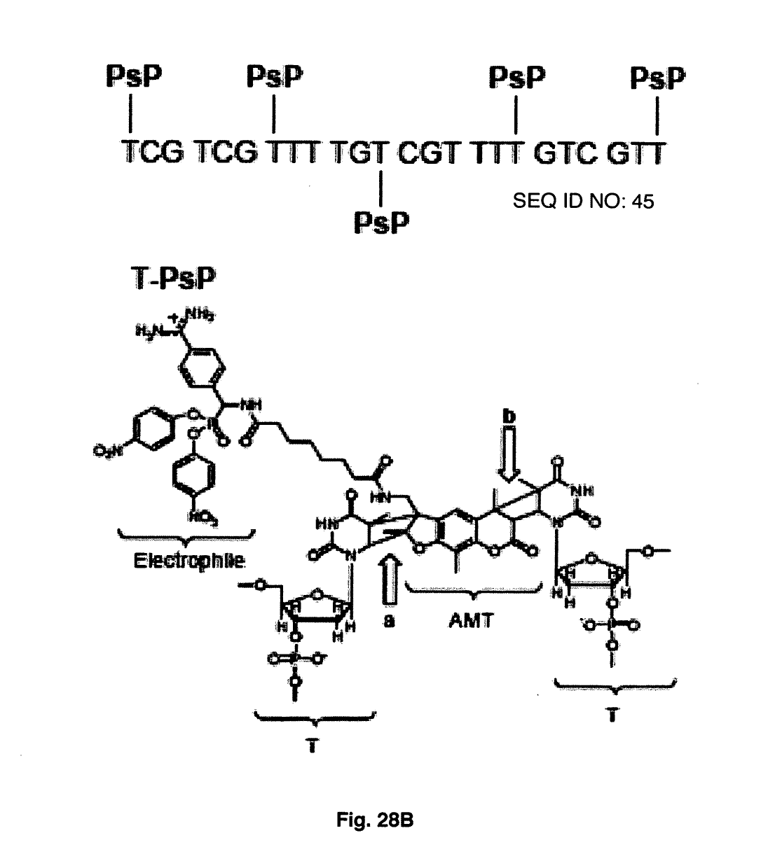

(c) E-LPS: LPS is a lipid A analog that stimulates T-dependent and independent responses. Shown in FIG. 28A is an example E-LPS derived from E. coli Re LPS. Introduction of the electrophilic groups into LPS can be done by the condensation of LPS and diphenyl amino(4-amidinophenyl)methanephosphonate.

(d) E-CpG: Shown in FIG. 28B is an example E-CpG derived from CpG ODN2006, a TLR9 agonist. Introduction of the electrophilic groups into CpG can be done by photo-activated covalent addition of aminomethylpsoralen followed by acylation with the N-hydroxysuccinimide ester of di(4-nitrophenyl) suberoylamino(4-amidinophenyl)methanephosphonate.

The foregoing stimulants can be administered to a living organism alone or together with the immunogens described in Examples I, II and III to amplify the synthesis of the anti-gp120 Abs and improve their specificity for HIV. For example, the dual epitope polypeptide constructs of Example II can be coadministered with the E-LTm or administered after the initial E-LTm administration to increase the magnitude and specificity of the Ab response.

Validation of the candidate stimulants is done essentially as in Example II by study of gp120 and HIV binding by the Abs present in various biological fluids, tests of catalytic hydrolysis of gp120 by the Abs and tests of neutralization of diverse HIV strains in tissue culture using PBMC hosts. In vivo tests of stimulant efficacy can be conducted using rhesus macaques challenged with a SHIV strain as in Example II.

### EXAMPLE 5

### Isolation of Homogeneous Binary Epitope Specific Antibodies to gp120

Abs with potent and cross-clade HIV neutralizing activity can be administered to HIV infected patients as passive immunotherapeutic reagents. In addition topical application of the Abs in the vagina or rectum prior to sexual intercourse can be applied to block transmission of HIV. The binary epitope specific Abs described in Example I display the ability to neutralize diverse HIV strains and are prototypical reagents with potential clinical use. The present Example describes the selection and screening of large numbers of Abs using the dual epitope polypeptide analogs of Example II as the means to identify high potency HIV neutralizing Abs.

Source of Abs. In one embodiment of the invention, the Ab source is experimental animals or humans immunized with the polypeptide immunogens described in Examples I and II. These immunogens contain the superantigen (SAg) epitope recognized mainly at the Ab framework regions (FRs) along with at least one other epitope that can be recognized by the complementarity determining regions (CDRs). In Examples I and II, the immunization procedures are conceived to stimulate vigorous the synthesis of Abs with binary epitope specificity. Using methods that are known in the art and described briefly below, homogeneous preparations of Abs with the binary epitope specificity and HIV neutralizing activity can readily be isolated using B cells from the immunized experimental animals or humans.
Selection from phage Ab libraries. Ref (1) describes example methods for the preparation and characterization of the phage libraries expressing single chain Fv (scFv) constructs cloned from the expressed Ab repertoires of humans and mice. The cDNA for the scFv constructs is obtained by reverse-transcriptase-polymerase reaction amplification of Ab mRNA from B cells. scFv constructs are composed of Ab VL and VH domains linked by a short and flexible peptide linker. Like noncatalytic Abs, peptidase Abs are capable of binding antigens with high specificity mediated by contacts at residues from the VL and VH domains (2). Other Ab fragment repertoires can also be generated using similar methods, e,g., light chain subunit repertoires (1). scFv repertoires composed of as many as 10⁸-10⁹ clones can be prepared and displayed on the surface of phage particles, and ribosome display libraries permit display of even large numbers of Ab fragments. Other display methods such as bacterial and yeast display can also be applied to identify the desired Abs.

Once the phage displayed repertoire has been constructed, the phage particles are contacted with the dual epitope polypeptides to select and isolate phage scFv constructs with binary epitope specificity. The dual epitope polypeptide selection reagent can be, for example, (301-311)-GMB-GGS-(E-421-433) or other dual epitope polypeptides described in Examples I and II. The inclusion of the electrophilic phosphonate group in the selection reagent is optional, and permits isolation of scFv fragments that bind gp120 with covalent character or catalyze the hydrolysis of gp120. Methods for phage selection are similar to those described in our publications (3, 4). For example, we have isolate gp120 specific catalysts using the E-421-433 and full-length E-gp120 described in Example I. The selection reagents capture specific catalysts by combining the covalent bonding reaction with traditional noncovalent bonding occurring at the epitope-paratope interface. Using E-421-431, we isolated a gp120 hydrolyzing specific Ab light chain fragment from our human phage library (4). A biotin group is included in the phage selection reagent. Phage-probe complexes are trapped on a streptavidin column and then eluted by cleaving the S—S bond located between the biotin and the phosphonate moieties. Purified preparations of the scFv or other Ab fragments are obtained by metal-affinity chromatography. Catalysis assays can utilize various substrates, including gp120, intact HIV particles or gp120 synthetic peptides containing a reporter fluorescent group. Specificity is confirmed by lack of cleavage of irrelevant polypeptides studied in parallel (albumin, extracellular domain of EGFR). In the published example of a gp120 hydrolyzing light chain isolated by these procedures, the light chain displayed the ability to bind E-421-433 covalently. Synthetic 421-436 peptide devoid of the electrophilic phosphonate moiety inhibited the covalent binding of E-421-433, suggesting that the catalytic nucleophile is located close to the site responsible for noncovalent recognition. gp120 hydrolyzing scFv constructs have been isolated from a previously described human scFv library (I) using full-length E-gp120 as the phage selection reagent. In this case, immobilized anti-biotin Ab was used to capture phages complexed with E-gp120, followed by a low pH elution step to elute the phages. Twenty four purified scFv fragments obtained from the bound phage fraction were screened for cleavage of biotinylated gp120 by electrophoresis. Eight catalytic scFv clones were identified (International Patent Application Number PCT/US2004/009398 and Publication Number WO2004/087735).

Once the binary epitope specific Ab fragments are obtained from the phage libraries, they can be transferred to vectors expressing the appropriate Ab constant domains to obtain full-length Abs by standard antibody engineering methods (5). The full-length binary epitope specific Abs can be of any desired class, e.g., IgG, IgA or IgM. The vectors are available commercially, for example, from Lonza. The vectors contain human Ab constant domains flanked by restriction sites for insertion of foreign V domains. The constant domains bring to Abs certain effector functions, for example, the ability to fix complement, mediate Ab-dependent cellular cytotoxicity and bind Fc receptors expressed on antigen presenting cells. The feasibility of obtaining therapeutic grade Abs by these methods is shown by the development of a human scFv construct against tumor necrosis factor using a phage library prepared from unimmunized human subjects. Recloned as full-length IgG, this construct has been recently approved for the treatment of rheumatoid arthritis (6).

MONOCLONAL Abs from B cells. Full-length Abs can also be cloned directly from the B cells of living organisms, e.g., humans immunized with the dual epitope polypeptide constructs of Examples I and II. For example, the Abs can be using a lymphocyte selection technique that identifies cells that bind a dual epitope construct binary. The lymphocytes could be obtained from peripheral blood cells or mucosal tissues, e.g., tonsils or gut-associated lymphoid tissue (from tonsillectomy for other reasons or from cadavers at autopsy). Peripheral blood offers the advantage of assured cell viability and ready availability. Mucosal B lymphocytes undergo recirculation and migration to distant locations. For example, intranasal immunizations induce readily detectable sIgAs/IgAs in the blood and distant mucosal sites, including the vagina (7, 8). The blood is likely to contain at least a sub-population of the lymphocytes that recirculate from mucosal tissues. As the proposed B cell selection method can pick up rare B lymphocytes producing the desired Abs, blood from a well-characterized donor expressing the desired binary epitope specific Abs in serum can be used as the source of the monoclonal Abs.

As an example, B cells are isolated from the peripheral blood (200 ml) by Ficoll-Hypaque fractionation and negative selection procedures on magnetic beads (B cell isolation kit, Miltenyi Biotec). To isolate the desired B cells, the preparation are incubated (60 min) with the dual epitope selection reagent, for example, biotinylated (301-311)-GMB-GGS-(E-421-433) or other dual epitope polypeptides described in Examples I and II, followed by flow cytometric analysis for staining with streptavidin-peroxidase. An irrelevant electrophilic peptide probe (e.g., E-VIP) is used to define the level of background reactivity. Cells displaying the highest staining with the dual epitope selection reagent (top 1% stained cells; ˜10³ cells) are sorted by flow cytometry for monoclonal Ab production. An alternative to flow sorting is the use of streptavidin-coated magnetic beads, but this procedure provides lesser control over the identification of cells with the greatest SAg reactivity. The cells are then immortalized by a recently-published improved EBV transformation method (9). Briefly, the cells are distributed in 96-well plates (e.g., 10 cells/well; 500 wells) and transformed with Epstein Barr virus (EBV, 30% supernatant of infected B95 cells) in the presence of irradiated feeder PBMCs, cyclosporin A and CpG2006 in 10% Ig-depleted FCS. In 24 weeks, supernatants can be screened for production of the desired Abs. Only 50-60% of the wells display growth, assuring a reasonable probability that the wells contain monoclonal cells. Expression levels are up to 12 μg Ig/ml using a dot blot assay. Screening is done, for example, using IgMs from supernatants purified on immobilized anti-IgM columns. The purified IgMs are incubated with Bt-gp120 and subjected to SDS-electrophoresis for detection of the cleavage reaction. As EBV-transformed B cells can display loss of Ab production (10), the V domain genes from interesting binary epitope specific Abs are rescued using a reverse-transcriptase-PCR reaction (RT-PCR) and cloned into appropriate mammalian expression vectors to provide a stable source of the Abs.

Improvement of Abs by engineering. Various protein engineering methods are available to improve Ab binding affinity, catalytic activity and HIV neutralizing activity. These are described extensively in the literature (e.g., International patent application number PCT/US2004/009398 and publication number WO2004/087735) and can be applied as needed to improve the functional properties of the binary epitope specific Abs. For example, increased avidity of HIV-1 recognition can be obtained by forming multimers of the scFv. Tetravalent antibody fragments can be generated by placing a 33-amino acid self-aggregating peptide derived from the GNC4 protein at the C terminus of an scFv construct (11). The length and constitution of the linker peptide can varied to improve VL-VH interfacial pairing in scFv constructs. The linker methodology can also be applied to generate bispecific antibodies, i.e., antibodies comprised of two scFv components with differing antigenic specificity. In this instance, the goal is to target two distinct antigens, e.g., a bispecific construct directed to the transferrin receptor and CD3 is shown to direct CD3+ T cells to lyse cells expressing the transferrin receptor. Improvements can also be achieved by introduction of random mutations in the CDRs using mutagenic primers followed by phage selection to identify the best Abs with binary epitope specificity. Alternatively, favorable mutations can be introduced in the V domains on a rational basis to improve binding and catalytic activities, particularly if structural information is available about the antigen-antibody complex. For instance, candidate amino acids suitable for mutagenesis can be identified by molecular modeling or X-ray crystallography information. Molecular modeling of antibody V domains is carried out using combined homology and ab initio algorithms, and computer programs with strong predictive value for tracing peptide backbone topography are available.

### REFERENCES FOR EXAMPLE 1

1. Graham, B. S., McElrath, M. J., Connor, R. I., Schwartz, D. H., Gorse, G. J., Keefer, M. C., Mulligan, M. J., Matthews, T. J., Wolinsky, S. M., Montefiori, D. C., Vermund, S. H., Lambert, J. S., Corey, L., Belshe, R. B., Dolin, R., Wright, P. F., Korber, B. T., Wolff, M. C., and Fast, P. E. (1998) Analysis of intercurrent human immunodeficiency virus type 1 infections in phase I and II trials of candidate AIDS vaccines. AIDS Vaccine Evaluation Group, and the Correlates of HIV Immune Protection Group. J Infect Dis 177, 310-319
2. Pitisuttithum, P., Berman, P. W., Phonrat, B., Suntharasamai, P., Raktham, S., Srisuwanvilai, L. O., Hirunras, K., Kitayaporn, D., Kaewkangwal, J., Migasena, S., Sheppard, H. W., Li, E., Chernow, M., Peterson, M. L., Shibata, R., Heyward, W. L., and Francis, D. P. (2004) Phase I/II study of a candidate vaccine designed against the B and E subtypes of HIV-1. J Acquir Immune Defic Syndr 37, 1160-1165
3. Gorny, M. K., Xu, J. Y., Karwowska, S., Buchbinder, A., and Zolla-Pazner, S. (1993) Repertoire of neutralizing human monoclonal antibodies specific for the V3 domain of HIV-1 gp120. J Immunol 150, 635-643
4. Wolfs, T. F., Zwart, G., Bakker, M., Valk, M., Kuiken, C. L., and Goudsmit, J. (1991) Naturally occurring mutations within HIV-1 V3 genomic RNA lead to antigenic variation dependent on a single amino acid substitution. Virology 185, 195-205
5. Arendrup, M., Sonnerborg, A., Svennerholm, B., Akerblom, L., Nielsen, C., Clausen, H., Olofsson, S., Nielsen, J. O., and Hansen, J. E. (1993) Neutralizing antibody response during human immunodeficiency virus type 1 infection: type and group specificity and viral escape. J Gen Virol 74 (Pt 5), 855-863
6. Kumar, A., Lifson, J. D., Silverstein, P. S., Jia, F., Sheffer, D., Li, Z., and Narayan, O. (2000) Evaluation of immune responses induced by HIV-1 gp120 in rhesus macaques: effect of vaccination on challenge with pathogenic strains of homologous and heterologous simian human immunodeficiency viruses. Virology 274, 149-164
7. Burton, D. R., Pyati, J., Koduri, R., Sharp, S. J., Thornton, G. B., Parren, P. W., Sawyer, L. S., Hendry, R. M., Dunlop, N., Nara, P. L., and et al. (1994) Efficient neutralization of primary isolates of HIV-1 by a recombinant human monoclonal antibody. Science 266, 1024-1027
8. Posner, M. R., Cavacini, L. A., Emes, C. L., Power, J., and Byrn, R. (1993) Neutralization of HIV-1 by F105, a human monoclonal antibody to the CD4 binding site of gp120. J Acquir Immune Defic Syndr 6, 7-14
9. Zhang, M. Y., Xiao, X., Sidorov, I. A., Choudhry, V., Cham, F., Zhang, P. F., Bouma, P., Zwick, M., Choudhary, A., Montefiori, D. C., Broder, C. C., Burton, D. R., Quinnan, G. V., Jr., and Dimitrov, D. S. (2004) Identification and characterization of a new cross-reactive human immunodeficiency virus type 1-neutralizing human monoclonal antibody. J Virol 78, 9233-9242
10. Binley, J. M., Wrin, T., Korber, B., Zwick, M. B., Wang, M., Chappey, C., Stiegler, G., Kunert, R., Zolla-Pazner, S., Katinger, H., Petropoulos, C. J., and Burton, D. R. (2004) Comprehensive cross-clade neutralization analysis of a panel of anti-human immunodeficiency virus type 1 monoclonal antibodies. J Virol 78, 13232-13252
11. Kwong, P. D., Wyatt, R., Robinson, J., Sweet, R. W., Sodroski, J., and Hendrickson, W. A. (1998) Structure of an HIV gp120 envelope glycoprotein in complex with the CD4 receptor and a neutralizing human antibody. Nature 393, 648-659
12. Olshevsky, U., Helseth, E., Furman, C., Li, J., Haseltine, W., and Sodroski, J. (1990) Identification of individual human immunodeficiency virus type 1 gp120 amino acids important for CD4 receptor binding. J Virol 64, 5701-5707
13. Thali, M., Furman, C., Ho, D. D., Robinson, J., Tilley, S., Pinter, A., and Sodroski, J. (1992) Discontinuous, conserved neutralization epitopes overlapping the CD4-binding region of human immunodeficiency virus type 1 gp120 envelope glycoprotein. J Virol 66, 5635-5641
14. Thali, M., Olshevsky, U., Furman, C., Gabuzda, D., Posner, M., and Sodroski, J. (1991) Characterization of a discontinuous human immunodeficiency virus type 1 gp120 epitope recognized by a broadly reactive neutralizing human monoclonal antibody. J Virol 65, 6188-6193
15. Zolla-Pazner, S. (2004) Identifying epitopes of HIV-1 that induce protective antibodies. Nat Rev Immunol 4, 199-210
16. Goodglick, L., Zevit, N., Neshat, M. S., and Braun, J. (1995) Mapping the Ig superantigen-binding site of HIV-1 gp120. J Immunol 155, 5151-5159
17. Berberian, L., Valles-Ayoub, Y., Sun, N., Martinez-Maza, O., and Braun, J. (1991) A VH clonal deficit in human immunodeficiency virus-positive individuals reflects a B-cell maturational arrest. Blood 78, 175-179
18. Juompan, L., Lambin, P., and Zouali, M. (1998) Selective deficit in antibodies specific for the superantigen binding site of gp120 in HIV infection. Faseb J 12, 1473-1480
19. Scamurra, R. W., Miller, D. J., Dahl, L., Abrahamsen, M., Kapur, V., Wahl, S. M., Milner, E. C., and Janoff, E. N. (2000) Impact of HIV-1 infection on VH3 gene repertoire of naive human B cells. J Immunol 164, 5482-5491
20. Bessudo, A., Rassenti, L., Havlir, D., Richman, D., Feigal, E., and Kipps, T. J. (1998) Aberrant and unstable expression of immunoglobulin genes in persons infected with human immunodeficiency virus. Blood 92, 1317-1323
21. Townsley-Fuchs, J., Kam, L., Fairhurst, R., Gange, S. J., Goodglick, L., Giorgi, J. V., Sidell, N., Detels, R., and Braun, J. (1996) Human immunodeficiency virus-1 (HIV-1) gp120 superantigen-binding serum antibodies. A host factor in homosexual HIV-1 transmission. J Clin Invest 98, 1794-1801
22. Paul, S., Karle, S., Planque, S., Taguchi, H., Salas, M., Nishiyama, Y., Handy, B., Hunter, R., Edmundson, A., and Hanson, C. (2004) Naturally occurring proteolytic antibodies: selective immunoglobulin M-catalyzed hydrolysis of HIV gp120. J Biol Chem 279, 39611-39619
23. Planque, S., Mitsuda, Y., Taguchi, H., Salas, M., M-K, M., Nishiyama, Y., R, K., Okhuysen, P., M, E., Hunter, R., Sheppard, H. W., Hanson, C. V., and Paul, S. (2007) Characterization of gp120 hydrolysis by IgA antibodies from humans without HIV infection. AIDS Research and Human Retroviruses 23, in press
24. Palacios, R., Santos, J., Valdivielso, P., and Marquez, M. (2002) Human immunodeficiency virus infection and systemic lupus erythematosus. An unusual case and a review of the literature. Lupus 11, 60-63
25. Bermas, B. L., Petri, M., Berzofsky, J. A., Waisman, A., Shearer, G. M., and Mozes, E. (1994) Binding of glycoprotein 120 and peptides from the HIV-1 envelope by autoantibodies in mice with experimentally induced systemic lupus erythematosus and in patients with the disease. AIDS Res Hum Retroviruses 10, 1071-1077
26. Karle, S., Planque, S., Nishiyama, Y., Taguchi, H., Zhou, Y. X., Salas, M., Lake, D., Thiagarajan, P., Arnett, F., Hanson, C. V., and Paul, S. (2004) Cross-clade HIV-1 neutralization by an antibody fragment from a lupus phage display library. Aids 18, 329-331
27. Nishiyama, Y., Bhatia, G., Bangale, Y., Planque, S., Mitsuda, Y., Taguchi, H., Karle, S., and Paul, S. (2004) Toward selective covalent inactivation of pathogenic antibodies: a phosphate diester analog of vasoactive intestinal peptide that inactivates catalytic autoantibodies. J Biol Chem 279, 7877-7883
28. Planque, S., Taguchi, H., Burr, G., Bhatia, G., Karle, S., Zhou, Y. X., Nishiyama, Y., and Paul, S. (2003) Broadly distributed chemical reactivity of natural antibodies expressed in coordination with specific antigen binding activity. J Biol Chem 278, 20436-20443
29. Hedstrom, L. (2002) Serine protease mechanism and specificity. Chem Rev 102, 4501-4524
30. Paul, S., Planque, S., Zhou, Y. X., Taguchi, H., Bhatia, G., Karle, S., Hanson, C., and Nishiyama, Y. (2003) Specific HIV gp120-cleaving antibodies induced by covalently reactive analog of gp120. J Biol Chem 278, 20429-20435
31. Nishiyama, Y., Karle, S., Mitsuda, Y., Taguchi, H., Planque, S., Salas, M., Hanson, C., and Paul, S. (2006) Towards irreversible HIV inactivation: stable gp120 binding by nucleophilic antibodies. J Mol Recognit 19, 423-431
32. Karray, S., and Zouali, M. (1997) Identification of the B cell superantigen-binding site of HIV-1 gp120. Proc Natl Acad Sci USA 94, 1356-1360
33. Karray, S., Juompan, L., Maroun, R. C., Isenberg, D., Silverman, G. J., and Zouali, M. (1998) Structural basis of the gp120 superantigen-binding site on human immunoglobulins. J Immunol 161, 6681-6688
34. Nishiyama, Y., Mitsuda, Y., Taguchi, H., Planque, S., Hara, M., Karle, S., Hanson, C. V., Uda, T., and Paul, S. (2005) Broadly distributed nucleophilic reactivity of proteins coordinated with specific ligand binding activity. J Mol Recognit 18, 295-306
35. Buchacher, A., Predl, R., Strutzenberger, K., Steinfellner, W., Trkola, A., Purtscher, M., Gruber, G., Tauer, C., Steindl, F., Jungbauer, A., and et al. (1994) Generation of human monoclonal antibodies against HIV-1 proteins; electrofusion and Epstein-Barr virus transformation for peripheral blood lymphocyte immortalization. AIDS Res Hum Retroviruses 10, 359-369
36. Kunert, R., Ruker, F., and Katinger, H. (1998) Molecular characterization of five neutralizing anti-HIV type 1 antibodies: identification of nonconventional D segments in the human monoclonal antibodies 2G12 and 2F5. AIDS Res Hum Retroviruses 14, 1115-1128
37. Conley, A. J., Gomy, M. K., Kessler, J. A., 2nd, Boots, L. J., Ossofio-Castro, M., Koenig, S., Lineberger, D. W., Emini, E. A., Williams, C., and Zolla-Pazner, S. (1994) Neutralization of primary human immunodeficiency virus type 1 isolates by the broadly reactive anti-V3 monoclonal antibody, 447-52D. J Virol 68, 69947000
38. Gao, Q. S., Sun, M., Rees, A. R., and Paul, S. (1995) Site-directed mutagenesis of proteolytic antibody light chain. J Mol Biol 253, 658-664
39. Ramsland, P. A., Terzyan, S. S., Cloud, G., Bourne, C. R., Farrugia, W., Tribbick, G., Geysen, H. M., Moomaw, C. R., Slaughter, C. A., and Edmundson, A. B. (2006) Crystal structure of a glycosylated Fab from an IgM cryoglobulin with properties of a natural proteolytic antibody. Biochem J 395, 473-481
40. Paul, S., Tramontano, A., Gololobov, G., Zhou, Y. X., Taguchi, H., Karle, S., Nishiyama, Y., Planque, S., and George, S. (2001) Phosphonate ester probes for proteolytic antibodies. J Biol Chem 276, 2831428320
41. Paul, S., Mei, S., Mody, B., Eklund, S. H., Beach, C. M., Massey, R. J., and Hamel, F. (1991) Cleavage of vasoactive intestinal peptide at multiple sites by autoantibodies. J Biol Chem 266, 16128-16134
42. Paul, S., Volle, D. J., Beach, C. M., Johnson, D. R., Powell, M. J., and Massey, R. J. (1989) Catalytic hydrolysis of vasoactive intestinal peptide by human autoantibody. Science 244, 1158-1162

### REFERENCES FOR EXAMPLE 2

1. Karle, S., Nishiyama, Y., Taguchi, H., Zhou, Y. X., Luo, J., Planque, S., Hanson, C., and Paul, S. (2003) Carrier-dependent specificity of antibodies to a conserved peptide determinant of gp120. Vaccine 21, 1213-1218
2. Graf von Stosch, A., Kinzel, V., Pipkorn, R., and Reed, J. (1995) Investigation of the structural components governing the polarity-dependent refolding of a CD4-binding peptide from gp120. J Mol Biol 250, 507-513
3. Reed, J., and Kinzel, V. (1993) Primary structure elements responsible for the conformational switch in the envelope glycoprotein gp120 from human immunodeficiency virus type 1: LPCR is a motif governing folding. Proc Natl Acad Sci USA 90, 6761-6765
4. Reed, J., and Kinzel, V. (1991) A conformational switch is associated with receptor affinity in peptides derived from the CD4-binding domain of gp120 from HIV I. Biochemistry 30, 4521-4528
5. Kwong, P. D., Wyatt, R., Robinson, J., Sweet, R. W., Sodroski, J., and Hendrickson, W. A. (1998) Structure of an HIV gp120 envelope glycoprotein in complex with the CD4 receptor and a neutralizing human antibody. Nature 393, 648-659
6. Paul, S., Planque, S., Zhou, Y. X., Taguchi, H., Bhatia, G., Karle, S., Hanson, C., and Nishiyama, Y. (2003) Specific HIV gp120-cleaving antibodies induced by covalently reactive analog of gp120. J Biol Chem 278, 20429-20435
7. Lin, L., Cook, D. N., Wiesehahn, G. P., Alfonso, R., Behrman, B., Cimino, G. D., Corten, L., Damonte, P. B., Dikeman, R., Dupuis, K., Fang, Y. M., Hanson, C. V., Hearst, J. E., Lin, C. Y., Londe, H. F., Metchette, K., Nerio, A. T., Pu, J. T., Reames, A. A., Rheinschmidt, M., Tessman, J., Isaacs, S. T., Wollowitz, S., and Corash, L. (1997) Photochemical inactivation of viruses and bacteria in platelet concentrates by use of a novel psoralen and long-wavelength ultraviolet light. Transfusion 37, 423-435
8. Griffiths, A. D., Williams, S. C., Hartley, O., Tomlinson, I. M., Waterhouse, P., Crosby, W. L., Kontermann, R. E., Jones, P. T., Low, N. M., Allison, T. J., and et al. (1994) Isolation of high affinity human antibodies directly from large synthetic repertoires. Embo J 13, 3245-3260
9. Planque, S., Bangale, Y., Song, X. T., Karle, S., Taguchi, H., Poindexter, B., Bick, R., Edmundson, A., Nishiyama, Y., and Paul, S. (2004) Ontogeny of proteolytic immunity: IgM serine proteases. J Biol Chem 279, 14024-14032
10. Mitsuda, Y., Planque, S., Hara, M., Kyle, R., Taguchi, H., Nishiyama, Y., and Paul, S. (2007) Naturally occurring catalytic antibodies: evidence for preferred development of the catalytic function in IgA class antibodies. Mol Biotechnol 36, 113-122
11. Paul, S., Karle, S., Planque, S., Taguchi, H., Salas, M., Nishiyama, Y., Handy, B., Hunter, R., Edmundson, A., and Hanson, C. (2004) Naturally occurring proteolytic antibodies: selective immunoglobulin M-catalyzed hydrolysis of HIV gp120. J Biol Chem 279, 39611-39619
12. Nishiyama, Y., Karle, S., Mitsuda, Y., Taguchi, H., Planque, S., Salas, M., Hanson, C., and Paul, S. (2006) Towards irreversible HIV inactivation: stable gp120 binding by nucleophilic antibodies. J Mol Recognit 19, 423-431
13. McKeating, J. A., McKnight, A., and Moore, J. P. (1991) Differential loss of envelope glycoprotein gp120 from virions of human immunodeficiency virus type 1 isolates: effects on infectivity and neutralization. J Virol 65, 852-860
14. Moore, J. P., McKeating, J. A., Weiss, R. A., and Sattentau, Q. J. (1990) Dissociation of gp120 from HIV-1 virions induced by soluble CD4. Science 250, 1139-1142
15. Moore, J. P., Wallace, L. A., Follett, E. A., and McKeating, J. A. (1989) An enzyme-linked immunosorbent assay for antibodies to the envelope glycoproteins of divergent strains of HIV-1. Aids 3, 155-163
16. Karle, S., Planque, S., Nishiyama, Y., Taguchi, H., Zhou, Y. X., Salas, M., Lake, D., Thiagarajan, P., Arnett, F., Hanson, C. V., and Paul, S. (2004) Cross-clade HIV-1 neutralization by an antibody fragment from a lupus phage display library. Aids 18, 329-331
17. Planque, S., Mitsuda, Y., Taguchi, H., Salas, M., M-K, M., Nishiyama, Y., R, K., Okhuysen, P., M, E., Hunter, R., Sheppard, H. W., Hanson, C. V., and Paul, S. (2007) Characterization of gp120 hydrolysis by IgA antibodies from humans without HIV infection. AIDS Research and Human Retroviruses 23, in press
18. Harouse, J. M., Gettie, A., Eshetu, T., Tan, R. C., Bohm, R., Blanchard, J., Baskin, G., and Cheng-Mayer, C. (2001) Mucosal transmission and induction of simian AIDS by CCR5-specific simian/human immunodeficiency virus SHIV(SF162P3). J Virol 75, 1990-1995
19. Harouse, J. M., Gettie, A., Tan, R. C., Blanchard, J., and Cheng-Mayer, C. (1999) Distinct pathogenic sequela in rhesus macaques infected with CCR5 or CXCR4 utilizing SHIVs. Science 284, 816-819
20. Hsu, M., Harouse, J. M., Gettie, A., Buckner, C., Blanchard, J., and Cheng-Mayer, C. (2003) Increased mucosal transmission but not enhanced pathogenicity of the CCR5-tropic, simian AIDS-inducing simian/human immunodeficiency virus SHIV(SF162P3) maps to envelope gp120. J Virol 77, 989-998
21. Nehete, P. N., Chitta, S., Hossain, M. M., Hill, L., Bernacky, B. J., Baze, W., Arlinghaus, R. B., and Sastry, K. J. (2001) Protection against chronic infection and AIDS by an HIV envelope peptide-cocktail vaccine in a pathogenic SHIV-rhesus model. Vaccine 20, 813-825
22. Nehete, P. N., Nehete, B. P., Manuri, P., Hill, L., Palmer, J. L., and Sastry, K. J. (2005) Protection by dendritic cells-based HIV synthetic peptide cocktail vaccine: preclinical studies in the SHIV-rhesus model. Vaccine 23, 21542159

### REFERENCES FOR EXAMPLE 3

1. Gololobov, G., Sun, M., and Paul, S. (1999) Innate antibody catalysis. Mol Immunol 36, 1215-1222
2. Paul, S., Karle, S., Planque, S., Taguchi, H., Salas, M., Nishiyama, Y., Handy, B., Hunter, R., Edmundson, A., and Hanson, C. (2004) Naturally occurring proteolytic antibodies: selective immunoglobulin M-catalyzed hydrolysis of HIV gp120. J Biol Chem 279, 39611-39619
3. Planque, S., Mitsuda, Y., Taguchi, H., Salas, M., M-K, M., Nishiyama, Y., R, K., Okhuysen, P., M, E., Hunter, R., Sheppard, H. W., Hanson, C. V., and Paul, S. (2007) Characterization of gp120 hydrolysis by IgA antibodies from humans without HIV infection. AIDS Research and Human Retroviruses 23, in press
4. Frazer J K, and JD, C. (1999) Immunoglobulins: Structure and Function. In: Fundamental Immunology, 4th edition. (Paul W E, ed) Lippincott-raven, Philadelphia, pp. 37-74
5. Kerr, M. A. (1990) The structure and function of human IgA. Biochem J 271, 285-296
6. Raux, M., Finkielsztejn, L., Salmon-Ceron, D., Bouchez, H., Excler, J. L., Dulioust, E., Grouin, J. M., Sicard, D., and Blondeau, C. (1999) Comparison of the distribution of IgG and IgA antibodies in serum and various mucosal fluids of HIV type 1-infected subjects. AIDS Res Hum Retroviruses 15, 1365-1376
7. Brandtzaeg, P., Farstad, I. N., Johansen, F. E., Morton, H. C., Norderhaug, I. N., and Yamanaka, T. (1999) The B-cell system of human mucosae and exocrine glands. Immunol Rev 171, 45-87
8. Mitsuda, Y., Planque, S., Hara, M., Kyle, R., Taguchi, H., Nishiyama, Y., and Paul, S. (2007) Naturally occurring catalytic antibodies: evidence for preferred development of the catalytic function in IgA class antibodies. Mol Biotechnol 36, 113-122
9. Powers, J. C., Asgian, J. L., Ekici, O. D., and James, K. E. (2002) Irreversible inhibitors of serine, cysteine, and threonine proteases. Chem Rev 102, 4639-4750
10. Goodglick, L., Zevit, N., Neshat, M. S., and Braun, J. (1995) Mapping the Ig superantigen-binding site of HIV-1 gp120. J Immunol 155, 5151-5159
11. Karray, S., and Zouali, M. (1997) Identification of the B cell superantigen-binding site of HIV-1 gp120. Proc Natl Acad Sci USA 94, 1356-1360
12. Bessudo, A., Rassenti, L., Havlir, D., Richman, D., Feigal, E., and Kipps, T. J. (1998) Aberrant and unstable expression of immunoglobulin genes in persons infected with human immunodeficiency virus. Blood 92, 1317-1323
13. Karle, S., Nishiyama, Y., Taguchi, H., Zhou, Y. X., Luo, J., Planque, S., Hanson, C., and Paul, S. (2003) Carrier-dependent specificity of antibodies to a conserved peptide determinant of gp120. Vaccine 21, 1213-1218
14. Holmgren, J., Adamsson, J., Anjuere, F., Clemens, J., Czerkinsky, C., Eriksson, K., Flach, C. F., George-Chandy, A., Harandi, A. M., Lebens, M., Lehner, T., Lindblad, M., Nygren, E., Raghavan, S., Sanchez, J., Stanford, M., Sun, J. B., Svennerholm, A. M., and Tengvall, S. (2005) Mucosal adjuvants and anti-infection and anti-immunopathology vaccines based on cholera toxin, cholera toxin B subunit and CpG DNA. Immunol Lett 97, 181-188
15. Sasso, E. H., Silverman, G. J., and Mannik, M. (1991) Human IgA and IgG F(ab′)2 that bind to staphylococcal protein A belong to the VHIII subgroup. J Immunol 147, 1877-1883
16. Taguchi, H., Burr, G., Karle, S., Planque, S., Zhou, Y. X., Paul, S., and Nishiyama, Y. (2002) A mechanism-based probe for gp120-Hydrolyzing antibodies. Bioorg Med Chem Lett 12, 3167-3170
17. Graf von Stosch, A., Kinzel, V., Pipkorn, R., and Reed, J. (1995) Investigation of the structural components governing the polarity-dependent refolding of a CD4-binding peptide from gp120. J Mol Biol 250, 507-513
18. Reed, J., and Kinzel, V. (1993) Primary structure elements responsible for the conformational switch in the envelope glycoprotein gp120 from human immunodeficiency virus type 1: LPCR is a motif governing folding. Proc Natl Acad Sci USA 90, 6761-6765
19. Reed, J., and Kinzel, V. (1991) A conformational switch is associated with receptor affinity in peptides derived from the CD4-binding domain of gp120 from HIV I. Biochemistry 30, 4521-4528
20. Siedlecka, M., Goch, G., Ejchart, A., Sticht, H., and Bierzyski, A. (1999) Alpha-helix nucleation by a calcium-binding peptide loop. Proc Natl Acad Sci USA 96, 903-908

### REFERENCES FOR EXAMPLE 4

1. Sasso, E. H., Silverman, G. J., and Mannik, M. (1989) Human IgM molecules that bind staphylococcal protein A contain VHIII H chains. J Immunol 142, 2778-2783
2. Taguchi, H., Burr, G., Karle, S., Planque, S., Zhou, Y. X., Paul, S., and Nishiyama, Y. (2002) A mechanism-based probe for gp120-Hydrolyzing antibodies. Bioorg Med Chem Lett 12, 3167-3170
3. Planque, S., Taguchi, H., Burr, G., Bhatia, G., Karle, S., Zhou, Y. X., Nishiyama, Y., and Paul, S. (2003) Broadly distributed chemical reactivity of natural antibodies expressed in coordination with specific antigen binding activity. J Biol Chem 278, 20436-20443
4. Graille, M., Stura, E. A., Corper, A. L., Sutton, B. J., Taussig, M. J., Charbonnier, J. B., and Silverman, G. J. (2000) Crystal structure of a Staphylococcus aureus protein A domain complexed with the Fab fragment of a human IgM antibody: structural basis for recognition of B-cell receptors and superantigen activity. Proc Natl Acad Sci USA 97, 5399-5404
5. Paul, S., Karle, S., Planque, S., Taguchi, H., Salas, M., Nishiyama, Y., Handy, B., Hunter, R., Edmundson, A., and Hanson, C. (2004) Naturally occurring proteolytic antibodies: selective immunoglobulin M-catalyzed hydrolysis of HIV gp120. J Biol Chem 279, 39611-39619
6. Nishiyama, Y., Karle, S., Planque, S., Taguchi, H., and Paul, S. (2007) Antibodies to the superantigenic site of HIV-1 gp120: hydrolytic and binding activities of the light chain subunit. Mol Immunol 44, 2707-2718
7. Goodglick, L., Zevit, N., Neshat, M. S., and Braun, J. (1995) Mapping the Ig superantigen-binding site of HIV-1 gp120. J Immunol 155, 5151-5159
8. Karray, S., and Zouali, M. (1997) Identification of the B cell superantigen-binding site of HIV-1 gp120. Proc Natl Acad Sci USA 94, 1356-1360
9. Karray, S., Juompan, L., Maroun, R. C., Isenberg, D., Silverman, G. J., and Zouali, M. (1998) Structural basis of the gp120 superantigen-binding site on human immunoglobulins. J Immunol 161, 6681-6688
10. Reed, J., and Kinzel, V. (1991) A conformational switch is associated with receptor affinity in peptides derived from the CD4-binding domain of gp120 from HIV 1. Biochemistry 30, 4521-4528
11. Reed, J., and Kinzel, V. (1993) Primary structure elements responsible for the conformational switch in the envelope glycoprotein gp120 from human immunodeficiency virus type 1: LPCR is a motif governing folding. Proc Natl Acad Sci USA 90, 6761-6765
12. Graf von Stosch, A., Kinzel, V., Pipkorn, R., and Reed, J. (1995) Investigation of the structural components governing the polarity-dependent refolding of a CD4-binding peptide from gp120. J Mol Biol 250, 507-513
13. Paul, S., Planque, S., Zhou, Y. X., Taguchi, H., Bhatia, G., Karle, S., Hanson, C., and Nishiyama, Y. (2003) Specific HIV gp120-cleaving antibodies induced by covalently reactive analog of gp120. J Biol Chem 278, 20429-20435
14. Hunter, R. L. (2002) Overview of vaccine adjuvants: present and future. Vaccine 20 Suppl 3, S7-12
15. Klinman, D. M., Yi, A. K., Beaucage, S. L., Conover, J., and Krieg, A. M. (1996) CpG motifs present in bacteria DNA rapidly induce lymphocytes to secrete interleukin 6, interleukin 12, and interferon gamma. Proc Natl Acad Sci USA 93, 2879-2883
16. Nishiyama, Y., Mitsuda, Y., Taguchi, H., Planque, S., Hara, M., Karle, S., Hanson, C. V., Uda, T., and Paul, S. (2005) Broadly distributed nucleophilic reactivity of proteins coordinated with specific ligand binding activity. J Mol Recognit 18, 295-306
17. Dickinson, B. L., and Clements, J. D. (1995) Dissociation of Escherichia coli heat-labile enterotoxin adjuvanticity from ADP-ribosyltransferase activity. Infect Immun 63, 1617-1623

### REFERENCES FOR EXAMPLE V

1. Paul, S., Tramontano, A., Gololobov, G., Zhou, Y. X., Taguchi, H., Karle, S., Nishiyama, Y., Planque, S., and George, S. (2001) Phosphonate ester probes for proteolytic antibodies. J Biol Chem 276, 28314-28320
2. Sun, M., Gao, Q. S., Kirnarskiy, L., Rees, A., and Paul, S. (1997) Cleavage specificity of a proteolytic antibody light chain and effects of the heavy chain variable domain. J Mol Biol 271, 374-385
3. Karle, S., Planque, S., Nishiyama, Y., Taguchi, H., Zhou, Y. X., Salas, M., Lake, D., Thiagarajan, P., Arnett, F., Hanson, C. V., and Paul, S. (2004) Cross-clade HIV-1 neutralization by an antibody fragment from a lupus phage display library. Aids 18, 329-331
4. Nishiyama, Y., Karle, S., Planque, S., Taguchi, H., and Paul, S. (2007) Antibodies to the superantigenic site of HIV-1 gp120: hydrolytic and binding activities of the light chain subunit. Mol Immunol 44, 2707-2718
5. McLean, G. R., Nakouzi, A., Casadevall, A., and Green, N. S. (2000) Human and murine immunoglobulin expression vector cassettes. Mol Immunol 37, 837-845
6. van de Putte, L. B., Rau, R., Breedveld, F. C., Kalden, J. R., Malaise, M. G., van Riel, P. L., Schattenkirchner, M., Emery, P., Burmester, G. R., Zeidler, H., Moutsopoulos, H. M., Beck, K., and Kupper, H. (2003) Efficacy and safety of the fully human anti-tumour necrosis factor alpha monoclonal antibody adalimumab (D2E7) in DMARD refractory patients with rheumatoid arthritis: a 12 week, phase II study. Ann Rheum Dis 62, 1168-1177
7. Imai, Y., Ishikawa, T., Tanikawa, T., Nakagami, H., Maekawa, T., and Kurohane, K. (2005) Production of IgA monoclonal antibody against Shiga toxin binding subunits employing nasal-associated lymphoid tissue. J Immunol Methods 302, 125-135
8. Shimoda, M., Inoue, Y., Ametani, A., Fujiwara, J., Tsuji, N. M., Kurisaki, J., Azuma, N., and Kanno, C. (1998) Anti-DNA IgA autoantibodies are spontaneously generated in mouse Peyer's patches. Immunology 95, 200-207
9. Traggiai, E., Becker, S., Subbarao, K., Kolesnikova, L., Uematsu, Y., Gismondo, M. R., Murphy, B. R., Rappuoli, R., and Lanzavecchia, A. (2004) An efficient method to make human monoclonal antibodies from memory B cells: potent neutralization of SARS coronavirus. Nat Med 10, 871-875
10. Pasha, R. P., Roohi, A., and Shokri, F. (2003) Establishment of human heterohybridoma and lymphoblastoid cell lines specific for the Rh D and C antigens. Transfus Med 13, 83-92
11. Pack, P., Muller, K., Zahn, R., and Pluckthun, A. (1995) Tetravalent miniantibodies with high avidity assembling in Escherichia coli. J Mol Biol 246, 28-34

Any patents or publications mentioned in this specification are indicative of the levels of those skilled in the art to which the invention pertains. These patents and publications are incorporated by reference herein to the same extent as if each individual publication was incorporated by reference specifically and individually. One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A polypeptide, or electrophilic analog thereof, comprising:
a superantigenic epitope and at least one other epitope effective to induce the production of antibodies with binary specificity for the epitopes on a polypeptide antigen.

2. The polypeptide or electrophilic analog thereof of claim 1, wherein the electrophilic analog is covalently oligomerized gp120.

3. The polypeptide or electrophilic analog thereof of claim 1, wherein the polypeptide or electrophilic analog thereof comprises superantigenic gp120 epitope linked to another gp120 epitope by a peptide linker.

4. The polypeptide or electrophilic analog thereof of claim 3, wherein the superantigen epitope comprises amino acid residues 421-433 of gp120 and the other epitope comprises amino acid residues 301-311 of gp120.

5. The polypeptide or electrophilic analog thereof of claim 3, wherein the peptide linker has a sufficient number of amino acids such that the linker length approximates the distance between the two epitopes in native gp120.

6. The polypeptide or electrophilic analog thereof of claim 1, wherein one or both epitopes further comprise one or more electrophilic groups therewithin, said electrophilic groups effective to induce synthesis of the binary specific antibodies.

7. The polypeptide or electrophilic analog thereof of claim 6, wherein one or both of the epitopes comprising the electrophilic analog has the structural formula: 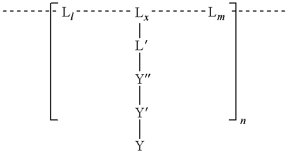
wherein, L₁ . . . Lx . . . Lm are components defining an antigenic determinant;
Lx is a component amino acid of the antigenic determinant;
L′ is a functional group of Lx;
Y″ is a molecule, a covalent bond or a linker;
Y′ an optional charged or neutral group;
Y is an electrophilic group that reacts covalently with an antibody that binds to said antigenic determinant;
n is an integer from 1 to 1000; and
m is an integer from 4 to 30.

8. The polypeptide or electrophilic analog thereof of claim 7, wherein Y″, Y′ or Y further comprises a water-binding group as a terminal or internal component.

9. The polypeptide or electrophilic analog thereof of claim 7, wherein the water-binding group binds a metal ion that chelates one or more water molecules.

10. The polypeptide or electrophilic analog thereof of claim 7, wherein the metal binding group is -(His)ₙ-, wherein n=2 or more or -Cys-X-Cys-Cys- or -Cys-X-Cys-, wherein X is an amino acid residue, ethylene diamine tetraacetic acid or diaminomethyl pyridine.

11. The polypeptide or electrophilic analog thereof of claim 7, wherein the metal is zinc, copper, nickel, cobalt, calcium, or magnesium.

12. A method for producing binary epitope specific antibodies to a B cell polypeptide antigen, comprising:
administering one or more of the polypeptide constructs of claim 1 or one or more of an electrophilic polypeptide analog thereof to a living animal.

13. The method of claim 12, further comprising:
administering one or more immunological adjuvants effective to stimulate T-cell independent or T-cell dependent B cell antibody production to the living animal.

14. The method of claim 12, wherein the polypeptide antigen is HIV gp120, Tat, Protein A, or Protein L.

15. The method of claim 12, wherein the antibodies recognize amino acid residues 421-433 and residues 301-311 of HIV gp120 epitopes.

16. The method of claim 12, wherein said binary epitope specific antibodies catalyze the hydrolysis of the native polypeptide antigen or covalently bind the native polypeptide antigen, e.g., gp120 expressed on the surface of HIV, thereby neutralizing HIV.

17. An antibody with binary specificity to a B cell polypeptide antigen produced by the method of claim 12.

18. A method for treating HIV infection in a subject, comprising:
administering an immunologically effective amount of the antibody of claim 17 to the subject.

19. A method for increasing production of antibodies recognizing the B cell superantigenic site of HIV gp120, comprising:
administering one or both of a polyclonal B cell stimulant or an electrophilic analog thereof to an living animal.

20. The method of claim 19, further comprising:
administering one or more immunological adjuvants effective to stimulate T cell-independent B cell antibody production to the living animal.

21. The method of claim 19, wherein the polyclonal B cell stimulant is one or more of pokeweed mitogen, lipopolysaccharide, phytohemagglutinin, or CpG.

22. The method of claim 19, wherein the polyclonal B cell stimulant is a superantigen that is not gp120 but can bind some or all of the antibody variable domain amino acids that bind gp120.

23. The method of claim 22, wherein the polyclonal B cell stimulant is Staphylococcal Protein A.

24. The method of claim 23, wherein the polyclonal B cell stimulant is a superantigenic domain of Protein A, an oligomer of the superantigenic domain of Protein A or Protein A labeled with iodine.

25. The method of claim 19, wherein the stimulant further comprises electrophilic groups that stimulate production of antibodies effective to catalyze the hydrolysis of gp120 or to covalently bind gp120.

26. The method of claim 19, wherein the electrophilic analog of the polyclonal B cell stimulant has one or more epitopes having the structure: 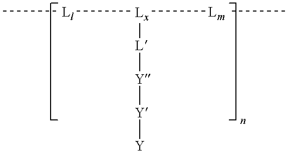
wherein L₁ . . . Lx . . . Lm are components defining an antigenic determinant;
Lx is a component amino acid of the antigenic determinant;
L′ is a functional group of Lx;
Y″ is a molecule, a covalent bond or a linker;
Y′ an optional charged or neutral group;
Y is an electrophilic group that reacts covalently with an antibody that binds to the antigenic determinant;
n is an integer from 1 to 1000; and
m is an integer from 4 to 30.

27. The method of claim 26, wherein Y″, Y′ or Y further comprises a water-binding group as a terminal or internal component.

28. The method of claim 27, wherein the water-binding group located in Y″, Y′ or Y is composed of a site that binds a metal ion which chelates one or more water molecules.

29. The method of claim 28, in which the metal binding group is selected from: -(His)ₙ- where n=2 or more, -Cys-X-Cys-Cys- or -Cys-X-Cys- wherein X is an amino acid residue, ethylene diamine tetraacetic acid or diaminomethyl pyridine.

30. An antibody recognizing the B cell superantigenic site of HIV gp120 produced by the method of claim 19.

31. A method for treating HIV infection in a subject, comprising:
administering an immunologically effective amount of the antibody of claim 30 to the subject.

32. A method for stimulating increased production of antibodies comprising:
administering to a living animal a combination of a dual epitope polypeptide and electrophilic analog thereof with an electrophilic analog of a polyclonal B cell stimulant.

33. A method for isolating an individual antibody or antibody fragment thereof having a unique sequence and binary epitope specificity from an antibody repertoire, comprising:
displaying the antibody repertoire on the surface of phage particles; and
screening the antibody repertoire with a polypeptide or a polypeptide electrophilic analog thereof or a stimulant or stimulant electrophilic analog thereof, wherein an antibody or antibody fragment thereof reacting with the polypeptide, the stimulant or the electrophilic analogs thereof thereby isolates the binary epitope specific antibody or antibody fragment thereof from the antibody repertoire.

34. The method of claim 33, wherein the polypeptide or poly peptide electrophilic analog thereof is covalently oligomerized gp120.

35. The method of claim 33, wherein the polypeptide or polypeptide electrophilic analog thereof comprises a superantigenic gp120 epitope linked to another gp120 epitope by a peptide linker.

36. The method of claim 35, wherein the superantigen epitope comprises amino acid residues 421-433 of gp120 and the other epitope comprises amino acid residues 301-311 of gp120.

37. The method of claim 35, wherein the peptide linker has a sufficient number of amino acids such that the linker length approximates the distance between the two epitopes in native gp120.

38. The method of claim 35, wherein one or both epitopes further comprise one or more electrophilic groups therewithin, said electrophilic groups effective to react with the binary specific antibodies.

39. The method of claim 33, wherein the stimulant is a superantigen that is not gp120 but can bind some or all of the antibody variable domain amino acids that bind gp120.

40. The method of claim 33, wherein stimulant is Staphylococcal Protein A.

41. The method of claim 33, wherein the stimulant is a superantigenic domain of Protein A, an oligomer of the superantigenic domain of Protein A or Protein A labeled with iodine.

42. The method of claim 33, wherein the stimulant further comprises electrophilic groups that stimulate production of antibodies effective to catalyze the hydrolysis of gp120 or to covalently bind gp120.

43. The method of claim 33, wherein the polypeptide electrophilic analog and the stimulant electrophilic analog comprise epitopes having the structure: 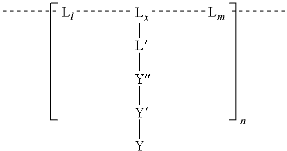
wherein, L₁ . . . Lx . . . Lm are components defining an antigenic determinant;
Lx is a component amino acid of the antigenic determinant;
L′ is a functional group of Lx;
Y″ is a molecule, a covalent bond or a linker;
Y′ an optional charged or neutral group;
Y is an electrophilic group that reacts covalently with an antibody that binds to said antigenic determinant;
n is an integer from 1 to 1000; and
m is an integer from 4 to 30.

44. The method of claim 33, wherein the antibody repertoire comprises B cells from a living organism that produce the binary epitope specific antibodies or genes encoding the antibodies isolated from the B cells.

45. The method of claim 33, wherein the antibody or antibody fragment thereof reversibly binds an antigen, covalently binds an antigen or catalyzes the hydrolysis of the antigen.

46. The method of claim 33, wherein the antibodies recognize the antigen HIV gp120.

47. An electrophilic analog of a lipid, a polysaccharide or a lipopolysaccharide having the structure: 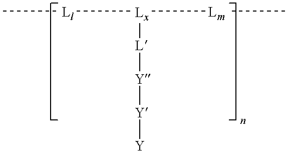
wherein, L₁ . . . Lx . . . Lm are components defining a receptor binding determinant;
Lx is a component sugar or lipid of the receptor binding determinant;
L′ is a functional group of Lx;
Y″ is a molecule, a covalent bond or a linker;
Y′ an optional charged or neutral group;
Y is an electrophilic group that reacts covalently with a cellular receptor that binds to said receptor binding determinant, or an acyl group; wherein, optionally, Y″, Y′ or Y comprises a water-binding group as a terminal or internal component;
n is an integer from 1 to 1000; and
m is from 1 to 1000.

48. An electrophilic analog of an oligonucleotide having the structure: 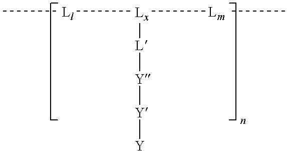
wherein, L₁ . . . Lx . . . Lm are nucleotide components defining a receptor binding determinant;
Lx is a component nucleotide of the receptor binding determinant;
L′ is a functional group of Lx;
Y″ is a molecule, a covalent bond or a linker;
Y′ an optional charged or neutral group;
Y is an electrophilic group that reacts covalently with a cellular receptor that binds to said receptor binding determinant, wherein, optionally, Y″, Y′ or Y contains a water-binding group as a terminal or internal component;
n is an integer from 1 to 1000; and
m is from 1 to 1000.


SEQUENCE LISTING (45 sequences)

SEQ ID NO: 1 - protein, 4 aa, artificial sequence
  metal binding peptide; Xaa at position 2 is any amino acid
CXCC

SEQ ID NO: 2 - protein, 3 aa, artificial sequence
  metal binding peptide; Xaa at position 2 is any amino acid
CXC

SEQ ID NO: 3 - protein, 19 aa, artificial sequence
  HIV gp120 peptide 293-311
LNESVQINCTRPNYNKRKR

SEQ ID NO: 4 - protein, 15 aa, artificial sequence
  HIV gp120 peptide 297-311
VQINCTRPNYNKRKR

SEQ ID NO: 5 - protein, 15 aa, artificial sequence
  HIV gp120 peptide 421-435
KQIINMWQEVGKAMY

SEQ ID NO: 6 - protein, 15 aa, artificial sequence
  HIV gp120 peptide 465-479
NDTEIFRPGGGDMRD

SEQ ID NO: 7 - protein, 15 aa, artificial sequence
  HIV gp120 peptide 145-159
NNNSNSEGTIKGGEM

SEQ ID NO: 8 - protein, 15 aa, artificial sequence
  HIV gp120 peptide 217-231
PIPIHYCAPAGFAIL

SEQ ID NO: 9 - protein, 15 aa, artificial sequence
  HIV gp120 peptide 301-315
CTRPNYNKRKRIHIG

SEQ ID NO: 10 - protein, 11 aa, artificial sequence
  HIV gp120 probe epitope sequence E-421-431
KQIINMWQEVG

SEQ ID NO: 11 - protein, 125 aa, artificial sequence
  HIV gp120 monoclonal antibody 18/2 variable heavy chain peptide sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGQVLYYGSGSYHWFDPWGQGTLVTVSS

SEQ ID NO: 12 - protein, 123 aa, artificial sequence
  HIV gp120 monoclonal antibody YZ18 variable heavy chain peptide sequence
EVQLQQSGTVLARPGASVKMSCKASGYTFTSYWMHWVKQRPGQGLEWIGAIYPGNSDTSYNQKFKGKAKLTAVTSASTAYMELSSLTNEDSAVYYCTRWPHYYGGSRYYFDYWGQGTTLTVSS

SEQ ID NO: 13 - protein, 118 aa, artificial sequence
  HIV gp120 monoclonal antibody YZ23 variable heavy chain peptide sequence
EVKLQQSGAELVRPGASVKMSCKASGYRFTSYNMHWVKQTPRQGLEWIGAIYPGNGDTSYNQKFKGKATLTVDKSSSTAYMQLSSLTSEDSAVYFCARGRLSLGFDYWGQGSTLTVSS

SEQ ID NO: 14 - protein, 12 aa, artificial sequence
  HIV gp120 probe epitope sequence E-300-311
NCTRPNYNKRKR

SEQ ID NO: 15 - protein, 15 aa, artificial sequence
  tetanus toxoid peptide
QYIKANSKFIGITEL

SEQ ID NO: 16 - protein, 16 aa, artificial sequence
  HIV gp120 epitope sequence E-421-436
KQIINMWQEVGKAMYA

SEQ ID NO: 17 - protein, 11 aa, artificial sequence
  HIV gp120 epitope sequence E-301-311
CTRPNYNKRKR

SEQ ID NO: 18 - protein, 11 aa, artificial sequence
  HIV gp120 constrained epitope sequence E-421- 431 where Xaa at position 6 is ornithine
KQIINXWQEVD

SEQ ID NO: 19 - protein, 5 aa, artificial sequence
  N-terminus of HIV gp120
IPGRK

SEQ ID NO: 20 - protein, 5 aa, artificial sequence
  HIV gp120 peptide 322-326
TTKNI

SEQ ID NO: 21 - protein, 5 aa, artificial sequence
  HIV gp120 peptide 84-88
ELVNV

SEQ ID NO: 22 - protein, 5 aa, artificial sequence
  HIV gp120 peptide 433-437
AMYAP

SEQ ID NO: 23 - protein, 11 aa, artificial sequence
  scrambled peptide from HIV gp120 421-433
IWNQVQEIKGM

SEQ ID NO: 24 - protein, 11 aa, Human Immunodeficiency Virus
  HIV-1 gp120 peptide 301-311 consensus sequence
CTRPNNNTRKS

SEQ ID NO: 25 - protein, 13 aa, Human Immunodeficiency Virus
  HIV gp120 peptide 421-433
KQIINMWQEVGKA

SEQ ID NO: 26 - protein, 13 aa, Human Immunodeficiency Virus
  HIV gp120 peptide 421-433 consensus sequence
KQIINMWQRVGKA

SEQ ID NO: 27 - protein, 13 aa, Human Immunodeficiency Virus
  HIV gp120 peptide 421-433 consensus sequence
KQIINMWQGVGKA

SEQ ID NO: 28 - protein, 13 aa, Human Immunodeficiency Virus
  HIV gp120 peptide 421-433 consensus sequence
KQIINMWQEVGQA

SEQ ID NO: 29 - protein, 13 aa, Human Immunodeficiency Virus
  HIV gp120 peptide 421-433 consensus sequence
KQIINMWQEVGRA

SEQ ID NO: 30 - protein, 13 aa, Human Immunodeficiency Virus
  HIV gp120 peptide 421-433 consensus sequence
KQIINMWQRVGQA

SEQ ID NO: 31 - protein, 13 aa, Human Immunodeficiency Virus
  HIV gp120 peptide 421-433 consensus sequence
KQIINMWQRVGRA

SEQ ID NO: 32 - protein, 13 aa, Human Immunodeficiency Virus
  HIV gp120 peptide 421-433 consensus sequence
KQIINMWGRVGQA

SEQ ID NO: 33 - protein, 13 aa, Human Immunodeficiency Virus
  HIV gp120 peptide 421-433 consensus sequence
KQIINMWQGVGRA

SEQ ID NO: 34 - protein, 13 aa, Human Immunodeficiency Virus
  HIV gp120 peptide 421-433 consensus sequence
KQIVNMWQEVGKA

SEQ ID NO: 35 - protein, 13 aa, Human Immunodeficiency Virus
  HIV gp120 peptide 421-433 consensus sequence
KQIVNMWQRVGKA

SEQ ID NO: 36 - protein, 13 aa, Human Immunodeficiency Virus
  HIV gp120 peptide 421-433 consensus sequence
KQIVNMWQGVGKA

SEQ ID NO: 37 - protein, 13 aa, Human Immunodeficiency Virus
  HIV gp120 peptide 421-433 consensus sequence
KQIVNMWQEVGQA

SEQ ID NO: 38 - protein, 13 aa, Human Immunodeficiency Virus
  HIV gp120 peptide 421-433 consensus sequence
KQIVNMWQEVGRA

SEQ ID NO: 39 - protein, 13 aa, Human Immunodeficiency Virus
  HIV gp120 peptide 421-433 consensus sequence
KQIVNMWQRVGQA

SEQ ID NO: 40 - protein, 13 aa, Human Immunodeficiency Virus
  HIV gp120 peptide 421-433 consensus sequence
KQIVNMWQRVGRA

SEQ ID NO: 41 - protein, 13 aa, Human Immunodeficiency Virus
  HIV gp120 peptide 421-433 consensus sequence
KQIVNMWQGVGQA

SEQ ID NO: 42 - protein, 13 aa, Human Immunodeficiency Virus
  HIV gp120 peptide 421-433 consensus sequence
KQIVNMWQGVGRA

SEQ ID NO: 43 - protein, 19 aa, artificial sequence
  HIV gp120 peptide 293-311 where Xaa at position 9 is S-acetamidomethyl-Cys
LNESVQINXTRPNYNKRKR

SEQ ID NO: 44 - protein, 12 aa, artificial sequence
  peptide from calcium-binding loop of calmodulin
DKDGDGYISAAE

SEQ ID NO: 45 - DNA, 24 bp, artificial sequence
  electrophilic E-CpG sequence derived from the TLR9 agonist, ODN2006
tcgtcgttttgtcgttttgtcgtt